SEQUENCE LISTING (307 sequences)

SEQ ID NO: 1 - protein, 15 aa, Artificial Sequence
  synthetic sequence
GGGSEGGGSEGGGSE

SEQ ID NO: 2 - protein, 24 aa, Artificial Sequence
  synthetic sequence
MKWVTFISLLFLFSSAYSRGVFRR

SEQ ID NO: 3 - protein, 585 aa, Artificial Sequence [sequence omitted]

SEQ ID NO: 4 - protein, 5 aa, Artificial Sequence
  synthetic sequence
GGGGS

SEQ ID NO: 5 - protein, 10 aa, Artificial Sequence
  synthetic sequence
GGGGSGGGGS

SEQ ID NO: 6 - protein, 15 aa, Artificial Sequence
  synthetic sequence
GGGGSGGGGSGGGGS

SEQ ID NO: 7 - protein, 20 aa, Artificial Sequence
  synthetic sequence
GGGGSGGGGSGGGGSGGGGS

SEQ ID NO: 8 - protein, 5 aa, Artificial Sequence
  synthetic sequence
GGGGA

SEQ ID NO: 9 - protein, 10 aa, Artificial Sequence
  synthetic sequence
GGGGAGGGGA

SEQ ID NO: 10 - protein, 15 aa, Artificial Sequence
  synthetic sequence
GGGGAGGGGAGGGGA

SEQ ID NO: 11 - protein, 20 aa, Artificial Sequence
  synthetic sequence
GGGGAGGGGAGGGGAGGGGA

SEQ ID NO: 12 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 118 aa, Artificial Sequence
  synthetic sequence
QVQLVQSGAEVKKPGASVKVSCKASGGSFTGYYMHWVRQAPGQGLEWMGWINPNDGAIHYAQNFQGRVTMTRDTSTSTVYMELSSLRSEDTAVYYCARDTVTGDFDYWGQGTLVTVSS

SEQ ID NO: 14 - protein, 107 aa, Artificial Sequence
  synthetic sequence
DIQMTQSPSSLSASVGDRVTITCQASHDIDKYLNWYQQKPGKAPKLLIYAASSLQSGVPSRFSGSGSGTDFTLTISSLQPEDFATYYCQQANSLPLTFGGGTKVEIK

SEQ ID NO: 15 - protein, 9 aa, Artificial Sequence
  synthetic sequence
GSFTGYYMH

SEQ ID NO: 16 - protein, 13 aa, Artificial Sequence
  synthetic sequence
GWINPNDGAIHYA

SEQ ID NO: 17 - protein, 13 aa, Artificial Sequence
  synthetic sequence
CARDTVTGDFDYW

SEQ ID NO: 18 - protein, 11 aa, Artificial Sequence
  synthetic sequence
QASHDIDKYLN

SEQ ID NO: 19 - protein, 5 aa, Artificial Sequence
  synthetic sequence
SSLQS

SEQ ID NO: 20 - protein, 9 aa, Artificial Sequence
  synthetic sequence
QQANSLPLT

SEQ ID NO: 21 - protein, 121 aa, Artificial Sequence
  synthetic sequence
QVQLVQSGAEVKKPGASVKVSCKASGGTFSRYAVSWVRQAPGQGLEWMGWINPNSGGTSYAQRFQGRVTMTRDTSTSTVYMELSSLRSEDTAVYYCAKQGDYGGGYYFDYWGQGTLVTVSS

SEQ ID NO: 22 - protein, 107 aa, Artificial Sequence
  synthetic sequence
DIQMTQSPSSLSASVGDRVTITCRASQSISSWLAWYQQKPGKAPKLLIYKTSTLESGVPSRFSGSGSGTDFTLTISSLQPEDFATYYCQQSYSTPFTFGQGTKVEIK

SEQ ID NO: 23 - protein, 9 aa, Artificial Sequence
  synthetic sequence
GTFSRYAVS

SEQ ID NO: 24 - protein, 13 aa, Artificial Sequence
  synthetic sequence
GWINPNSGGTSYA

SEQ ID NO: 25 - protein, 16 aa, Artificial Sequence
  synthetic sequence
CAKQGDYGGGYYFDYW

SEQ ID NO: 26 - protein, 11 aa, Artificial Sequence
  synthetic sequence
RASQSISSWLA

SEQ ID NO: 27 - protein, 5 aa, Artificial Sequence
  synthetic sequence
STLES

SEQ ID NO: 28 - protein, 9 aa, Artificial Sequence
  synthetic sequence
QQSYSTPFT

SEQ ID NO: 29 - protein, 120 aa, Artificial Sequence
  synthetic sequence
QVQLVQSGAEVKKPGASVKVSCKASGGTFSSYAISWVRQAPGQGLEWMGWMNPNSGNTGYAQKFQGRVTMTRDTSTSTVYMELSSLRSEDTAVYYCARVGYSYGYGMDVWGQGTTVTVSS

SEQ ID NO: 30 - protein, 107 aa, Artificial Sequence
  synthetic sequence
DIQMTQSPSSLSASVGDRVTITCRASQSINNWLAWYQQKPGKAPKLLIYAASSLQSGVPSRFSGSGSGTDFTLTISSLQPEDFATYYCQQSYSTPFTFGPGTKVDIK

SEQ ID NO: 31 - protein, 9 aa, Artificial Sequence
  synthetic sequence
GTFSSYAIS

SEQ ID NO: 32 - protein, 13 aa, Artificial Sequence
  synthetic sequence
GWMNPNSGNTGYA

SEQ ID NO: 33 - protein, 15 aa, Artificial Sequence
  synthetic sequence
CARVGYSYGYGMDVW

SEQ ID NO: 34 - protein, 11 aa, Artificial Sequence
  synthetic sequence
RASQSINNWLA

SEQ ID NO: 35 - protein, 113 aa, Artificial Sequence
  synthetic sequence
QVQLVQSGAEVKKPGASVKVSCKASGYSFTTYYMHWVRQAPGQGLEWMGIINPSGGSTSYAQKFQGRVTMTRDTSTSTVYMELSSLRSEDTAVYYCASGWVYWGQGTLVTVSS

SEQ ID NO: 36 - protein, 107 aa, Artificial Sequence
  synthetic sequence
DIQMTQSPSSLSASVGDRVTITCQASRDIKNYLAWYQQKPGKAPKLLIYAASSLQSGVPSRFSGSGSGTDFTLTISSLQPEDFATYYCQQSYSTPPTFGPGTKVDIK

SEQ ID NO: 37 - protein, 9 aa, Artificial Sequence
  synthetic sequence
YSFTTYYMH

SEQ ID NO: 38 - protein, 13 aa, Artificial Sequence
  synthetic sequence
GIINPSGGSTSYA

SEQ ID NO: 39 - protein, 8 aa, Artificial Sequence
  synthetic sequence
CASGWVYW

SEQ ID NO: 40 - protein, 11 aa, Artificial Sequence
  synthetic sequence
QASRDIKNYLA

SEQ ID NO: 41 - protein, 9 aa, Artificial Sequence
  synthetic sequence
QQSYSTPPT

SEQ ID NO: 42 - protein, 119 aa, Artificial Sequence
  synthetic sequence
EVQLLESGGGLVQPGGSLRLSCAASGFTFSSYAMSWVRQAPGKGLEWVAAIWSDGSHQYYADSVKGRFTISRDNSKNTLYLQMNSLRAEDTAVYYCARGLGVERGLDYWGQGTLVTVSS

SEQ ID NO: 43 - protein, 107 aa, Artificial Sequence
  synthetic sequence
DIQMTQSPSSLSASVGDRVTITCRASQSISSWLAWYQQKPGKAPKLLIYAASTLQSGVPSRFSGSGSGTDFTLTISSLQPEDFATYYCQQSYSTPLTFGQGTKVEIK

SEQ ID NO: 44 - protein, 9 aa, Artificial Sequence
  synthetic sequence
FTFSSYAMS

SEQ ID NO: 45 - protein, 13 aa, Artificial Sequence
  synthetic sequence
AAIWSDGSHQYYA

SEQ ID NO: 46 - protein, 14 aa, Artificial Sequence
  synthetic sequence
CARGLGVERGLDYW

SEQ ID NO: 47 - protein, 5 aa, Artificial Sequence
  synthetic sequence
STLQS

SEQ ID NO: 48 - protein, 9 aa, Artificial Sequence
  synthetic sequence
QQSYSTPLT

SEQ ID NO: 49 - protein, 124 aa, Artificial Sequence
  synthetic sequence
EVQLLESGGGLVQPGGSLRLSCAASGFTFSNYPMHWVRQAPGKGLEWVALISDDGTNEHYADSVKGRFTISRDNSKNTLYLQMNSLRAEDTAVYYCARDSKFANYYYYYDMDVWGQGTTVTVSS

SEQ ID NO: 50 - protein, 107 aa, Artificial Sequence
  synthetic sequence
DIQMTQSPSSLSASVGDRVTITCRASQSINNYLSWYQQKPGKAPKLLIYDASNLETGVPSRFSGSGSGTDFTLTISSLQPEDFATYYCQQSYSTPLTFGPGTKVDIK

SEQ ID NO: 51 - protein, 9 aa, Artificial Sequence
  synthetic sequence
FTFSNYPMH

SEQ ID NO: 52 - protein, 13 aa, Artificial Sequence
  synthetic sequence
ALISDDGTNEHYA

SEQ ID NO: 53 - protein, 19 aa, Artificial Sequence
  synthetic sequence
CARDSKFANYYYYYDMDVW

SEQ ID NO: 54 - protein, 11 aa, Artificial Sequence
  synthetic sequence
RASQSINNYLS

SEQ ID NO: 55 - protein, 5 aa, Artificial Sequence
  synthetic sequence
SNLET

SEQ ID NO: 56 - protein, 124 aa, Artificial Sequence
  synthetic sequence
QVQLVQSGAEVKKPGASVKVSCKASGYSFTGHYIHWVRQAPGQGLEWMGIINPNGGSTTYAQKLQGRVTMTRDTSTSTVYMELSSLRSEDTAVYYCARGKFDFYGDYVTAFDIWGQGTMVTVSS

SEQ ID NO: 57 - protein, 113 aa, Artificial Sequence
  synthetic sequence
DIVMTQSPDSLAVSLGERATINCRSSQSILYSSNNRDYLAWYQQKPGQPPKLLIYWASTRQSGVPDRFSGSGSGTDFTLTISSLQAEDVAVYYCQQYYSIPVTFGGGTKVEIK

SEQ ID NO: 58 - protein, 9 aa, Artificial Sequence
  synthetic sequence
YSFTGHYIH

SEQ ID NO: 59 - protein, 13 aa, Artificial Sequence
  synthetic sequence
GIINPNGGSTTYA

SEQ ID NO: 60 - protein, 19 aa, Artificial Sequence
  synthetic sequence
CARGKFDFYGDYVTAFDIW

SEQ ID NO: 61 - protein, 17 aa, Artificial Sequence
  synthetic sequence
RSSQSILYSSNNRDYLA

SEQ ID NO: 62 - protein, 5 aa, Artificial Sequence
  synthetic sequence
STRQS

SEQ ID NO: 63 - protein, 9 aa, Artificial Sequence
  synthetic sequence
QQYYSIPVT

SEQ ID NO: 64 - protein, 128 aa, Artificial Sequence
  synthetic sequence
QVQLVQSGAEVKKPGASVKVSCKASGYTFSNYDMNWVRQAPGQGLEWMGWMNPNSGHTGSAPKFQGRVTMTRDTSTSTVYMELSSLRSEDTAVYYCARGAFGLHLGELSLHYYGMDVWGQGTTVTVSS

SEQ ID NO: 65 - protein, 107 aa, Artificial Sequence
  synthetic sequence
DIQMTQSPSSLSASVGDRVTITCRASQSINNWLAWYQQKPGKAPKLLIYAASSLQGGVPSRFSGSGSGTDFTLTISSLQPEDFATYYCQQSYSFPYTFGQGTKLEIK

SEQ ID NO: 66 - protein, 9 aa, Artificial Sequence
  synthetic sequence
YTFSNYDMN

SEQ ID NO: 67 - protein, 13 aa, Artificial Sequence
  synthetic sequence
GWMNPNSGHTGSA

SEQ ID NO: 68 - protein, 23 aa, Artificial Sequence
  synthetic sequence
CARGAFGLHLGELSLHYYGMDVW

SEQ ID NO: 69 - protein, 5 aa, Artificial Sequence
  synthetic sequence
SSLQG

SEQ ID NO: 70 - protein, 9 aa, Artificial Sequence
  synthetic sequence
QQSYSFPYT

SEQ ID NO: 71 - protein, 117 aa, Artificial Sequence
  synthetic sequence
QVQLVQSGAEVKKPGSSVKVSCKASGYTFTGYYMHWVRQAPGQGLEWMGKIVPMFDAANYAPKFQGRVTITADESTSTAYMELSSLRSEDTAVYYCARGPKWELDTWGQGTLVTVSS

SEQ ID NO: 72 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTLTISSLQPEDFATYYCQQANSFPVTFGGGTKVEIK

SEQ ID NO: 73 - protein, 9 aa, Artificial Sequence
  synthetic sequence
YTFTGYYMH

SEQ ID NO: 74 - protein, 13 aa, Artificial Sequence
  synthetic sequence
GKIVPMFDAANYA

SEQ ID NO: 75 - protein, 12 aa, Artificial Sequence
  synthetic sequence
CARGPKWELDTW

SEQ ID NO: 76 - protein, 11 aa, Artificial Sequence
  synthetic sequence
RASQSISRWLA

SEQ ID NO: 77 - protein, 9 aa, Artificial Sequence
  synthetic sequence
QQANSFPVT

SEQ ID NO: 78 - protein, 124 aa, Artificial Sequence
  synthetic sequence
QVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCAKTAGYDWLPSGLGMDVWGQGTTVTVSS

SEQ ID NO: 79 - protein, 107 aa, Artificial Sequence
  synthetic sequence
DIQMTQSPSSLSASVGDRVTITCRASQSINSWLAWYQQKPGKAPKLLIYYASSLQSGVPSRFSGSGSGTDFTLTISSLQPEDFATYYCQQGYSVPLSFGQGTKLEIK

SEQ ID NO: 80 - protein, 19 aa, Artificial Sequence
  synthetic sequence
CAKTAGYDWLPSGLGMDVW

SEQ ID NO: 81 - protein, 11 aa, Artificial Sequence
  synthetic sequence
RASQSINSWLA

SEQ ID NO: 82 - protein, 9 aa, Artificial Sequence
  synthetic sequence
QQGYSVPLS

SEQ ID NO: 83 - protein, 118 aa, Artificial Sequence
  synthetic sequence
QVQLVQSGAEVKKPGSSVKVSCKASGYTFSNYGITWVRQAPGQGLEWMGGIIPIFGSTASYAQKFQGRVTITADESTSTAYMELSSLRSEDTAVYYCARWRSDAFDIWGQGTMVTVSS

SEQ ID NO: 84 - protein, 107 aa, Artificial Sequence
  synthetic sequence
DIQMTQSPSSLSASVGDRVTITCRASQGISNWLAWYQQKPGKAPKLLIYDASNLETGVPSRFSGSGSGTDFTLTISSLQPEDFATYYCQQSYSTPLTFGGGTKVEIK

SEQ ID NO: 85 - protein, 9 aa, Artificial Sequence
  synthetic sequence
YTFSNYGIT

SEQ ID NO: 86 - protein, 14 aa, Artificial Sequence
  synthetic sequence
GGIIPIFGSTASYA

SEQ ID NO: 87 - protein, 12 aa, Artificial Sequence
  synthetic sequence
CARWRSDAFDIW

SEQ ID NO: 88 - protein, 11 aa, Artificial Sequence
  synthetic sequence
RASQGISNWLA

SEQ ID NO: 89 - protein, 120 aa, Artificial Sequence
  synthetic sequence
QVQLVQSGAEVKKPGASVKVSCKASGGTFSTYAISWVRQAPGQGLEWMGWINPNSGGTNYAQKFQGRVTMTRDTSTSIVYMELSSLRSEDTAVYYCARVNYDFYYGMDVWGQGTTVTVSS

SEQ ID NO: 90 - protein, 107 aa, Artificial Sequence
  synthetic sequence
DIQMTQSPSSLSASVGDRVTITCRASQGIRNDLGWYQQKPGKAPKLLIYRASTLNSGVPSRFSGSGSGTDFTLTISSLQPEDFATYYCQQSYSTPFTFGPGTKVDIK

SEQ ID NO: 91 - protein, 9 aa, Artificial Sequence
  synthetic sequence
GTFSTYAIS

SEQ ID NO: 92 - protein, 13 aa, Artificial Sequence
  synthetic sequence
GWINPNSGGTNYA

SEQ ID NO: 93 - protein, 15 aa, Artificial Sequence
  synthetic sequence
CARVNYDFYYGMDVW

SEQ ID NO: 94 - protein, 11 aa, Artificial Sequence
  synthetic sequence
RASQGIRNDLG

SEQ ID NO: 95 - protein, 5 aa, Artificial Sequence
  synthetic sequence
STLNS

SEQ ID NO: 96 - protein, 120 aa, Artificial Sequence
  synthetic sequence
QVQLVQSGAEVKKPGASVKVSCKASGGTFSTYAISWVRQAPGQGLEWMGWINPNSGGTNYAQKFQGRVTMTRDTSTSTVYMELSSLRSEDTAVYYCARVNYDFYYGMDVWGQGTTVTVSS

SEQ ID NO: 97 - protein, 118 aa, Artificial Sequence
  synthetic sequence
EVQLLESGGGLVQPGGSLRLSCAASGFSFSSYDMSWVRQAPGKGLEWVSGISGSGSSTYYADSVKGRFTISRDNSKNTLYLQMNSLRAEDTAVYYCASPYGMGYMDVWGKGTTVTVSS

SEQ ID NO: 98 - protein, 107 aa, Artificial Sequence
  synthetic sequence
DIQMTQSPSSLSASVGDRVTITCRASQDIANYLAWYQQKPGKAPKLLIYGASSVQTGVPSRFSGSGSGTDFTLTISSLQPEDFATYYCQQSYTTPYTFGQGTRLEIK

SEQ ID NO: 99 - protein, 9 aa, Artificial Sequence
  synthetic sequence
FSFSSYDMS

SEQ ID NO: 100 - protein, 13 aa, Artificial Sequence
  synthetic sequence
SGISGSGSSTYYA

SEQ ID NO: 101 - protein, 13 aa, Artificial Sequence
  synthetic sequence
CASPYGMGYMDVW

SEQ ID NO: 102 - protein, 11 aa, Artificial Sequence
  synthetic sequence
RASQDIANYLA

SEQ ID NO: 103 - protein, 5 aa, Artificial Sequence
  synthetic sequence
SSVQT

SEQ ID NO: 104 - protein, 9 aa, Artificial Sequence
  synthetic sequence
QQSYTTPYT

SEQ ID NO: 105 - protein, 120 aa, Artificial Sequence
  synthetic sequence
QVQLVQSGAEVKKPGASVKVSCKASGGSFNNYAISWVRQAPGQGLEWMGWINPNTGGTSYAQKFQGRVTMTRDTSTSTVYMELSSLRSEDTAVYYCARVSYGVGYYMDVWGKGTTVTVSS

SEQ ID NO: 106 - protein, 107 aa, Artificial Sequence
  synthetic sequence
DIQMTQSPSSLSASVGDRVTITCQASQDISRYLNWYQQKPGKAPKLLIYAASNLQSGVPSRFSGSGSGTDFTLTISSLQPEDFATYYCQQSYSTPFTFGPGTKVDIK

SEQ ID NO: 107 - protein, 9 aa, Artificial Sequence
  synthetic sequence
GSFNNYAIS

SEQ ID NO: 108 - protein, 13 aa, Artificial Sequence
  synthetic sequence
GWINPNTGGTSYA

SEQ ID NO: 109 - protein, 15 aa, Artificial Sequence
  synthetic sequence
CARVSYGVGYYMDVW

SEQ ID NO: 110 - protein, 11 aa, Artificial Sequence
  synthetic sequence
QASQDISRYLN

SEQ ID NO: 111 - protein, 5 aa, Artificial Sequence
  synthetic sequence
SNLQS

SEQ ID NO: 112 - protein, 107 aa, Artificial Sequence
  synthetic sequence
DIQMTQSPSSLSASVGDRVTITCQASQDISRYLNWYQQKPGKAPKLLIYAASNLQSGVPSRFSGSGSGTDSTLTISSLQPEDFATYYCQQSYSTPFTFGPGTKVDIK

SEQ ID NO: 113 - protein, 129 aa, Artificial Sequence
  synthetic sequence
QVQLVQSGAEVKKPGASVKVSCKASGYTFTDDYIHWVRQAPGQGLEWMGWMNTNSGNTGYAQKFQGRVTMTRDTSTSTVYMELSSLRSEDTAVYYCARGGSYSSGWYGRLDYYYGMDVWGQGTTVTVSS

SEQ ID NO: 114 - protein, 107 aa, Artificial Sequence
  synthetic sequence
DIQMTQSPSSLSASVGDRVTITCRASQGVGNALGWYQQKPGKAPKLLIYAASSLQSGVPSRFSGSGSGTDFTLTISSLQPEDFATYYCQQAYSFPWTFGQGTKLEIK

SEQ ID NO: 115 - protein, 9 aa, Artificial Sequence
  synthetic sequence
YTFTDDYIH

SEQ ID NO: 116 - protein, 13 aa, Artificial Sequence
  synthetic sequence
GWMNTNSGNTGYA

SEQ ID NO: 117 - protein, 24 aa, Artificial Sequence
  synthetic sequence
CARGGSYSSGWYGRLDYYYGMDVW

SEQ ID NO: 118 - protein, 11 aa, Artificial Sequence
  synthetic sequence
RASQGVGNALG

SEQ ID NO: 119 - protein, 9 aa, Artificial Sequence
  synthetic sequence
QQAYSFPWT

SEQ ID NO: 120 - protein, 113 aa, Artificial Sequence
  synthetic sequence
QVQLVQSGAEVKKPGASVKVSCKASGYTFTDYAMHWVRQAPGQGLEWMGWLNPNSGNTGYAPKFQGRVTMTRDTSTSTVYMELSSLRSEDTAVYYCAAGLFIWGQGTMVTVSS

SEQ ID NO: 121 - protein, 107 aa, Artificial Sequence
  synthetic sequence
DIQMTQSPSSLSASVGDRVTITCRASQSINRWLAWYQQKPGKAPKLLIYDASSLESGVPSRFSGSGSGTDFTLTISSLQPEDFATYYCQQSYSIPITFGQGTRLEIK

SEQ ID NO: 122 - protein, 9 aa, Artificial Sequence
  synthetic sequence
YTFTDYAMH

SEQ ID NO: 123 - protein, 13 aa, Artificial Sequence
  synthetic sequence
GWLNPNSGNTGYA

SEQ ID NO: 124 - protein, 8 aa, Artificial Sequence
  synthetic sequence
CAAGLFIW

SEQ ID NO: 125 - protein, 11 aa, Artificial Sequence
  synthetic sequence
RASQSINRWLA

SEQ ID NO: 126 - protein, 5 aa, Artificial Sequence
  synthetic sequence
SSLES

SEQ ID NO: 127 - protein, 9 aa, Artificial Sequence
  synthetic sequence
QQSYSIPIT

SEQ ID NO: 128 - protein, 120 aa, Artificial Sequence
  synthetic sequence
QVQLVQSGAEVKKPGSSVKVSCKASGGTFSSYAISWVRQAPGQGLEWMGGIIPGFGSPNYAPNFQGRVTITADESTSTAYMELSSLRSEDTAVYYCTTEYCSSTSCSDYWGQGTLVTVSS

SEQ ID NO: 129 - protein, 112 aa, Artificial Sequence
  synthetic sequence
DIVMTQSPLSLPVTPGEPASISCRSSQSLLHSNGYNYLDWYLQKPGQSPQLLIYQGSNRAPGVPDRFSGSGSGTDFTLKISRVEAEDVGVYYCMQALQTPLTFGQGTKVEIK

SEQ ID NO: 130 - protein, 13 aa, Artificial Sequence
  synthetic sequence
GGIIPGFGSPNYA

SEQ ID NO: 131 - protein, 15 aa, Artificial Sequence
  synthetic sequence
CTTEYCSSTSCSDYW

SEQ ID NO: 132 - protein, 16 aa, Artificial Sequence
  synthetic sequence
RSSQSLLHSNGYNYLD

SEQ ID NO: 133 - protein, 5 aa, Artificial Sequence
  synthetic sequence
SNRAP

SEQ ID NO: 134 - protein, 9 aa, Artificial Sequence
  synthetic sequence
MQALQTPLT

SEQ ID NO: 135 - protein, 125 aa, Artificial Sequence
  synthetic sequence
QVQLVQSGAEVKKPGASVKVSCKASGYTFSDHYMHWVRQAPGQGLEWMGTINPSGGRTSYAQKFQGRVTMTRDTSTSTVYMELSSLRSEDTAVYYCAADNGHASGWLYYYGMDVWGQGTTVTVSS

SEQ ID NO: 136 - protein, 107 aa, Artificial Sequence
  synthetic sequence
DIQMTQSPSSLSASVGDRVTITCRASQSISNWVAWYQQKPGKAPKLLIYRASTLQSGVPSRFSGSGSGTDFTLTISSLQPEDFATYYCQQSHSLPLTFGPGTKVDIK

SEQ ID NO: 137 - protein, 9 aa, Artificial Sequence
  synthetic sequence
YTFSDHYMH

SEQ ID NO: 138 - protein, 13 aa, Artificial Sequence
  synthetic sequence
GTINPSGGRTSYA

SEQ ID NO: 139 - protein, 20 aa, Artificial Sequence
  synthetic sequence
CAADNGHASGWLYYYGMDVW

SEQ ID NO: 140 - protein, 11 aa, Artificial Sequence
  synthetic sequence
RASQSISNWVA

SEQ ID NO: 141 - protein, 9 aa, Artificial Sequence
  synthetic sequence
QQSHSLPLT

SEQ ID NO: 142 - protein, 116 aa, Artificial Sequence
  synthetic sequence
EVQLLESGGGLVQPGGSLRLSCAASGFTFSSYAMSWVRQAPGKGLEWVSGISGGGGTTYYADSVKGRFTISRDNSKNTLYLQMNSLRAEDTAVYYCASEYYGMDVWGQGTTVTVSS

SEQ ID NO: 143 - protein, 107 aa, Artificial Sequence
  synthetic sequence
DIQMTQSPSSLSASVGDRVTITCRASQSISGWLAWYQQKPGKAPKLLIYAASSLQSGVPSRFSGSGSGTDFTLTISSLQPEDFATYYCQQYRNFPYTFGQGTKLEIK

SEQ ID NO: 144 - protein, 13 aa, Artificial Sequence
  synthetic sequence
SGISGGGGTTYYA

SEQ ID NO: 145 - protein, 11 aa, Artificial Sequence
  synthetic sequence
CASEYYGMDVW

SEQ ID NO: 146 - protein, 11 aa, Artificial Sequence
  synthetic sequence
RASQSISGWLA

SEQ ID NO: 147 - protein, 9 aa, Artificial Sequence
  synthetic sequence
QQYRNFPYT

SEQ ID NO: 148 - protein, 113 aa, Artificial Sequence
  synthetic sequence
QVQLVQSGAEVKKPGASVKVSCKASGYTFSGYYMHWVRQAPGQGLEWMGVINPSGGSTSYAQKFQGRVTMTRDTSTSTVYMELSSLRSEDTAVYYCAEGFDYWGQGTLVTVSS

SEQ ID NO: 149 - protein, 107 aa, Artificial Sequence
  synthetic sequence
EIVMTQSPATLSVSPGERATLSCRASQGVGRSLAWYQQKPGQAPRLLIYGASTRATGIPARFSGSGSGTEFTLTISSLQSEDFAVYYCQQYYTTPITFGQGTRLEIK

SEQ ID NO: 150 - protein, 9 aa, Artificial Sequence
  synthetic sequence
YTFSGYYMH

SEQ ID NO: 151 - protein, 13 aa, Artificial Sequence
  synthetic sequence
GVINPSGGSTSYA

SEQ ID NO: 152 - protein, 8 aa, Artificial Sequence
  synthetic sequence
CAEGFDYW

SEQ ID NO: 153 - protein, 11 aa, Artificial Sequence
  synthetic sequence
RASQGVGRSLA

SEQ ID NO: 154 - protein, 5 aa, Artificial Sequence
  synthetic sequence
STRAT

SEQ ID NO: 155 - protein, 9 aa, Artificial Sequence
  synthetic sequence
QQYYTTPIT

SEQ ID NO: 156 - protein, 120 aa, Artificial Sequence
  synthetic sequence
QVQLVQSGAEVKKPGASVKVSCKASGGTFSNYAISWVRQAPGQGLEWMGWMNPNSGNTGYAQKFQGRVTMTRDTSTSTVYMELSSLRSEDTAVYYCARVNYYYYYGMDVWGQGTTVTVSS

SEQ ID NO: 157 - protein, 107 aa, Artificial Sequence
  synthetic sequence
DIQMTQSPSSLSASVGDRVTITCQASQDISNYLNWYQQKPGKAPKLLIYKASTLKSGVPSRFSGSGSGTDFTLTISSLQPEDFATYYCQQADNLPFTFGPGTKVDIK

SEQ ID NO: 158 - protein, 9 aa, Artificial Sequence
  synthetic sequence
GTFSNYAIS

SEQ ID NO: 159 - protein, 15 aa, Artificial Sequence
  synthetic sequence
CARVNYYYYYGMDVW

SEQ ID NO: 160 - protein, 11 aa, Artificial Sequence
  synthetic sequence
QASQDISNYLN

SEQ ID NO: 161 - protein, 5 aa, Artificial Sequence
  synthetic sequence
STLKS

SEQ ID NO: 162 - protein, 9 aa, Artificial Sequence
  synthetic sequence
QQADNLPFT

SEQ ID NO: 163 - protein, 122 aa, Artificial Sequence
  synthetic sequence
QVQLVQSGAEVKKPGASVKVSCKASGYTFTNYYMHWVRQAPGQGLEWMGIINPSGGSTSYAQRFQGRVTMTRDTSTSTVYMELSSLRSEDTAVYYCARDWGWDYYYYGMDVWGQGTTVTVSS

SEQ ID NO: 164 - protein, 107 aa, Artificial Sequence
  synthetic sequence
DIQMTQSPSSLSASVGDRVTITCQASRDISNYLNWYQQKPGKAPKLLIYAASSLQSGVPSRFSGSGSGTDFTLTISSLQPEDFATYYCQQANSFPPTFGQGTKLEIK

SEQ ID NO: 165 - protein, 9 aa, Artificial Sequence
  synthetic sequence
YTFTNYYMH

SEQ ID NO: 166 - protein, 17 aa, Artificial Sequence
  synthetic sequence
CARDWGWDYYYYGMDVW

SEQ ID NO: 167 - protein, 11 aa, Artificial Sequence
  synthetic sequence
QASRDISNYLN

SEQ ID NO: 168 - protein, 9 aa, Artificial Sequence
  synthetic sequence
QQANSFPPT

SEQ ID NO: 169 - protein, 119 aa, Artificial Sequence
  synthetic sequence
EVQLLESGGGLVQPGGSLRLSCAASGFTFSNSDMSWVRQAPGKGLEWVSGITISGGSTYYADSVRGRFTISRDNSKNTLYLQMSSLRAEDTAVYYCARGRGGSGWLDYWGQGTLVTVSS

SEQ ID NO: 170 - protein, 113 aa, Artificial Sequence
  synthetic sequence
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYTTPPTFGQGTRLEIK

SEQ ID NO: 171 - protein, 9 aa, Artificial Sequence
  synthetic sequence
FTFSNSDMS

SEQ ID NO: 172 - protein, 13 aa, Artificial Sequence
  synthetic sequence
SGITISGGSTYYA

SEQ ID NO: 173 - protein, 14 aa, Artificial Sequence
  synthetic sequence
CARGRGGSGWLDYW

SEQ ID NO: 174 - protein, 17 aa, Artificial Sequence
  synthetic sequence
KSSQSVLYSPNNKNYLA

SEQ ID NO: 175 - protein, 5 aa, Artificial Sequence
  synthetic sequence
STRES

SEQ ID NO: 176 - protein, 9 aa, Artificial Sequence
  synthetic sequence
QQYYTTPPT

SEQ ID NO: 177 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTLTISSLQPEDFATYYCQQANSFPVTFGGGTKVDIK

SEQ ID NO: 178 - protein, 107 aa, Artificial Sequence
  synthetic sequence
DIQMTQSPSSLSASVGDRVTITCRASQSISRWLAWYQQKPGKAPKLLIYGASSLQSGVPSRFSGSGSGTDFTFTISSLQPEDFATYYCQQANSFPVTFGGGTKVDIK

SEQ ID NO: 179 - protein, 120 aa, Artificial Sequence
  synthetic sequence
QVQLVQSGAEVKKPGSSVKVSCKASGGDFSNYFVSWVRQAPGQGLEWMGWINPHNGDTMYAQKFQGRVTITADESTSTAYMELSSLRSEDTAVYYCARGGYSYGYTFDIWGQGTMVTVSS

SEQ ID NO: 180 - protein, 107 aa, Artificial Sequence
  synthetic sequence
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGGGTKVEIK

SEQ ID NO: 181 - protein, 9 aa, Artificial Sequence
  synthetic sequence
GDFSNYFVS

SEQ ID NO: 182 - protein, 13 aa, Artificial Sequence
  synthetic sequence
GWINPHNGDTMYA

SEQ ID NO: 183 - protein, 15 aa, Artificial Sequence
  synthetic sequence
CARGGYSYGYTFDIW

SEQ ID NO: 184 - protein, 11 aa, Artificial Sequence
  synthetic sequence
RASQSISTWLA

SEQ ID NO: 185 - protein, 119 aa, Artificial Sequence
  synthetic sequence
EVQLLESGGGLVQPGGSLRLSCAASGFTFSNSDMSWVRQAPGKGLEWVSGITISGGSTYYADSVKGRFTISRDNSKNTLYLQMNSLRAEDTAVYYCARGRGGSGWLDYWGQGTLVTVSS

SEQ ID NO: 186 - protein, 113 aa, Artificial Sequence
  synthetic sequence
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYITPPTFGQGTRLEIK

SEQ ID NO: 187 - protein, 9 aa, Artificial Sequence
  synthetic sequence
QQYYITPPT

SEQ ID NO: 188 - protein, 113 aa, Artificial Sequence
  synthetic sequence
QVQLVQSGAEVKKPGSSVKVSCKASGHTFTDYYMHWVRQAPGQGLEWMGIINPSGGSTSYAQKFQGRVTITADESTSTAYMELSSLRSEDTAVYYCASGWTDWGQGTLVTVSS

SEQ ID NO: 189 - protein, 108 aa, Artificial Sequence
  synthetic sequence
EIVMTQSPATLSVSPGERATLSCRASQSVSSYLAWYQQKPGQAPRLLIYGTSSRATGIPARFSGSGSGTEFTLTISSLQSEDFAVYYCQQYTTSPITFGQGTRLEIKR

SEQ ID NO: 190 - protein, 9 aa, Artificial Sequence
  synthetic sequence
HTFTDYYMH

SEQ ID NO: 191 - protein, 8 aa, Artificial Sequence
  synthetic sequence
CASGWTDW

SEQ ID NO: 192 - protein, 11 aa, Artificial Sequence
  synthetic sequence
RASQSVSSYLA

SEQ ID NO: 193 - protein, 5 aa, Artificial Sequence
  synthetic sequence
SSRAT

SEQ ID NO: 194 - protein, 9 aa, Artificial Sequence
  synthetic sequence
QQYTTSPIT

SEQ ID NO: 195 - protein, 117 aa, Artificial Sequence
  synthetic sequence
QVQLVQSGAEVKKPGASVKVSCKASGYTFTDYYMHWVRQAPGQGLEWMGGIFPVFGSSTYAQKFQGRVTMTRDTSTSTVYMELSSLRSEDTAVYYCARDHGSGLDVWGQGTTVTVSS

SEQ ID NO: 196 - protein, 107 aa, Artificial Sequence
  synthetic sequence
DIQMTQSPSSLSASVGDRVTITCQASQDISNYLNWYQQKPGKAPKLLIYDAKDLHPGVPSRFSGSGSGTDFTLTISSLQPEDFATYYCQESFSTLTFGQGTKVEIKR

SEQ ID NO: 197 - protein, 9 aa, Artificial Sequence
  synthetic sequence
YTFTDYYMH

SEQ ID NO: 198 - protein, 13 aa, Artificial Sequence
  synthetic sequence
GGIFPVFGSSTYA

SEQ ID NO: 199 - protein, 12 aa, Artificial Sequence
  synthetic sequence
CARDHGSGLDVW

SEQ ID NO: 200 - protein, 5 aa, Artificial Sequence
  synthetic sequence
KDLHP

SEQ ID NO: 201 - protein, 8 aa, Artificial Sequence
  synthetic sequence
QESFSTLT

SEQ ID NO: 202 - protein, 113 aa, Artificial Sequence
  synthetic sequence
QVQLVQSGAEVKKPGASVKVSCKASGYSFTTYYMHWVRQAPGQGLEWMGIIAPSGGSTSYAQKFQGRVTMTRDTSTSTVYMELSSLRSEDTAVYYCASGWVYWGQGTLVTVSS

SEQ ID NO: 203 - protein, 13 aa, Artificial Sequence
  synthetic sequence
GIIAPSGGSTSYA

SEQ ID NO: 204 - protein, 113 aa, Artificial Sequence
  synthetic sequence
QVQLVQSGAEVKKPGASVKVSCKASGYSFTTYYMHWVRQAPGQGLEWMGIIGPSGGSTSYAQKFQGRVTMTRDTSTSTVYMELSSLRSEDTAVYYCASGWVYWGQGTLVTVSS

SEQ ID NO: 205 - protein, 125 aa, Artificial Sequence
  synthetic sequence
QVQLVQSGAEVKKPGASVKVSCKASGYTFSDHYMHWVRQAPGQGLEWMGTIAPSGGRTSYAQKFQGRVTMTRDTSTSTVYMELSSLRSEDTAVYYCAADNGHASGWLYYYGMDVWGQGTTVTVSS

SEQ ID NO: 206 - protein, 13 aa, Artificial Sequence
  synthetic sequence
GTIAPSGGRTSYA

SEQ ID NO: 207 - protein, 125 aa, Artificial Sequence
  synthetic sequence
QVQLVQSGAEVKKPGASVKVSCKASGYTFSDHYMHWVRQAPGQGLEWMGTIGPSGGRTSYAQKFQGRVTMTRDTSTSTVYMELSSLRSEDTAVYYCAADNGHASGWLYYYGMDVWGQGTTVTVSS

SEQ ID NO: 208 - protein, 7 aa, Artificial Sequence
  synthetic sequence
DASSLES

SEQ ID NO: 209 - protein, 5 aa, Artificial Sequence
  synthetic sequence
DYAMH

SEQ ID NO: 210 - protein, 17 aa, Artificial Sequence
  synthetic sequence
WLNPNSGNTGYAPKFQG

SEQ ID NO: 211 - protein, 6 aa, Artificial Sequence
  synthetic sequence
AAGLFI

SEQ ID NO: 212 - protein, 6 aa, Artificial Sequence
  synthetic sequence
QSINRW

SEQ ID NO: 213 - protein, 8 aa, Artificial Sequence
  synthetic sequence
GYTFTDYA

SEQ ID NO: 214 - protein, 8 aa, Artificial Sequence
  synthetic sequence
LNPNSGNT

SEQ ID NO: 215 - protein, 7 aa, Artificial Sequence
  synthetic sequence
GYTFTDY

SEQ ID NO: 216 - protein, 6 aa, Artificial Sequence
  synthetic sequence
NPNSGN

SEQ ID NO: 217 - protein, 7 aa, Artificial Sequence
  synthetic sequence
RASTLQS

SEQ ID NO: 218 - protein, 5 aa, Artificial Sequence
  synthetic sequence
DHYMH

SEQ ID NO: 219 - protein, 17 aa, Artificial Sequence
  synthetic sequence
TINPSGGRTSYAQKFQG

SEQ ID NO: 220 - protein, 18 aa, Artificial Sequence
  synthetic sequence
AADNGHASGWLYYYGMDV

SEQ ID NO: 221 - protein, 6 aa, Artificial Sequence
  synthetic sequence
QSISNW

SEQ ID NO: 222 - protein, 8 aa, Artificial Sequence
  synthetic sequence
GYTFSDHY

SEQ ID NO: 223 - protein, 8 aa, Artificial Sequence
  synthetic sequence
INPSGGRT

SEQ ID NO: 224 - protein, 7 aa, Artificial Sequence
  synthetic sequence
GYTFSDH

SEQ ID NO: 225 - protein, 6 aa, Artificial Sequence
  synthetic sequence
NPSGGR

SEQ ID NO: 226 - protein, 14 aa, Artificial Sequence
  synthetic sequence
NGHASGWLYYYGMD

SEQ ID NO: 227 - protein, 7 aa, Artificial Sequence
  synthetic sequence
WASTRES

SEQ ID NO: 228 - protein, 5 aa, Artificial Sequence
  synthetic sequence
NSDMS

SEQ ID NO: 229 - protein, 17 aa, Artificial Sequence
  synthetic sequence
GITISGGSTYYADSVKG

SEQ ID NO: 230 - protein, 12 aa, Artificial Sequence
  synthetic sequence
ARGRGGSGWLDY

SEQ ID NO: 231 - protein, 12 aa, Artificial Sequence
  synthetic sequence
QSVLYSPNNKNY

SEQ ID NO: 232 - protein, 8 aa, Artificial Sequence
  synthetic sequence
GFTFSNSD

SEQ ID NO: 233 - protein, 8 aa, Artificial Sequence
  synthetic sequence
ITISGGST

SEQ ID NO: 234 - protein, 7 aa, Artificial Sequence
  synthetic sequence
GFTFSNS

SEQ ID NO: 235 - protein, 6 aa, Artificial Sequence
  synthetic sequence
TISGGS

SEQ ID NO: 236 - protein, 8 aa, Artificial Sequence
  synthetic sequence
RGGSGWLD

SEQ ID NO: 237 - protein, 7 aa, Artificial Sequence
  synthetic sequence
AASSLQS

SEQ ID NO: 238 - protein, 5 aa, Artificial Sequence
  synthetic sequence
TYYMH

SEQ ID NO: 239 - protein, 17 aa, Artificial Sequence
  synthetic sequence
IINPSGGSTSYAQKFQG

SEQ ID NO: 240 - protein, 6 aa, Artificial Sequence
  synthetic sequence
ASGWVY

SEQ ID NO: 241 - protein, 6 aa, Artificial Sequence
  synthetic sequence
RDIKNY

SEQ ID NO: 242 - protein, 8 aa, Artificial Sequence
  synthetic sequence
GYSFTTYY

SEQ ID NO: 243 - protein, 8 aa, Artificial Sequence
  synthetic sequence
INPSGGST

SEQ ID NO: 244 - protein, 7 aa, Artificial Sequence
  synthetic sequence
GYSFTTY

SEQ ID NO: 245 - protein, 6 aa, Artificial Sequence
  synthetic sequence
NPSGGS

SEQ ID NO: 246 - protein, 17 aa, Artificial Sequence
  synthetic sequence
GITISGGSTYYADSVRG

SEQ ID NO: 247 - protein, 5 aa, Artificial Sequence
  synthetic sequence
DDYIH

SEQ ID NO: 248 - protein, 17 aa, Artificial Sequence
  synthetic sequence
WMNTNSGNTGYAQKFQG

SEQ ID NO: 249 - protein, 22 aa, Artificial Sequence
  synthetic sequence
ARGGSYSSGWYGRLDYYYGMDV

SEQ ID NO: 250 - protein, 6 aa, Artificial Sequence
  synthetic sequence
QGVGNA

SEQ ID NO: 251 - protein, 8 aa, Artificial Sequence
  synthetic sequence
GYTFTDDY

SEQ ID NO: 252 - protein, 8 aa, Artificial Sequence
  synthetic sequence
MNTNSGNT

SEQ ID NO: 253 - protein, 7 aa, Artificial Sequence
  synthetic sequence
GYTFTDD

SEQ ID NO: 254 - protein, 6 aa, Artificial Sequence
  synthetic sequence
NTNSGN

SEQ ID NO: 255 - protein, 18 aa, Artificial Sequence
  synthetic sequence
GSYSSGWYGRLDYYYGMD

SEQ ID NO: 256 - protein, 113 aa, Artificial Sequence
  synthetic sequence
EVQLVQSGAEVKKPGASVKVSCKASGYSFTSYYMHWVRQAPGQGLEWMGIINPSGGSTSYAQKFQGRVTMTRDTSTSTVYMELSSLRSEDTAVYYCASGWVYWGQGTLVTVSS

SEQ ID NO: 257 - protein, 119 aa, Artificial Sequence
  synthetic sequence
EVQLLESGGGLVQPGGSLRLSCAASGFTFSSYDMSWVRQAPGKGLEWVSGITISGGSTYYADSVKGRFTISRDNSKNTLYLQMNSLRAEDTAVYYCARGRGGSGWLDYWGQGTTVTVSS

SEQ ID NO: 258 - protein, 15 aa, Artificial Sequence
  synthetic sequence
GGGSKGGGSKGGGSK

SEQ ID NO: 259 - protein, 107 aa, Artificial Sequence
  synthetic sequence
DIQMTQSPSSLSASVGDRVTITCRASQSISSYLAWYQQKPGKAPKLLIYAASSLQSGVPSRFSGSGSGTDFTLTISSLQPEDFATYYCQQSYSTPPTFGPGTKVDIK

SEQ ID NO: 260 - protein, 15 aa, Artificial Sequence
  synthetic sequence
AEEEKAEEEKAEEEK

SEQ ID NO: 261 - protein, 107 aa, Artificial Sequence
  synthetic sequence
DIQMTQSPSSLSASVGDRVTITCQASRDIKNYLAWYQQKPGKAPKLLIYAASSLQSGVPSRFSGSGSGTDFTLTISSLQPEDFATYYCQQSYSTPPTFGCGTKVDIK

SEQ ID NO: 262 - protein, 113 aa, Artificial Sequence
  synthetic sequence
EVQLVQSGAEVKKPGASVKVSCKASGYSFTTYYMHWVRQAPGQCLEWMGIINPSGGSTSYAQKFQGRVTMTRDTSTSTVYMELSSLRSEDTAVYYCASGWVYWGQGTLVTVSS

SEQ ID NO: 263 - protein, 113 aa, Artificial Sequence; identical to SEQ ID NO: 170
  synthetic sequence

SEQ ID NO: 264 - protein, 17 aa, Artificial Sequence
  synthetic sequence
IIAPSGGSTSYAQKFQG

SEQ ID NO: 265 - protein, 8 aa, Artificial Sequence
  synthetic sequence
IAPSGGST

SEQ ID NO: 266 - protein, 6 aa, Artificial Sequence
  synthetic sequence
APSGGS

SEQ ID NO: 267 - protein, 5 aa, Artificial Sequence
  synthetic sequence
SYDMS

SEQ ID NO: 268 - protein, 8 aa, Artificial Sequence
  synthetic sequence
GFTFSSYD

SEQ ID NO: 269 - protein, 7 aa, Artificial Sequence
  synthetic sequence
ITISGGS

SEQ ID NO: 270 - protein, 7 aa, Artificial Sequence
  synthetic sequence
GFTFSSY

SEQ ID NO: 271 - protein, 6 aa, Artificial Sequence
  synthetic sequence
QSISSY

SEQ ID NO: 272 - protein, 8 aa, Artificial Sequence
  synthetic sequence
GYSFTSYY

SEQ ID NO: 273 - protein, 7 aa, Artificial Sequence
  synthetic sequence
GYSFTSY

SEQ ID NO: 274 - protein, 107 aa, Artificial Sequence [sequence omitted]

SEQ ID NO: 275 - protein, 113 aa, Artificial Sequence
  synthetic sequence
EVQLVQSGAEVKKPGASVKVSCKASGYSFTTYYMHWVRQAPGQGLEWMGIINPSGGSTSYAQKFQGRVTMTRDTSTSTVYMELSSLRSEDTAVYYCASGWVYWGQGTLVTVSS

SEQ ID NO: 276 - protein, 11 aa, Artificial Sequence
  synthetic sequence
RASQSISSYLA

SEQ ID NO: 277 - protein, 15 aa, Artificial Sequence
  synthetic sequence
AAAEKAAAEKAAAEK

SEQ ID NO: 278 - protein, 119 aa, Artificial Sequence
  synthetic sequence
EVQLLESGGGLVQPGGSLRLSCAASGFTFSSYDMSWVRQAPGKCLEWVSGITISGGSTYYADSVKGRFTISRDNSKNTLYLQMNSLRAEDTAVYYCARGRGGSGWLDYWGQGTTVTVSS

SEQ ID NO: 279 - protein, 113 aa, Artificial Sequence
  synthetic sequence
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYTTPPTFGCGTRLEIK

SEQ ID NO: 280 - protein, 107 aa, Artificial Sequence
  synthetic sequence
DIQMTQSPSSLSASVGDRVTITCRASQSISSYLAWYQQKPGKAPKLLIYAASSLQSGVPSRFSGSGSGTDFTLTISSLQPEDFATYYCQQSYSTPPTFGCGTKVDIK

SEQ ID NO: 281 - protein, 113 aa, Artificial Sequence
  synthetic sequence
EVQLVQSGAEVKKPGASVKVSCKASGYSFTSYYMHWVRQAPGQCLEWMGIINPSGGSTSYAQKFQGRVTMTRDTSTSTVYMELSSLRSEDTAVYYCASGWVYWGQGTLVTVSS

SEQ ID NO: 282 - protein, 107 aa, Artificial Sequence
  synthetic sequence
DIQMTQSPSSLSASVGDRVTITCRASQSISSYLAWYQQKPGAAPKLLIYAASSLQSGVPSRFSGSGSGTDFTLTISSLQPEDFATYYCQQSYSTPPTFGCGTKVDIK

SEQ ID NO: 283 - protein, 107 aa, Artificial Sequence
  synthetic sequence
DIQMTQSPSSLSASVGDRVTITCQASRDIKNYLAWYQQKPGSAPKLLIYAASSLQSGVPSRFSGSGSGTDFTLTISSLQPEDFATYYCQQSYSTPPTFGCGTKVDIK

SEQ ID NO: 284 - protein, 107 aa, Artificial Sequence
  synthetic sequence
DIQMTQSPSSLSASVGDRVTITCRASQSISSYLAWYQQKPGNAPKLLIYAASSLQSGVPSRFSGSGSGTDFTLTISSLQPEDFATYYCQQSYSTPPTFGCGTKVDIK

SEQ ID NO: 285 - protein, 107 aa, Artificial Sequence
  synthetic sequence
DIQMTQSPSSLSASVGDRVTITCQASRDIKNYLAWYQQKPGNAPKLLIYAASSLQSGVPSRFSGSGSGTDFTLTISSLQPEDFATYYCQQSYSTPPTFGCGTKVDIK

SEQ ID NO: 286 - protein, 107 aa, Artificial Sequence
  synthetic sequence
DIQMTQSPSSLSASVGDRVTITCQASRDILNYLAWYQQKPGKAPKLLIYAASSLQSGVPSRFSGSGSGTDFTLTISSLQPEDFATYYCQQSYSTPPTFGCGTKVDIK

SEQ ID NO: 287 - protein, 107 aa, Artificial Sequence
  synthetic sequence
DIQMTQSPSSLSASVGDTVTITCRASQSISSYLAWYQQKPGKAPKLLIYAASSLQSGVPSRFSGSGSGTDFTLTISSLQPEDFATYYCQQSYSTPPTFGCGTKVDIK

SEQ ID NO: 288 - protein, 107 aa, Artificial Sequence
  synthetic sequence
DIQMTQSPSSLSASVGDRVTITCQASRDIKNYLAWYQQKPGAAPKLLIYAASSLQSGVPSRFSGSGSGTDFTLTISSLQPEDFATYYCQQSYSTPPTFGCGTKVDIK

SEQ ID NO: 289 - protein, 107 aa, Artificial Sequence
  synthetic sequence
DIQMTQSPSSLSASVGDTVTITCQASRDIKNYLAWYQQKPGKAPKLLIYAASSLQSGVPSRFSGSGSGTDFTLTISSLQPEDFATYYCQQSYSTPPTFGCGTKVDIK

SEQ ID NO: 290 - protein, 107 aa, Artificial Sequence
  synthetic sequence
DIQMTQSPSSLSASVGDRVTITCRASQSISSYLAWYQQKPGSAPKLLIYAASSLQSGVPSRFSGSGSGTDFTLTISSLQPEDFATYYCQQSYSTPPTFGCGTKVDIK

SEQ ID NO: 291 - protein, 113 aa, Artificial Sequence
  synthetic sequence
EVQLVQSGAEVKKPGASVKVSCKASGYSFTTYYMHWVRQAPGQCLEWMGIINPSGGSTSYAQKFQGRVTMTRDTSTSTVYMELSSLNSEDTAVYYCASGWVYWGQGTLVTVSS

SEQ ID NO: 292 - protein, 107 aa, Artificial Sequence
  synthetic sequence
DIQMTQSPSSLSASVGDRVTITCQASRDINNYLAWYQQKPGKAPKLLIYAASSLQSGVPSRFSGSGSGTDFTLTISSLQPEDFATYYCQQSYSTPPTFGCGTKVDIK

SEQ ID NO: 293 - protein, 113 aa, Artificial Sequence
  synthetic sequence
EVQLVQSGAEVKKPGASVKVSCKASGYSFTSYYMHWVRQAPGQCLEWMGIINPSGGSTSYAQKFQGRVTMTRDTSTSTVYMELSSLNSEDTAVYYCASGWVYWGQGTLVTVSS

SEQ ID NO: 294 - protein, 107 aa, Artificial Sequence
  synthetic sequence
DIQMTQSPSSLSASVGDRVTITCRASQSILSYLAWYQQKPGKAPKLLIYAASSLQSGVPSRFSGSGSGTDFTLTISSLQPEDFATYYCQQSYSTPPTFGCGTKVDIK

SEQ ID NO: 295 - protein, 107 aa, Artificial Sequence
  synthetic sequence
DIQMTQSPSSLSASVGDRVTITCRASQSINSYLAWYQQKPGKAPKLLIYAASSLQSGVPSRFSGSGSGTDFTLTISSLQPEDFATYYCQQSYSTPPTFGCGTKVDIK

SEQ ID NO: 296 - protein, 113 aa, Artificial Sequence
  synthetic sequence
QVQLVQSGAEVKKPGASVKVSCKASGYSFTSYYMHWVRQAPGQCLEWMGIINPSGGSTSYAQKFQGRVTMTRDTSTSTVYMELSSLRSEDTAVYYCASGWVYWGQGTLVTVSS

SEQ ID NO: 297 - protein, 113 aa, Artificial Sequence
  synthetic sequence
QVQLVQSGAEVKKPGASVKVSCKASGYSFTTYYMHWVRQAPGQCLEWMGIINPSGGSTSYAQKFQGRVTMTRDTSTSTVYMELSSLRSEDTAVYYCASGWVYWGQGTLVTVSS

SEQ ID NO: 298 - protein, 197 aa, Artificial Sequence
  synthetic sequence
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQR

SEQ ID NO: 299 - protein, 221 aa, Artificial Sequence
  synthetic sequence
EVQLVQSGAEVKKPGASVKVSCKASGYSFTTYYMHWVRQAPGQGLEWMGIINPSGGSTSYAQKFQGRVTMTRDTSTSTVYMELSSLRSEDTAVYYCASGWVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 300 - protein, 5 aa, Artificial Sequence
  synthetic sequence
GGGSE

SEQ ID NO: 301 - protein, 10 aa, Artificial Sequence
  synthetic sequence
GGGSEGGGSE

SEQ ID NO: 302 - protein, 20 aa, Artificial Sequence
  synthetic sequence
GGGSEGGGSEGGGSEGGGSE

SEQ ID NO: 303 - protein, 5 aa, Artificial Sequence
  synthetic sequence
  MISC_FEATURE (1)..(5): n is 1, 2, 3, 4
GGGGS

SEQ ID NO: 304 - protein, 5 aa, Artificial Sequence
  synthetic sequence
  MISC_FEATURE (1)..(5): n is 1, 2, 3, 4
GGGGA

SEQ ID NO: 305 - protein, 5 aa, Artificial Sequence
  synthetic sequence
  MISC_FEATURE (1)..(5): n is 1, 2, 3, 4
GGGSE

SEQ ID NO: 306 - protein, 5 aa, Artificial Sequence
  synthetic sequence
  MISC_FEATURE (1)..(5): n is 1, 2, 3, 4
GGGSK

SEQ ID NO: 307 - protein, 5 aa, Artificial Sequence
  synthetic sequence
  MISC_FEATURE (1)..(5): n is 1, 2, 3, 4
AEEEK

(19) United States
(12) Patent Application Publication
(10) Pub. No.: US 2024/0052034 A1
(43) Pub. Date: Feb. 15, 2024

(54) MULTI-PARATOPIC ANTI-PD-1 ANTIBODIES AND USES THEREOF

(71) Applicant: PANDION OPERATIONS, INC., Watertown, MA (US)

(72) Inventors: Nathan HIGGINSON-SCOTT, Hingham, MA (US); Kevin Lewis OTIPOBY, Ashland, MA (US); Joanne L. VINEY, Belmont, MA (US); Salvatore ALIOTO, Somerville, MA (US); Lindsay J. EDWARDS, Littleton, MA (US)

(73) Assignee: PANDION OPERATIONS, INC., Watertown, MA (US)

(21) Appl. No.: 18/021,272

(22) PCT Filed: Aug. 19, 2021
(86) PCT No.: PCT/US2021/046656

§ 371 Date: Feb. 14, 2023

Related U.S. Application Data
(60) Provisional application No. 63/067,674, filed on Aug. 19, 2020.
(60) Provisional application No. 63/152,691, filed on Feb. 23, 2021.
(60) Provisional application No. 63/175,760, filed on Apr. 16, 2021.

(51) Int. Cl. C07K 16/28; A61P 37/06

(52) U.S. Cl. CPC C07K 16/2818; A61P 37/06; C07K 2317/33; C07K 2317/64; C07K 2317/622; C07K 2317/92; C07K 2317/94; C07K 2317/41; C07K 2317/73; A61K 2039/505

(57) ABSTRACT

Multi-paratopic polypeptides that can be used, for example, to modulate the activity of PD-1.

### CROSS-REFERENCE TO RELATED APPLICATIONS

This application claims priority to U.S. Provisional Application No. 63/067,674, filed Aug. 19, 2020, U.S. Provisional Application No. 63/175,760, filed Apr. 16, 2021, U.S. Provisional Application No. 63/152,691, filed Feb. 23, 2021, each of which is hereby incorporated by reference in its entirety.

This application is also related to U.S. application Ser. No. 16/997,238, filed Aug. 19, 2020, which is hereby incorporated by reference in its entirety.

### FIELD

The embodiments provided herein relate to, for example, paratopic antibodies that can bind to, for example, PD-1.

### BACKGROUND

Instances of unwanted immune responses, e.g., as in the rejection of transplanted tissue or in autoimmune disorders, constitute a major health problem for millions of people across the world. Long-term outcomes for organ transplantation are frequently characterized by chronic rejection, and eventual failure of the transplanted organ. More than twenty autoimmune disorders are known, affecting essentially every organ of the body, and affecting over fifty million people in North America alone. The broadly active immunosuppressive medications used to combat the pathogenic immune response in both scenarios have serious side effects. Accordingly, therapeutics and polypeptides are needed to treat such conditions. The embodiments provided for herein fulfill these needs as well as others.

### SUMMARY

Disclosed herein are polypeptides that can be used, for example, to modulate an immune response. In some embodiments, the immune response is decreased, which can be used to, for example, treat auto-immune conditions or other immune system disorders where it is necessary or helpful to reduce an immune response. Non-limiting examples of such immune disorders are provided herein.

Embodiments disclosed herein are incorporated by reference into this section.

In some embodiments, the polypeptides provided herein bind and agonize, an inhibitory molecule, e.g., an inhibitory immune checkpoint molecule, or otherwise inhibits or reduces the activity of an immune cell, e.g., a cytotoxic T cell, a helper T cell, a regulatory T cell (Treg), a B cell, NK cell, a dendritic cell, e.g. a plasmacytoid dendritic cell, an innate lymphoid cell, e.g. an ILC2, or a myeloid cell, e.g., a neutrophil or macrophage.

In some embodiments, the level of down regulation of an immune cell is greater when therapeutic compound is bound to its target than when therapeutic compound is not bound to its target. In embodiments, the level of down regulation by target bound therapeutic compound is equal to or 1.5-fold, 2-fold, 4-fold, 8-fold or 10-fold greater than what is seen when it is not bound to its target. In embodiments, therapeutic compound does not, or does not significantly down regulate immune cells when it is not bound to target. Thus, indiscriminate or unwanted agonism of an inhibitory receptor, e.g., PD-1, is minimized or eliminated. E.g., when therapeutic compound is bound to an immune cell, but not bound to the targeted moiety, engagement of a inhibitory immune checkpoint molecule by therapeutic compound does not result in down regulation or does not result in substantial down regulation, e.g., the inhibitory receptor on the immune cell to which therapeutic compound is bound, is not clustered or not clustered sufficiently to result in an inhibitory signal sufficient to give down regulation or substantial inhibition of the immune cell.

In embodiments, therapeutic compound, when engaged with a cell surface inhibitory receptor, e.g., PD-1, on an immune cell, does not inhibit, or does not substantially inhibit the ability of the cell surface inhibitory receptor to bind an endogenous ligand. In some embodiments, therapeutic compound can bind to the PD-L1/2 binding site on PD-1. Thus, indiscriminate or unwanted antagonism of an inhibitory receptor, e.g., PD-1, is minimized or eliminated. In embodiments, binding of therapeutic compound to an inhibitory receptor, e.g. PD-1, on an immune cell does not impede, or substantially impede, the ability of the inhibitory receptor to bind a natural ligand, e.g., PD-L1. In embodiments, binding of therapeutic compound to an inhibitory receptor, e.g. PD-1, on an immune cell reduces binding of a natural ligand, e.g., PD-L1, by less than 50, 40, 30, 20, 10, or 5% of what is seen in the absence of therapeutic compound. In some embodiments, the moiety is an antibody that binds to PD-1. In some embodiments, the antibody is a PD-1 agonist. In some embodiments, the antibody is not a PD-1 antagonist in a soluble PD-1 antagonist assay.

In some embodiments, a therapeutic compound is provided as provided herein.

In some embodiments, the polypeptide described herein comprises first and second binding domains that bind to PD-1. In some embodiments, the first and second binding domains comprise a sequence as set forth in PD-1 Antibody Table 4. In some embodiments, the first and second binding domains comprise a sequence as set forth in PD-1 Antibody Table 5. In some embodiments, the first and second binding domains comprise a sequence as set forth in PD-1 Antibody Table 4 and PD-1 Antibody Table 5.

In some embodiments, the polypeptide described herein comprises first, second, third, and fourth binding domains that bind to PD-1. In some embodiments, the binding domains comprise, independently, a sequence as set forth in PD-1 Antibody Table 4. In some embodiments, the domains comprise, independently, a sequence as set forth in PD-1 Antibody Table 5. In some embodiments, the binding domains comprise, independently, a sequence as set forth in PD-1 Antibody Table 4 and PD-1 Antibody Table 5.

In some embodiments, polypeptide comprises a plurality of distinct binding domains that bind to the same target, such as PD-1. In some embodiments, the polypeptide comprises 2 or 4 distinct binding domains that bind to the target. In some embodiments, the polypeptide comprises 4 binding domains that bind to the target. Where the polypeptide comprises antibody or antibody like sequences, the binding domain can also be referred to as an antigen recognition domain or antigen binding domain. In some embodiments, each of the binding domains bind to the same epitope on the target. In some embodiments, two of the binding domains bind to the same epitope on the target. In some embodiments, such as where there are 4 binding domains, 2 binding domains bind to the same epitope and the remaining 2 binding domains bind to a different epitope on the target. In some embodiments, 2 of the target binding domains have identical or nearly identical target binding domains (e.g. CDRs). In some embodiments, each of the domains have identical or nearly identical target binding domains. In some embodiments, the polypeptide comprises at least one binding domain that is different from another binding domain that is present in the polypeptide. This can also be referred to as a polypeptide having a heterogenous set of target binding domains. A polypeptide that has all of the same target binding domains can be said to have a homogenous set of target binding domains. In some embodiments, where the polypeptide is heterogeneous in regards to the target binding domains, the polypeptide can be referred to as being bispecific. The bispecificity can be in reference to the epitopes that the target binding domains bind to, that is, they are different. It can also be referred to as paratopic or multi-paratopic.

In some embodiments, the polypeptides comprise a formula of, from N-terminus to C-terminus:
(R1-Fc-R2)₂,
wherein R1 is the first binding domain,
wherein R2 is the second binding domain,
wherein one of R1 and R2 is a Fab antibody and the other is a scFv Antibody.
wherein R1 and R2 are linked by a linker,
wherein said linker comprises an Fc immunoglobulin constant region, such as IgG1, IgG2, IgG3, or IgG4,
wherein said linker further comprises a G/S or G/A linker, and
wherein the G/S or G/A linker comprises a sequence of (GGGGS)n or (GGGGA)n, or a combination thereof, wherein each n is independently, 1-4.

In some embodiments, the tetrad antibodies have the general formula, from N-terminus to C-terminus:
(R2-Fc-R1)₂,
wherein R1 is the first binding domain,
wherein R2 is the second binding domain,
wherein one of R1 and R2 is a Fab antibody and the other is a scFv Antibody.
wherein R1 and R2 are linked by a linker,
wherein said linker comprises an Fc immunoglobulin constant region, such as IgG1, IgG2, IgG3, or IgG4,
wherein said linker further comprises a G/S or G/A linker, and
wherein the G/S or G/A linker comprises a sequence of (GGGGS)n or (GGGGA)n, or a combination thereof, wherein each n is independently, 1-4.

In some embodiments, the polypeptide comprises a first polypeptide chain and a second polypeptide chain wherein:
the first polypeptide chain has a formula of from N-terminus to C-terminus:
   [VH-A]-[CH1]-[CH2]-[CH3]-[Linker 1]-[VH-B]-[Linker 2]-[VK-B]; or
   [VH-A]-[CH1]-[CH2]-[CH3]-[Linker 1]-[VK-B]-[Linker 2]-[VH-B];
the second polypeptide chain has a formula of from N-terminus to C-terminus:
   [VK-A]-[CK],
      wherein:
      VH-A=Variable heavy chain domain of a PD1 antibody as provided herein;
      VK-A=Variable light domain of a PD1 antibody as provided herein
      VH-B=Variable heavy chain domain of a PD1 antibody as provided herein;
      VK-B=Variable light domain of a PD1 antibody as provided herein
      CH1=Constant heavy domain 1 of human IgG1
      CH2=Constant heavy domain 2 of human IgG1
      CH3=Constant heavy domain 3 of human IgG1
      CK=Constant domain of kappa light chain
      Linker 1 is a glycine/serine or glycine/alanine linker
      Linker 2 is a glycine/serine or glycine/alanine linker,
      wherein VH-A, VK-A, VH-B, and VK-B can be from the same antibody or different.

In some embodiments, the polypeptide comprises a first polypeptide chain and a second polypeptide chain, wherein:
the first polypeptide chain has a formula of from N-terminus to C-terminus:
   [VH-B]-[CH1]-[CH2]-[CH3]-[Linker 1]-[VH-A]-[Linker 2]-[VK-A]; or
   [VH-B]-[CH1]-[CH2]-[CH3]-[Linker 1]-[VK-A]-[Linker 2]-[VH-A];
the second polypeptide chain has a formula of from N-terminus to C-terminus:
   [VK-B]-[CK],
      wherein:
      VH-A=Variable heavy chain domain of a PD1 antibody as provided herein;
      VK-A=Variable light domain of a PD1 antibody as provided herein;
      VH-B=Variable heavy chain domain of a PD1 antibody as provided herein;
      VK-B=Variable light domain of a PD1 antibody as provided herein;
      CH1=Constant heavy domain 1 of human IgG1, such as provided herein;
      CH2=Constant heavy domain 2 of human IgG1, such as provided herein;
      CH3=Constant heavy domain 3 of human IgG1, such as provided herein;
      CK=Constant domain of kappa light chain, such as provided herein;
      Linker 1 is a glycine/serine or glycine/alanine linker
      Linker 2 is a glycine/serine or glycine/alanine linker,
      wherein VH-A, VK-A, VH-B, and VK-B can be from the same antibody or different.

In some embodiments, the PD-1 antibody comprises a sequence as set forth in PD-1 Antibody Table 4. In some embodiments, the PD-1 antibody comprises a sequence as set forth in PD-1 Antibody Table 5. In some embodiments, the PD-1 antibody comprises a sequence as set forth in PD-1 Antibody Table 4 and PD-1 Antibody Table 5.

In some embodiments, the polypeptide comprises 2 first polypeptide chains and 2 second polypeptide chains. Non-limiting examples are provided for herein.

In some embodiments, a polypeptide is provided comprising first and second binding domains that bind to PD-1, first and second binding domains comprise a sequence as set forth in PD-1 Antibody Table 4 or PD-1 Antibody Table 5. In some embodiments, the polypeptide comprises a third and fourth binding domain that bind to PD-1. In some embodiments, the third binding domain is the same as the first binding domain. In some embodiments, the fourth binding domain is the same as the second binding domain.

In some embodiments, the PD-1 antibody that is in a FAb format has a lower affinity for PD-1 as compared to the PD-1 antibody that in the scFv format. In some embodiments, the PD-1 antibody that is in a FAb format has a higher affinity for PD-1 as compared to the PD-1 antibody that in the scFv format.

In some embodiments, methods of treating autoimmune diseases or conditions are provided herein, the methods comprising administering one or more of therapeutic compounds or polypeptides provided herein.

In some embodiments, methods of treating diseases or conditions described herein are provided herein, the methods comprising administering one or more of therapeutic compounds or polypeptides provided herein.

In some embodiments, methods of treating a subject with inflammatory bowel disease are provided, the methods comprising administering a therapeutic compound or polypeptides provided herein to the subject to treat the inflammatory bowel disease. In some embodiments, the subject has Crohn's disease or ulcerative colitis.

In some embodiments, methods of treating a subject with autoimmune hepatitis are provided, the methods comprising administering a therapeutic compound or polypeptides as provided herein to the subject to treat the autoimmune hepatitis.

In some embodiments, methods of treating primary sclerosing cholangitis are provided, the methods comprising administering a therapeutic compound or polypeptides as provided herein to the subject to treat the primary sclerosing cholangitis.

In some embodiments, methods of treating (e.g., reducing) inflammation in the intestine are provided, the methods comprising administering a therapeutic compound or polypeptides as provided herein to the subject to treat the inflammation in the intestine. In some embodiments, the inflammation is in the small intestine. In some embodiments, the inflammation is in the large intestine. In some embodiments, the inflammation is in the bowel or colon.

In some embodiments, methods of treating (e.g., reducing) inflammation in the pancreas are provided, the methods comprising administering a therapeutic compound or polypeptides as provided herein to the subject to treat the inflammation in the pancreas. In some embodiments, the methods treat pancreatitis.

In some embodiments, methods of treating Type 1 diabetes are provided, the methods comprising administering a therapeutic compound or polypeptides as provided herein to the subject to treat the Type 1 diabetes.

In some embodiments, methods of treating a transplant subject are provided, the methods comprising administering a therapeutically effective amount of a therapeutic compound or polypeptides as provided herein to the subject, thereby treating a transplant (recipient) subject.

In some embodiments, methods of treating graft versus host disease (GVHD) in a subject having a transplanted a donor tissue are provided, the methods comprising administering a therapeutically effective amount of a therapeutic compound or polypeptides as provided herein to the subject.

In some embodiments, methods of treating a subject having, or at risk, or elevated risk, for having, an autoimmune disorder are provided, the methods comprising administering a therapeutically effective amount of a therapeutic compound or polypeptides as provided herein, thereby treating the subject.

### BRIEF DESCRIPTION OF THE DRAWINGS

FIG. 1 depicts non-limiting illustration of polypeptides provided herein.

FIG. 2 depicts a non-limiting illustration of polypeptides provided herein.

FIG. 3 depicts a non-limiting illustration of polypeptides provided herein.

### DETAILED DESCRIPTION

As used herein and unless otherwise indicated, the term “about” is intended to mean±10% of the value it modifies. Thus, about 100 means 95 to 105.

As used herein and in the appended claims, the singular forms “a”, “an” and “the” include plural reference unless the context clearly dictates otherwise.

As used herein, the term “animal” includes, but is not limited to, humans and non-human vertebrates such as wild, domestic, and farm animals.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or to a linker sequence, such as the glycine/serine sequences described herein that link the two domains together.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. An result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent.

As used herein, the phrase “integer from X to Y” means any integer that includes the endpoints. For example, the phrase “integer from X to Y” means 1, 2, 3, 4, or 5.

As used herein, the term “mammal” means a rodent (i.e., a mouse, a rat, or a guinea pig), a monkey, a cat, a dog, a cow, a horse, a pig, or a human. In some embodiments, the mammal is a human.

In some embodiments, polypeptides are provided herein. The polypeptides can also be referred to as compounds. In some embodiments, the polypeptides are therapeutic compounds. In some embodiments, therapeutic compound is a protein or a polypeptide, that has multiple chains that interact with one another. The polypeptides can interact with one another through non-covalent interactions or covalent interactions, such as through disulfide bonds or other covalent bonds. Therefore, if an embodiment refers to a therapeutic compound it can also be said to refer to a protein or polypeptide as provided for herein and vice versa as the context dictates.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

The present disclosure provides, for example, molecules that can act as PD-1 agonists. In some embodiments, the agonist is an antibody or polypeptide comprising a plurality of antigen binding domains that bind to PD-1. Without being bound to any particular theory, agonism of PD-1 inhibits T cell activation/signaling and can be accomplished by different mechanisms. For example cross-linking can lead to agonism, bead-bound, functional PD-1 agonists have been described (Akkaya. Ph.D. Thesis: Modulation of the PD-1 pathway by inhibitory antibody superagonists. Christ Church College, Oxford, U K, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems) which is hereby incorporated by reference, which acts as an agonist when soluble (Said et al., 2010, Nat Med) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1), antibodies against PD-1 and uses therefor, which is hereby incorporated by reference, or can be a bispecific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1. In some embodiments, the antibody does not act as an antagonist of PD-1. In some embodiments, the agonist is an antibody as provided for herein.

PD-1 agonism can be measured by any method, such as the methods described in the examples. For example, cells can be constructed that express, including stably express, constructs that include a human PD-1 polypeptide fused to a beta-galactosidase “Enzyme donor” and 2) a SHP-2 polypeptide fused to a beta-galactosidase “Enzyme acceptor.” Without being bound by any theory, when PD-1 is engaged, SHP-2 is recruited to PD-1. The enzyme acceptor and enzyme donor form a fully active beta-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNγ, IL-17) or other markers, such as CD69 activation marker. Thus, PD-1 agonism can be measured by either cytokine production or cell proliferation. Other methods can also be used to measure PD-1 agonism.

PD-1 is Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (e.g., tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein.

In some embodiments, the portions of the molecule that can bind to PD-1 can be physically tethered, covalently or non-covalently, directly or through a linker entity, to one another, e.g., as a member of the same protein molecule in a polypeptide. In some embodiments, this can be referred to as a fusion protein, such that the different binding moieties are fused together through a chemical or peptide linker. Non-limiting examples of such linkers are provided herein. In some embodiments, the effector moieties (can also be referred to as a effector binding/modulating moiety), which can bind to PD-1, are provided in a polypeptide, e.g., such as, but not limited to, a fusion protein, such as separate domains. In some embodiments, the effector binding/modulating moiety, each comprises a single-chain fragment variable (scFv) or a Fab domain. In some embodiments, therapeutic protein molecule, or a nucleic acid, e.g., an mRNA or DNA, encoding therapeutic protein molecule, can be administered to a subject. In some embodiments, the plurality of effector molecule binding/modulating moieties are linked to a third entity, e.g., a carrier, e.g., a polymeric carrier, a dendrimer, or a particle, e.g., a nanoparticle.

Provided herein are methods of inhibiting GVHD by minimizing the ability of donor immune cells, e.g., donor T cells, to mediate immune attack of recipient tissue, with therapeutic compounds disclosed herein.

Also provided herein are methods of treating, e.g., therapeutically treating or prophylactically treating (or preventing), an autoimmune disorder or response in a subject by administration of a therapeutic compound disclosed herein, e.g., to provide modulation of the immune system. In some embodiments, the modulaton is systemic. In some embodiments, the method provides tolerance to, minimization of the rejection of, minimization of immune effector cell mediated damage to, or prolonging a function of, subject tissue. Non-limiting exemplary tissues include, but are not limited to, the pancreas, myelin, salivary glands, synoviocytes, gut, skin, kidney, lungs, and myocytes.

As used herein, the terms “treat,” “treated,” or “treating” in regard to therapeutic treatment wherein the object is to slow down (lessen) an undesired physiological condition, disorder or disease, or obtain beneficial or desired clinical results. For example, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an autoimmune disease/disorder” means an activity that alleviates or ameliorates any of the primary phenomena or secondary symptoms associated with the autoimmune disease/disorder or other condition described herein. The various disease or conditions are provided herein. Therapeutic treatment can also be administered prophylactically to preventing or reduce the disease or condition before the onset.

In some embodiments, administration of therapeutic compound begins after the disorder is apparent. In some embodiments, administration of therapeutic compound, begins prior to onset, or full onset, of the disorder. In some embodiments, administration of therapeutic compound, begins prior to onset, or full onset, of the disorder, e.g., in a subject having the disorder, a high-risk subject, a subject having a biomarker for risk or presence of the disorder, a subject having a family history of the disorder, or other indicator of risk of, or asymptomatic presence of, the disorder. For example, in some embodiments, a subject having islet cell damage but which is not yet diabetic, is treated.

Unless otherwise defined, all technical and scientific terms used herein have the same meaning as commonly understood by one of ordinary skill in the art to which these embodiments belong. Although methods and materials similar or equivalent to those described herein can be used in the practice or testing of the present embodiments, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the embodiments will be apparent from the description and drawings, and from the claims.

### Additional Definitions

Unless defined otherwise, all technical and scientific terms used herein have the same meaning as commonly understood by one of ordinary skill in the art to which the embodiments pertains. In describing and claiming the present embodiments, the following terminology and terminology otherwise referenced throughout the present application will be used according to how it is defined, where a definition is provided.

It is also to be understood that the terminology used herein is for the purpose of describing particular embodiments only, and is not intended to be limiting.

Antibody, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full-length antibodies and antibody fragments, e.g., dAb (domain antibody), single chain, Fab, Fab′, and F(ab′)2 fragments, and single chain variable fragments (scFvs).

The term “antibody” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MID, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference.

Antibody or antibody molecule can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

As used herein, the term “tetrad” when referring to a polypeptide or antibody as described herein, wherein comprises two sets of effector moieties, and wherein said two sets of effector moieties each individually comprise two binding domains, providing a total of 4 binding domains in the polypeptide.

As used herein, the term “monoparatopic” refers to a therapeutic compound, a molecule, or an antibody as described herein, wherein therapeutic compound, a molecule, or an antibody described herein comprises identical binding domains so that they bind to the same epitope. In some embodiments, the polypeptides provided for here are not monoparatopic. For example, in some embodiments, the polypeptides comprise at least two different binding domains that bind to different epitopes. Therefore, in some embodiments, the polypeptide can be referred to as “biparatopic.” As used herein, the term “biparatopic” refers to a therapeutic compound, a molecule, or an antibody as described herein, wherein therapeutic compound, a molecule, or an antibody described herein comprises different binding domains that bind to different epitopes, including epitopes that do not overlap with one another. In some embodiments, the polypeptide binds to only 2 different epitopes. In some embodiments, the polypeptide binds to 3 or 4 different epitopes.

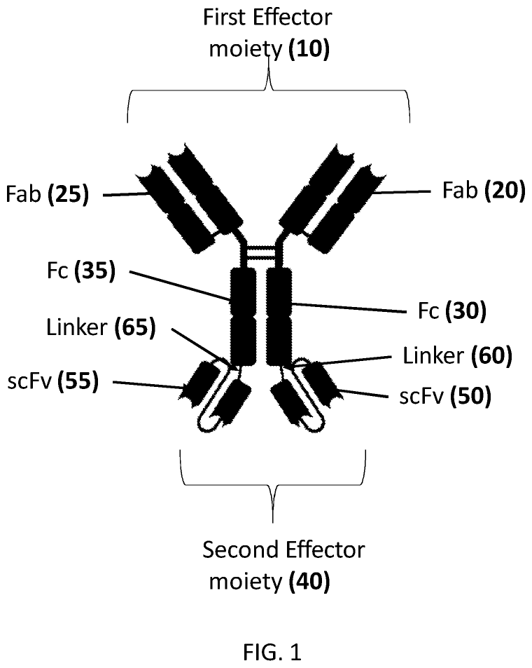

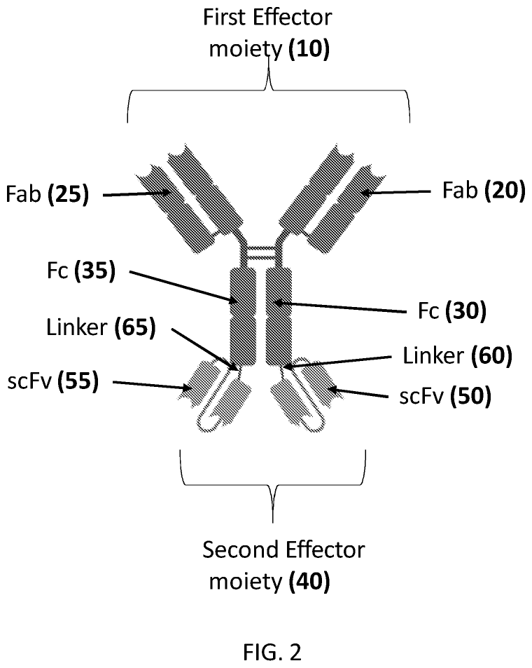

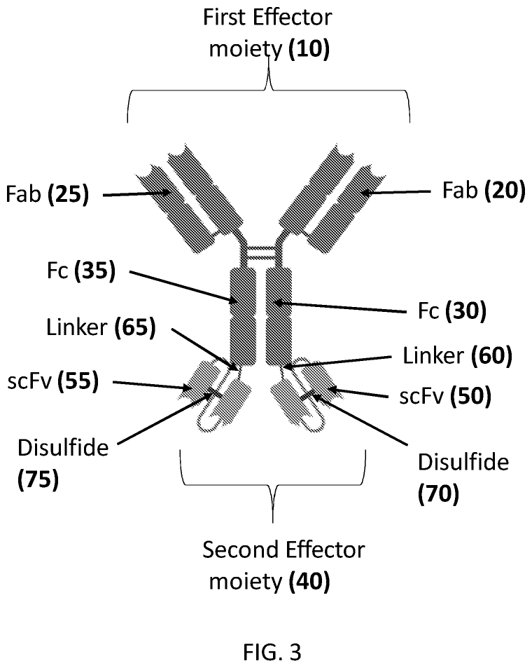

As used herein, “monoparatoric tetrad” or “biparatopic tetrad” refers to a therapeutic compound, a molecule, or an antibody as described herein, wherein therapeutic compound, a molecule, or an antibody described herein comprises two sets of first and second effector moieties that comprise binding domains that bind to a target. Non-limiting examples of such molecules are shown in FIG. 1, FIG. 2, and FIG. 3.

For example, FIG. 1 illustrates a non-limiting configuration of a monoparatopic tetrad molecule as described herein. Referring to FIG. 1, it illustrates a first effector moiety (10) that comprises two Fab portions (domains) (20) and (25), linked to an Fc molecule as described herein (30) and (35), linked via a peptide or chemical linker (60) and (65) to a second effector moiety (40) comprising two scFv portions (50) and (55). In some embodiments, each of the Fab portions (20) and (25) and each of the scFV molecules (50) and (55) bind to the same molecule and to the same epitope. As provided herein, they can bind to PD-1. In some embodiments, the FAb and the scFV comprise the CDRs of PD1AB4, PD1AB25, PD1AB30, PD1AB53, PD1AB37, or any other CDR set as provided for herein.

FIG. 2 illustrates a non-limiting configuration of a biparatopic tetrad molecule as provided for herein. For example, referring to FIG. 2, it illustrates a first effector moiety (10) that comprises two Fab molecules (20) and (25), linked to an Fc molecule as described herein (30) and (35), linked via a peptide or chemical linker (60) and (65) to a second effector moiety (40) comprising two scFv molecules (50) and (55). In some embodiments, each of the Fab portions (domains) (20) and (25) and each of the scFV portions (50) and (55) bind to the same molecule, but to different epitopes. For example, in some embodiments, the FAb (20) and FAb (25) bind to a first epitope on the target molecule and the scFV (50) and (55) bind to a second epitope on the same target molecule. In some embodiments, the epitopes are non-overlapping. In some embodiments, FAb (25) and scFv (55) bind to a first epitope and FAb (20) and scFv (50) bind to a second epitope on the same target molecule. In some embodiments, each of the FAb (20), Fab (25), scFv (50), and scFv (55) bind to different epitopes on the same target molecule. In some embodiments, two of the FAb (20), Fab (25), scFv (50), and scFv (55) bind to a first epitope on the target molecule and the remaining two of FAb (20), Fab (25), scFv (50), and scFv (55) bind to a second epitope on the target molecule. In some embodiments, the target molecule is the same target molecule. In some embodiments, the target molecules are different. When reference is made to the different epitopes throughout the present disclosure, the epitopes can be non-overlapping, i.e. do not share any of the same residues, or they can be overlapping, i.e. they share some of the same residues, but do not bind to the exact same set of residues. As provided herein, in some embodiments, the different epitopes are non-overlapping. As provided herein, the FAb and scFV referenced herein can bind to PD-1. In some embodiments, the FAb and the scFV comprise the CDRs of PD1AB4, PD1AB25, PD1AB30, PD1AB53, PD1AB37, or any other CDR set as provided for herein.

Referencing FIG. 3, in some embodiments, the variable chains in the scFv portion of the molecule are linked (e.g. stabilized) by a disulfide bond (70) and (75). Without being bound to any particular theory, the disulfide bond can help stabilize the scFv formatted binding domain. In some embodiments, the molecule does not comprise one or more of the disulfide bonds linking the domains of the scFv format.

Additionally, referencing FIG. 1, FIG. 2, and FIG. 3 the FAb domains can be linked by a disulfide bond in the hinge region of the polypeptide of chain 1, for example, between the CH1 and CH2 domains.

Effector, as that term is used herein, refers to an entity, e.g., a cell or molecule, e.g., a soluble or cell surface molecule, which mediates an immune response.

Effector ligand binding molecule, as used herein, refers to a polypeptide that has sufficient sequence from a naturally occurring counter ligand of an effector, that it can bind the effector with sufficient specificity that it can serve as an effector binding/modulating molecule. In some embodiments, it binds to effector with at least 10, 20, 30, 40, 50, 60, 70, 80, 90, or 95% of the affinity of the naturally occurring counter ligand. In some embodiments, it has at least 60, 70, 80, 90, 95, 99, or 100% sequence identity, or substantial sequence identity, with a naturally occurring counter ligand for the effector.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally occurring sequence, or are encoded by a substantially identical nucleotide sequence, and are capable of having one or more activities of the naturally occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and) (BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the)(BLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g.,)(BLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Subject, as that term is used herein, refers to a mammalian subject, e.g., a human subject. In some embodiments, the subject is a non-human mammal, e.g., a horse, dog, cat, cow, goat, or pig.

Although specific anti-PD-1 antibodies are provided herein, other anti-PD-1 antibodies can be used. It has been surprisingly found that the tetrad format provided for herein provides the unexpected ability to be a PD-1 agonist at levels that were not predicted and to provide PD-1 agonism. Without being bound to any particular theory, it is thought that tetrad bi-paratopic format provides for greater agonist ability than a monomeric antibody or a tetrad mono-paratopic can provide, which was a surprising result.

The PD-L1/PD-1 Pathway

Programmed cell death protein 1, (often referred to as PD-1) is a cell surface receptor that belongs to the immunoglobulin superfamily. PD-1 is expressed on T cells and other cell types including, but not limited to, B cells, myeloid cells, dendritic cells, monocytes, T regulatory cells, iNK T cells. PD-1 binds two ligands, PD-L1 and PD-L2, and is an inhibitory immune checkpoint molecule. Engagement with a cognate ligand, PD-L1 or PD-L2, in the context of engagement of antigen loaded MHC with the T cell receptor on a T cell minimizes or prevents the activation and function of T cells. The inhibitory effect of PD-1 can include both promoting apoptosis (programmed cell death) in antigen specific T cells in lymph nodes and reducing apoptosis in regulatory T cells (suppressor T cells).

### Auto-Immune Disorders

The polypeptides provided for herein and methods of using the same can be used to treat a subject having, or at risk for having, an unwanted autoimmune response, e.g., an autoimmune response in Type 1 diabetes, multiple sclerosis, cardiomyositis, vitiligo, alopecia, inflammatory bowel disease (IBD, e.g., Crohn's disease or ulcerative colitis), Sjogren's syndrome, focal segmented glomerular sclerosis (FSGS), scleroderma/systemic sclerosis (SSc) or rheumatoid arthritis. In some embodiments, the treatment minimizes rejection of, minimizes immune effector cell mediated damage to, prolongs the survival of subject tissue undergoing, or a risk for, autoimmune attack. In some embodiments, the disorder is Systemic Lupus Erythematosus (SLE).

Other examples of autoimmune disorders and diseases that can be treated with the compounds described herein include, but are not limited to, myocarditis, postmyocardial infarction syndrome, postpericardiotomy syndrome, subacute bacterial endocarditis, anti-glomerular basement membrane nephritis, interstitial cystitis, lupus nephritis, membranous glomerulonephropathy, chronic kidney disease (“CKD”), autoimmune hepatitis, primary biliary cirrhosis, primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (lad), morphea, pemphigus vulgaris, pityriasis lichenoides et varioliformis acuta, Mucha-Habermann disease, psoriasis, systemic scleroderma, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, coeliac disease, Crohn's disease, microscopic colitis, ulcerative colitis, thrombocytopenia, adiposis, dolorosa, adult-onset Still's disease, ankylosing spondylitis, CREST syndrome, drug-induced lupus, enthesitis-related arthritis, eosinophilic fasciitis, Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, rheumatoid arthritis, sarcoidosis, Schnitzler syndrome, systemic lupus erythematosus (SLE), undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (anti-NMDA) receptor encephalitis, Balo concentric sclerosis, Bickerstaff's encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, Oshtoran syndrome, pediatric autoimmune neuropsychiatric disorder associated with streptococcus (PANDAS), progressive inflammatory neuropathy, restless leg syndrome, stiff person syndrome, Sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Behcet's disease, eosinophilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumaticia, vasculitis, primary immune deficiency, and the like.

Other examples of potential autoimmune disorders and diseases, as well as autoimmune comorbidities that can be treated with the compounds described herein include, but are not limited to, chronic fatigue syndrome, complex regional pain syndrome, eosinophilic esophagitis, gastritis, interstitial lung disease, POEMS syndrome, Raynaud's phenomenon, primary immunodeficiency, pyoderma gangrenosum, agammaglobulinemia, amyloidosis, amyotrophic lateral sclerosis, anti-tubular basement membrane nephritis, atopic allergy, atopic dermatitis, autoimmune peripheral neuropathy, Blau syndrome, Castleman's disease, Chagas disease, chronic obstructive pulmonary disease, chronic recurrent multifocal osteomyelitis, complement component 2 deficiency, contact dermatitis, Cushing's syndrome, cutaneous leukocytoclastic angiitis, Degos disease, eczema, eosinophilic gastroenteritis, eosinophilic pneumonia, erythroblastosis fetalis, fibrodysplasia ossificans progressive, gastrointestinal pemphigoid, hypogammaglobulinemia, idiopathic giant cell myocarditis, idiopathic pulmonary fibrosis, IgA nephropathy, immunoregulatory lipoproteins, IPEX syndrome, ligenous conjunctivitis, Majeed syndrome, narcolepsy, Rasmussen's encephalitis, schizophrenia, serum sickness, spondyloathropathy, Sweet's syndrome, Takayasu's arteritis, Duchenne's muscular dystrophy, Becker muscular dystrophy, congenital muscular dystrophy, myotonic dystrophy, facioscapulohumeral (FHSD) muscular dystrophy, limb-girdle muscular dystrophy, oculopharyngeal muscular dystrophy (OPMD), distal muscular dystrophy, Emery-Dreifuss muscular dystrophy, pulmonary arterial hypertension, asthma, chronic rhinosinusitis, hypersensitivity pneumonitis, non-specific interstitial pneumonia, pre-eclampsia, miscarriage, recurrent miscarriage, aplastic anemia, autoimmune neutropenia, autoimmune hemolytic anemia, cancer immunotherapy-associated autoimmune disease, and the like.

In some embodiments, the autoimmune disorder does not comprise pemphigus vulgaris, pemphigus. In some embodiments, the autoimmune disorder does not comprise pemphigus foliaceus. In some embodiments, the autoimmune disorder does not comprise bullous pemphigoid. In some embodiments, the autoimmune disorder does not comprise Goodpasture's disease. In some embodiments, the autoimmune disorder does not comprise psoriasis. In some embodiments, the autoimmune disorder does not comprise a skin disorder. In some embodiments, the disorder does not comprise a neoplastic disorder, e.g., cancer.

Additionally as provided for herein, it has been found that TLR9 activation leads to an increase of PD-1 expression in plasmacytoid dendritic cells. TLR9 activation by CpGA was also found to increase the production of interferon. Although not wishing to be bound to any particular theory, utilizing the PD-1 agonists provided herein led to a decrease in interferon production. Therefore, in some embodiments, the compounds and compositions provided herein can be used to treat intereferonopathies, which was not previously known and a surprising an unexpected result to find that PD-1 can be expressed on the surface of plasmacytoid dendritic cells. Thus, the PD-1-binding biparatopic molecules provided for herein can be used to modulate a TLR9 mediated immune response. Toll-like receptors (TLRs) are essential for innate immune responses as they recognize several different antigens and initiate immunological/inflammatory responses such as cytokine production, and dendritic cell and macrophage activation. Especially, TLR2, TLR3, TLR4, TLR7, TLR8, and TLR9 recognize viral or bacterial ligands such as glycoprotein, single- or double-stranded RNA and polynucleotide containing unmethylated 5′-CG-3′ sequences. Additionally, immunostimulatory nucleic acid molecules stimulate the immune response through interaction with and signaling through the mammalian TLR9 receptor. (Hemmi et al. (2002) Nat. Immunol. 3:196-200) Plasmacytoid dendritic cells (PDCs), a distinct subset of dendritic cells (DCs), are capable of rapidly secreting large amounts of type I interferon (IFN) in response to viral infection through endosomal TLR activation. Without being bound to any particular theory, the triggering of TLR7 and TLR9 in PDCs and B cells by self-nucleic acids is key in the pathogenesis of Systemic Lupus Erythematosus (SLE). This can lead to the production of type I IFN from PDCs that can be detected by the upregulation of IFN-regulated genes in the blood of patients (IFN-signature) and anti-DNA and anti-RNP antibodies from B cells that form immune complexes (IC) with DNA or RNA from dying cells (Barrat and Coffman, 2008; Marshak-Rothstein, 2006). Once activated, PDCs migrate from the blood into inflamed tissues including skin and kidney. IFN and PDC have been proposed to contribute to the pathogenesis of other autoimmune diseases characterized by IFN signature as well. Indeed, Type I IFN-producing PDC accumulate in the pancreas, muscle and salivary glands of people affected by diabetes mellitus, dermatomyositis and Sjogren's syndrome respectively, strongly suggesting that dysregulated PDC activation could be a more general feature of autoimmune disease (Barrat and Coffman, 2008; Guiducci et al., 2009; Ueno et al., 2007).

Without wishing to be bound by a particular theory, the present disclosure finds that TLR9 activation can also lead to induction of PD-1 expression on PDCs. PD-1 agonism can lead to inhibition of TLR9-mediated activation and the effector functions of PDCs. In some embodiments, PD-1 agonism can lead to reduced or no IFN production in PDCs. In some embodiments, the molecules disclosed herein are PD-1 agonists. In some embodiments, the PD-1 agonists of the disclosure can inhibit TLR-9 activity in PDCs. In some embodiments, inhibition of TLR-9 activity mediated by the use PD-1 agonists of the disclosure can lead to reduced or lack of production of IFN in PDCs.

In some embodiments, the PD-1 agonists of the disclosure can be used to treat interferonopathies. In some embodiments, the interferonopathy is a type I interferonopathy. In some embodiments the type I interferonopathy is Aicardi-Goutieres syndrome, bilateral striatal necrosis, chronic atypical neutrophilic dermatosis with lipodystrophy and elevated temperature (CANDLE), complete non-penetrance, dyschromatosis symmetrica hereditaria, familial chilblain lupus, Japanese autoinflammatory syndrome with lipodystrophy (JASL), joint contractures, muscle atrophy, microcytic anaemia, panniculitis, and lipodystrophy (JMP), Mendelian susceptibility to mycobacterial disease (MSMD), Nakajo-Nishimura syndrome, retinal vasculopathy with cerebral leukodystrophy (RVCL), spastic paraparesis, STING-associated vasculopathy with onset in infancy (SAVI), Singleton-Merten syndrome, or spondylochondromatosis (SPENCD). The term interferonopathy, as used herein, is meant to refer to a general pathology of the interferon system, congenital or acquired, which includes the following types of disorders of the IFN system: deficiency; paralysis of the IFN system; inadequate response on viruses, bacteria, and mutated tumor cells; and overproduction of type I IFN. In some embodiments, interferonopathy comprises autoimmune diseases. In some embodiments, the autoimmune disease is Systemic Lupus Erythematosus (SLE).

In some embodiments, the subject being treated for an interferonopathy is a subject in need thereof. That is, the subject is being treated with the compositions and molecules provided for herein with an intent to treat such interferonopathy.

In some embodiments, methods of treating interferonopathies are provided. In some embodiments, the methods comprise administering to a subject, including a subject in need thereof, a molecule or composition as provided for herein. In some embodiments, the method comprises inhibiting the production of interferon from plasmacytoid dendritic cells. In some embodiments, the interferon production is reduced by about, or at least, 10, 20, 30, 40, 50, 60, 70, 80, 90 or 95% as compared to the amount of interferon produced in the absence of the paratopic PD-1 agonists provided for herein.

In some embodiments, methods of reducing the production of interferon are provided. In some embodiments, the methods comprise administering to a subject, including a subject in need thereof, a molecule or composition as provided for herein. In some embodiments, In some embodiments, the interefron production is reduced by about, or at least, 10, 20, 30, 40, 50, 60, 70, 80, 90 or 95% as compared to the amount of interferon produced in the absence of the paratopic PD-1 agonists provided for herein.

In some embodiments, methods of inhibiting TLR9 mediated production of interferon in a subject are provided. In some embodiments, the methods comprise administering to a subject, including a subject in need thereof, a molecule or composition as provided for herein. In some embodiments. In some embodiments, the interefron production is reduced by about, or at least, 10, 20, 30, 40, 50, 60, 70, 80, 90 or 95% as compared to the amount of interferon produced in the absence of the paratopic PD-1 agonists provided for herein.

In some embodiments, method of treating a TLR9 mediated disorder is provided. In some embodiments, the methods comprise administering to a subject, including a subject in need thereof, a molecule or composition as provided for herein. In some embodiments, the TLR9 mediated disorder is a type I interferonopathy. Non-limiting examples of type I interferonopathies are provided for herein.

In some embodiments, methods of inhibiting the upregulation of IFN-regulated genes in the blood of patients (IFN-signature) is provided. In some embodiments, In some embodiments, the methods comprise administering to a subject, including a subject in need thereof, a molecule or composition as provided for herein. In some embodiments, the genes that are inhibited are OAS1, IFIT3, MX1 and/or IFN-β1.

In some embodiments, methods of inhibiting the expression of OAS1, IFIT3, MX1 and IFN-β1 in a cell or a subject are provided. In some embodiments, the methods comprise administering to the subject or contacting a cell with a polypeptide, protein or antibody as provided herein. In some embodiments, the expression of OAS1, IFIT3, MX1 and IFN-β1 is the gene expression. In some embodiments, the cell is a plasmacytoid dendritic cell. In some embodiments, the plasmacytoid dendritic cell is an activated plasmacytoid dendritic cell. In some embodiments, the gene expression as measured by mRNA levels of the genes, is inhibited by, or at least, 10%, 20%, 30%, 40%, 50%, 60%, 70%, 80%, 90% or 95% as compared to the same cell or subject that has not been administered or contacted with polypeptide, protein or antibody as provided herein.

In some embodiments, the PD-1 agonists of the disclosure can be used to treat IgG4 related disease. In some embodiments, the IgG4 related disease is a chronic inflammation. In some embodiments, the IgG4 related disease is a spectrum of complex fibroinflammatory disorder. The polypeptides provided herein can, for example, comprise a plurality of effector binding/modulating moieties. Any suitable linker or platform can be used to present the plurality of moieties. The linker can be typically coupled or fused to one or more effector binding/modulating moieties.

Linker Regions

As discussed elsewhere effector binding/modulating moieties can be linked by linker regions. The linker can be a peptide linker or a chemical linker (e.g. small molecule). Any linker region described herein can be used as a linker. For example, the linkers can comprise Fc regions. This is illustrated, in part, in FIG. 1 and FIG. 2. In some embodiments, a therapeutic compound comprises a Linker that can self-associate. In some embodiments, a therapeutic compound comprises a Linker that has a moiety that minimizes self-association. Linkers also include glycine/serine linkers. In some embodiments, the linker is a glycine/glutamic acid/serine linker. In some embodiment, the linker is alanine/glutamic acid/lysine linker. In some embodiments, the linker is a glycine/alanine linker. In some embodiments, the linker can comprise one or more repeats of GGGGS (SEQ ID NO: 4). In some embodiments, the linker comprises 1, 2, 3, or 4 repeats of SEQ ID NO: 4 or SEQ ID NO: 8. In some embodiments, the linker comprises of GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), or GGGGAGGGGAGGGGAGGGGA (SEQ ID NO: 11). In some embodiments, the linker comprises GGGSEGGGSEGGGSE (SEQ ID NO: 1). In some embodiments, the linker comprises GGGSKGGGSKGGGSK (SEQ ID NO: 258). In some embodiments, the linker comprises AEEEKAEEEKAEEEK (SEQ ID NO: 260). These linkers can be used in any of therapeutic compounds, polypeptides, or compositions provided herein.

The linker region can comprise a Fc region that has been modified (e.g., mutated) to produce a heterodimer. In some embodiments, the CH3 domain of the Fc region can be mutated. Examples of such Fc regions can be found in, for example, U.S. Pat. No. 9,574,010, which is hereby incorporated by reference in its entirety. The Fc region as defined herein comprises a CH3 domain or fragment thereof, and may additionally comprise one or more addition constant region domains, or fragments thereof, including hinge, CHL or CH2. It will be understood that the numbering of the Fc amino acid residues is that of the EU index as in Kabat et al 1991, NIH Publication 91-3242, National Technical Information Service, Springfield, Va. The “EU index as set forth in Kabat” refers to the EU index numbering of the human IgG1 Kabat antibody. For convenience, Table B of U.S. Pat. No. 9,574,010 provides the amino acids numbered according to the EU index as set forth in Kabat of the CH2 and CH3 domain from human IgG1, which is hereby incorporated by reference. Table 1.1 of U.S. Pat. No. 9,574,010 provides mutations of variant Fc heterodimers that can be used as linker regions. Table 1.1 of U.S. Pat. No. 9,574,010 is hereby incorporated by reference.

In some embodiments, the Linker comprises a first CH3 domain polypeptide and/or a second CH3 domain polypeptide, the first and second CH3 domain polypeptides independently comprising amino acid modifications as compared to a wild-type CH3 domain polypeptide, wherein the first CH3 domain polypeptide comprises amino acid modifications at positions T350, L351, F405, and Y407, and the second CH3 domain polypeptide comprises amino acid modifications at positions T350, T366, K392 and T394, wherein the amino acid modification at position T350 is T350V, T3501, T350L or T350M; the amino acid modification at position L351 is L351Y; the amino acid modification at position F405 is F405A, F405V, F405T or F405S; the amino acid modification at position Y407 is Y407V, Y407A or Y4071; the amino acid modification at position T366 is T366L, T366I, T366V, or T366M; the amino acid modification at position K392 is K392F, K392L or K392M; and the amino acid modification at position T394 is T394W, and wherein the numbering of amino acid residues is according to the EU index as set forth in Kabat.

In some embodiments, the amino acid modification at position K392 is K392M or K392L. In some embodiments, the amino acid modification at position T350 is T350V. In some embodiments, the first CH3 domain polypeptide further comprises one or more amino acid modifications selected from Q347R and one of 5400R or S400E. In some embodiments, the second CH3 domain polypeptide further comprises one or more amino acid modifications selected from L351Y, K360E, and one of N390R, N390D or N390E. In some embodiments, the first CH3 domain polypeptide further comprises one or more amino acid modifications selected from Q347R and one of 5400R or S400E, and the second CH3 domain polypeptide further comprises one or more amino acid modifications selected from L351Y, K360E, and one of N390R, N390D or N390E. In some embodiments, the amino acid modification at position T350 is T350V. In some embodiments, the amino acid modification at position F405 is F405A. In some embodiments, the amino acid modification at position Y407 is Y407V. In some embodiments, the amino acid modification at position T366 is T366L or T366I. In some embodiments, the amino acid modification at position F405 is F405A, the amino acid modification at position Y407 is and Y407V, the amino acid modification at position T366 is T366L or T366I, and the amino acid modification at position K392 is K392M or K392L. In some embodiments, the first CH3 domain polypeptide comprises the amino acid modifications T350V, L351Y, S400E, F405V and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405T and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405S and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, L351Y, T366L, N390R, K392M and T394W. In some embodiments, the first CH3 domain polypeptide comprises the amino acid modifications Q347R, T350V, L351Y, S400E, F405A and Y407V, and the second CH3 domain polypeptide comprises the amino acid modifications T350V, K360E, T366L, N390R, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390D, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390E, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392L and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392F and T394W.

In some embodiments, an isolated heteromultimer comprising a heterodimeric CH3 domain comprising a first CH3 domain polypeptide and a second CH3 domain polypeptide, the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y4071; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70° C. or greater and a purity greater than about 90%, and wherein the numbering of amino acid residues is according to the EU index as set forth in Kabat.

In some embodiments, the Linker comprises a first CH3 domain polypeptide and/or a second CH3 domain polypeptide, wherein the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y4071; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70 C. or greater and a purity greater than about 90%, and wherein the numbering of amino acid residues is according to the EU index as set forth in Kabat. In some embodiments, the amino acid modification at position F405 is F405A. In some embodiments, the amino acid modification at position T366 is T366I or T366L. In some embodiments, the amino acid modification at position Y407 is Y407V. In some embodiments, the amino acid modification at position F405 is F405A, the amino acid modification at position Y407 is Y407V, the amino acid modification at position T366 is T366I or T366L, and the amino acid modification at position K392 is K392L or K392M. In some embodiments, the amino acid modification at position F405 is F405A, the amino acid modification at position Y407 is Y407V, the amino acid modification at position T366 is T366L, and the amino acid modification at position K392 is K392M. In some embodiments, the amino acid modification at position F405 is F405A, the amino acid modification at position Y407 is Y407V, the amino acid modification at position T366 is T366L, and the amino acid modification at position K392 is K392L. In some embodiments, the amino acid modification at position F405 is F405A, the amino acid modification at position Y407 is Y407V, the amino acid modification at position T366 is T366I, and the amino acid modification at position K392 is K392M. In some embodiments, the amino acid modification at position F405 is F405A, the amino acid modification at position Y407 is Y407V, the amino acid modification at position T366 is T366I, and the amino acid modification at position K392 is K392L. In some embodiments, the first CH3 domain polypeptide further comprises an amino acid modification at position S400 selected from S400D and S400E, and the second CH3 domain polypeptide further comprises the amino acid modification N390R. In some embodiments, the amino acid modification at position F405 is F405A, the amino acid modification at position Y407 is Y405V, the amino acid modification at position S400 is S400E, the amino acid modification at position T366 is T366L, and the amino acid modification at position K392 is K392M.

In some embodiments, the modified first and second CH3 domains are comprised by an Fc construct based on a type G immunoglobulin (IgG). The IgG can be, for example, an IgG1, IgG2, IgG3, or IgG4.

Other linkers comprising variant CH3 domains are described in U.S. Pat. Nos. 9,499,634 and 9,562,109, each of which is incorporated by reference in its entirety.

In some embodiments, the linkers can be complementary fragments of a protein, e.g., a naturally occurring protein such as human serum albumin. In some embodiments, the segmentation site resides on a loop of the albumin polypeptide that has a high solvent accessible surface area (SASA) and limited contact with the rest of the albumin structure. In some embodiments, the segmentation results in a complementary interface between the transporter polypeptides. These segmentation sites are described, for example, in U.S. Pat. No. 9,388,231, which is hereby incorporated by reference in its entirety.

In some embodiments, the first polypeptide comprises residues 1-337 or residues 1-293 of the albumin polypeptide with one or more of the mutations described herein. In some embodiments, the second polypeptide comprises residues of 342-585 or 304-585 of the albumin polypeptide with one or more of the mutations described herein. In some embodiments, the first polypeptide comprises residues 1-339, 1-300, 1-364, 1-441, 1-83, 1-171, 1-281, 1-293, 1-114, 1-337, or 1-336 of the albumin protein. In some embodiments, the second polypeptide comprises residues 301-585, 365-585, 442-585, 85-585, 172-585, 282-585, or 115-585, 304-585, 340-585, or 342-585 of the albumin protein.

In some embodiments, the first and second polypeptide comprise the residues of the albumin protein as shown in the table below. The sequence of the albumin protein is described below.

| First Polypeptide Residues | Second Polypeptide Residues |
|---|---|
| 1-300 | 301-585 |
| 1-364 | 365-585 |
| 1-441 | 442-585 |
| 1-83 | 85-585 |
| 1-171 | 172-585 |
| 1-281 | 282-585 |
| 1-114 | 115-585 |
| 1-339 | 340-585 |
| 1-337 | 342-585 |
| 1-293 | 304-585 |
| 1-336 | 342-585 |

In some embodiments, the first and second polypeptides comprise a linker that can form a covalent bond with one another, such as a disulfide bond. A non-limiting example of the linker is a peptide linker. In some embodiments, the peptide linker comprises GGGGS (SEQ ID NO: 4). The linker can be fused to the C-terminus of the first polypeptide and the N-terminus of the second polypeptide. The linker can also be used to attach the moieties described herein without abrogating the ability of the linkers to form a disulfide bond. In some embodiments, the first and second polypeptides do not comprise a linker that can form a covalent bond. In some embodiments, the first and second polypeptides have the following substitutions.

| First Polypeptide Substitution | Second Polypeptide Substitution |
|---|---|
| A217C | V343C |
| L331C | A350C |
| A217C | L346C |
| W214C | V343C |
| A335C | L346C |
| L198C | V455C |
| A217C | A335C |
| A217C | L331C |
| L198C | N458C |
| A194C | V455C |

The sequence of the albumin polypeptide can be the sequence of human albumin as shown, in the post-protein form with the N-terminal signaling residues removed

(SEQ ID NO: 3)
(MKWVTFISLLFLFSSAYSRGVFRR)
DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGL
(human albumin, SEQ ID NO: 2)

In some embodiments, the polypeptide comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of therapeutic compound. Thus, in some embodiments, the antibody comprised of F(ab′)2 on an IgG1 Fc backbone can be an anti-PD-1 antibody on an IgG1 Fc or any other effector binding/modulating moiety provided herein. In some embodiments, the scFv segments fused to the C-terminus could be an anti-PD-1 antibody, if the N-terminus region is an anti-PD-1 antibody. In this non-limiting example, the N-terminus can be the effector binding/modulating moiety, such as any one of the ones provided for herein, and the C-terminus can be another effector binding/modulating moiety, such as any of the ones provided for herein. In some embodiments, the effector binding/modulating moiety is the same as another effector binding/modulating moiety. In some embodiments, the effector binding/modulating moiety is different than another effector binding/modulating moiety.

In some embodiments, the polypeptides comprise a formula of, from N-terminus to C-terminus:
(R1-Fc-R2)₂,
wherein R1 is the first binding domain,
wherein R2 is the second binding domain,
wherein one of R1 and R2 is a Fab antibody and the other is a scFv Antibody.
wherein R1 and R2 are linked by a linker,
wherein said linker comprises an Fc immunoglobulin constant region, such as IgG1, IgG2, IgG3, or IgG4,
wherein said linker further comprises a G/S or G/A linker, and
wherein the G/S or G/A linker comprises a sequence of (GGGGS)n or (GGGGA)n, or a combination thereof, wherein each n is independently, 1-4.

In some embodiments, the tetrad antibodies have the general formula, from N-terminus to C-terminus:
(R2-Fc-R1)₂,
wherein R1 is the first binding domain,
wherein R2 is the second binding domain,
wherein one of R1 and R2 is a Fab antibody and the other is a scFv Antibody.
wherein R1 and R2 are linked by a linker,
wherein said linker comprises an Fc immunoglobulin constant region, such as IgG1, IgG2, IgG3, or IgG4,
wherein said linker further comprises a G/S or G/A linker, and
wherein the G/S or G/A linker comprises a sequence of (GGGGS)n or (GGGGA)n, or a combination thereof, wherein each n is independently, 1-4.

In some embodiments, the polypeptides comprise a first polypeptide chain comprising a Fab heavy chain domain linked by a first linker to a scFv antibody and a second polypeptide chain comprising a Fab light (kappa) chain domain, wherein the Fab heavy and light chains bind to PD-1 and the scFv antibody binds to PD-1 at the same or different epitopes. In some embodiments, the first linker comprises an Fc immunoglobulin constant region, such as IgG1, IgG2, IgG3, or IgG4, and further comprises a sequence of (GGGGS)n or (GGGGA)n, or a combination thereof, wherein each n is independently, 1-4. In some embodiments, the scFv comprises heavy chain variable domain linked to a light chain variable domain with a scFv linker, wherein said scFv linker comprises a sequence of (GGGGS)n, (GGGGA)n, (GGGSE)n (GGGSK)n, or (AEEEK)n, or a combination thereof, wherein each n is independently, 1-4.

The sequences of the first linker and the scFv linker, which are independent of one another can be the same or different and as otherwise described throughout the present application. Thus, in some embodiments, the first linker comprises GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), or GGGGAGGGGAGGGGAGGGGA (SEQ ID NO: 11). In some embodiments, the scFv linker comprises GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), or GGGGAGGGGAGGGGAGGGGA (SEQ ID NO: 11), or GGGSEGGGSEGGGSE (SEQ ID NO: 1). In some embodiments, the linker comprises GGGSKGGGSKGGGSK (SEQ ID NO: 258). In some embodiments, the linker comprises AEEEKAEEEKAEEEK (SEQ ID NO: 260).

In some embodiments, the polypeptide comprises a first polypeptide chain and a second polypeptide chain wherein:
the first polypeptide chain has a formula of from N-terminus to C-terminus:
   [VH-A]-[CH1]-[CH2]-[CH3]-[Linker 1]-[VH-B]-[Linker 2]-[VK-B]; or
   [VH-A]-[CH1]-[CH2]-[CH3]-[Linker 1]-[VK-B]-[Linker 2]-[VH-B];
the second polypeptide chain has a formula of from N-terminus to C-terminus:
   [VK-A]-[CK],
      wherein:
VH-A=Variable heavy chain domain of a PD1 antibody as provided herein;
VK-A=Variable light domain of a PD1 antibody as provided herein
VH-B=Variable heavy chain domain of a PD1 antibody as provided herein;
VK-B=Variable light domain of a PD1 antibody as provided herein
CH1=Constant heavy domain 1 of human IgG1
CH2=Constant heavy domain 2 of human IgG1
CH3=Constant heavy domain 3 of human IgG1
CK=Constant domain of kappa light chain
Linker 1 is a glycine/serine or glycine/alanine linker
Linker 2 is a glycine/serine or glycine/alanine linker,
wherein VH-A, VK-A, VH-B, and VK-B can be from the same antibody or different.

In some embodiments, the polypeptide comprises a first polypeptide chain and a second polypeptide chain, wherein:
the first polypeptide chain has a formula of from N-terminus to C-terminus:
   [VH-B]-[CH1]-[CH2]-[CH3]-[Linker 1]-[VH-A]-[Linker 2]-[VK-A]; or
   [VH-B]-[CH1]-[CH2]-[CH3]-[Linker 1]-[VK-A]-[Linker 2]-[VH-A];
the second polypeptide chain has a formula of from N-terminus to C-terminus:
   [VK-B]-[CK],
      wherein:
VH-A=Variable heavy chain domain of a PD1 antibody as provided herein;
VK-A=Variable light domain of a PD1 antibody as provided herein;
VH-B=Variable heavy chain domain of a PD1 antibody as provided herein;
VK-B=Variable light domain of a PD1 antibody as provided herein;
CH1=Constant heavy domain 1 of human IgG1, such as provided herein;
CH2=Constant heavy domain 2 of human IgG1, such as provided herein;
CH3=Constant heavy domain 3 of human IgG1, such as provided herein;
CK=Constant domain of kappa light chain, such as provided herein;
Linker 1 is a glycine/serine, glycine/glutamic acid/serine, alanine/glutamic acid/lysine, or glycine/alanine linker, or other linkers as provided for herein;
Linker 2 is a glycine/serine, glycine/glutamic acid/serine, alanine/glutamic acid/lysine, or glycine/alanine linker, or other linkers as provided for herein;
wherein VH-A, VK-A, VH-B, and VK-B can be from the same antibody or different.

In some embodiments, CH1, CH2, and CH3 are the domains from the IgG Fc region. The sequence of CH1-CH2-CH3 can be, for example:

(SEQ ID NO: 12)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG

In some embodiments, Linker 1 comprises 1, 2, 3, or 4 GGGGS (SEQ ID NO: 4) and/or GGGGA (SEQ ID NO: 8) and/or GGGSE (SEQ ID NO: 300) repeats. In some embodiments, Linker 2 comprises 1, 2, 3, or 4 GGGGS (SEQ ID NO: 4), GGGSE (SEQ ID NO: 300), and/or GGGGA (SEQ ID NO: 8) repeats. For the avoidance of doubt, the sequences of Linker 1 and Linker 2, which are used throughout this application, are independent of one another. Therefore, in some embodiments, Linker 1 and Linker 2 can be the same or different. In some embodiments, Linker 1 comprises GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), or GGGGAGGGGAGGGGAGGGGA (SEQ ID NO: 11). In some embodiments, Linker 2 comprises GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), GGGGAGGGGAGGGGAGGGGA (SEQ ID NO: 11), GGGSE (SEQ ID NO: 300), GGGSEGGGSE (SEQ ID NO: 301), GGGSEGGGSEGGGSE (SEQ ID NO: 1), or GGGSEGGGSEGGGSEGGGSE (SEQ ID NO: 302).

In some embodiments, the polypeptide comprises a light chain and a heavy chain. In some embodiments, the light and heavy chain begin at the N-terminus with the VH domain of the first effector moiety followed by the CH1 domain of a human IgG1, which is fused to a Fc region (e.g., CH2-CH3) of human IgG1. In some embodiments, at the C-terminus of the Fc region is fused to a linker as provided herein, such as but not limited to, GGGGS (SEQ ID NO: 4), GGGGSGGGGS (SEQ ID NO: 5), GGGGSGGGGSGGGGS (SEQ ID NO: 6), GGGGSGGGGSGGGGSGGGGS (SEQ ID NO: 7), GGGGA (SEQ ID NO: 8), GGGGAGGGGA (SEQ ID NO: 9), GGGGAGGGGAGGGGA (SEQ ID NO: 10), or GGGGAGGGGAGGGGAGGGGA (SEQ ID NO: 11), GGGSE (SEQ ID NO: 300), GGGSEGGGSE (SEQ ID NO: 301), GGGSEGGGSEGGGSE (SEQ ID NO: 1), or GGGSEGGGSEGGGSEGGGSE (SEQ ID NO: 302). The linker can then be fused to the second effector moiety, such as an scFv antibody. In some embodiments, the first and second effector moiety is a PD-1 antibody. In some embodiments, the PD-1 antibody is selected from PD-1 Antibody Table 4. In some embodiments, the PD-1 antibody is selected from PD-1 Antibody Table 5. In some embodiments, the PD-1 antibody is selected from PD-1 Antibody Table 4 and PD-1 Antibody Table 5. The polypeptides can homodimerize through the heavy chain homodimerization, which results in a therapeutic compound having two effector moiety sets, such as two anti-PD-1 antibody sets.

In some embodiments, a polypeptide is provided having the following formula:
Chain 1: FAbVH-[CH1]-[CH2]-[CH3]-[Linker 1]-[scFv VK]-[Linker 2]-[scFv VH]; and/or
Chain 2: FAbVL-CK, wherein:
FAbVH=Variable heavy chain domain of a PD1 antibody as provided herein;
scFv VK=Variable light domain of a PD1 antibody as provided herein;
scFv VH=Variable heavy chain domain of a PD1 antibody as provided herein;
FAbVL=Variable light domain of a PD1 antibody as provided herein;
CH1=Constant heavy domain 1 of human immunoglobulin, such as IgG1, such as, for example, provided herein;
CH2=Constant heavy domain 2 of human immunoglobulin, such as IgG1, such as, for example, provided herein;
CH3=Constant heavy domain 3 of human immunoglobulin, such as IgG1, such as, for example, provided herein;
CK=Constant domain of kappa light chain, such as provided herein or other constant domain of a light chain that can be substituted for the constant domain of the kappa light chain;
Linker 1 is a glycine/serine, glycine/glutamic acid/serine, alanine/glutamic acid/lysine, or glycine/alanine linker, or other linkers as provided for herein (e.g. (GGGSE)n, wherein n is 1-4);
Linker 2 is a glycine/serine, glycine/glutamic acid/serine, alanine/glutamic acid/lysine, or glycine/alanine linker, or other linkers as provided for herein (e.g. (GGGSE)n, wherein n is 1-4).

In some embodiments, Chain 1 has the following formula FAbVH-[CH1]-[CH2]-[CH3]-[Linker 1]-[scFv VH]-[Linker 2]-[scFv VK]; wherein:
AbVH=Variable heavy chain domain of a PD1 antibody as provided herein;
scFv VK=Variable light domain of a PD1 antibody as provided herein;
scFv VH=Variable heavy chain domain of a PD1 antibody as provided herein;
FAbVL=Variable light domain of a PD1 antibody as provided herein;
CH1=Constant heavy domain 1 of human immunoglobulin, such as IgG1, such as, for example, provided herein;
CH2=Constant heavy domain 2 of human immunoglobulin, such as IgG1, such as, for example, provided herein;
CH3=Constant heavy domain 3 of human immunoglobulin, such as IgG1, such as, for example, provided herein;
CK=Constant domain of kappa light chain, such as provided herein or other constant domain of a light chain that can be substituted for the constant domain of the kappa light chain;
Linker 1 is a glycine/serine, glycine/glutamic acid/serine, alanine/glutamic acid/lysine, or glycine/alanine linker, or other linkers as provided for herein (e.g. (GGGSE)n, wherein n is 1-4);
Linker 2 is a glycine/serine, glycine/glutamic acid/serine, alanine/glutamic acid/lysine, or glycine/alanine linker, or other linkers as provided for herein (e.g. (GGGSE)n, wherein n is 1-4).

In some embodiments, a polypeptide is provided having the following formula:
Chain 1: [scFv VK]-[Linker 2]-[scFv VH]-[Linker 1]-[FAbVH]-[CH1]-[CH2]-[CH3] and/or
Chain 2: FAbVL-CK, wherein:
FAbVH=Variable heavy chain domain of a PD1 antibody as provided herein;
scFv VK=Variable light domain of a PD1 antibody as provided herein;
scFv VH=Variable heavy chain domain of a PD1 antibody as provided herein;
FAbVL=Variable light domain of a PD1 antibody as provided herein;
CH1=Constant heavy domain 1 of human immunoglobulin, such as IgG1, such as, for example, provided herein;
CH2=Constant heavy domain 2 of human immunoglobulin, such as IgG1, such as, for example, provided herein;
CH3=Constant heavy domain 3 of human immunoglobulin, such as IgG1, such as, for example, provided herein;
CK=Constant domain of kappa light chain, such as provided herein or other constant domain of a light chain that can be substituted for the constant domain of the kappa light chain;
Linker 1 is a glycine/serine, glycine/glutamic acid/serine, alanine/glutamic acid/lysine, or glycine/alanine linker, or other linkers as provided for herein (e.g. (GGGSE)n, wherein n is 1-4);
Linker 2 is a glycine/serine, glycine/glutamic acid/serine, alanine/glutamic acid/lysine, or glycine/alanine linker, or other linkers as provided for herein (e.g. (GGGSE)n, wherein n is 1-4.

In some embodiments, a polypeptide is provided having the following formula:
Chain 1: [scFv VH]-[Linker 2]-[scFv VK]-[Linker 1]-[FAbVH]-[CH1]-[CH2]-[CH3] and/or
Chain 2: FAbVL-CK, wherein:
FAbVH=Variable heavy chain domain of a PD1 antibody as provided herein;
scFv VK=Variable light domain of a PD1 antibody as provided herein;
scFv VH=Variable heavy chain domain of a PD1 antibody as provided herein;
FAbVL=Variable light domain of a PD1 antibody as provided herein;
CH1=Constant heavy domain 1 of human immunoglobulin, such as IgG1, such as, for example, provided herein;
CH2=Constant heavy domain 2 of human immunoglobulin, such as IgG1, such as, for example, provided herein;
CH3=Constant heavy domain 3 of human immunoglobulin, such as IgG1, such as, for example, provided herein;
CK=Constant domain of kappa light chain, such as provided herein or other constant domain of a light chain that can be substituted for the constant domain of the kappa light chain;
Linker 1 is a glycine/serine, glycine/glutamic acid/serine, alanine/glutamic acid/lysine, or glycine/alanine linker, or other linkers as provided for herein (e.g. (GGGSE)n, wherein n is 1-4);
Linker 2 is a glycine/serine, glycine/glutamic acid/serine, alanine/glutamic acid/lysine, or glycine/alanine linker, or other linkers as provided for herein (e.g. (GGGSE)n, wherein n is 1-4).

In some embodiments, the polypeptide comprises a plurality of chain 1 and a plurality of chain 2. In some embodiments, the polypeptide comprises two polypeptides of chain 1 and 2 polypeptides of chain 2. In some embodiments, the plurality (e.g., two) of polypeptides of chain 1 are linked to one another. In some embodiments, the plurality (e.g., two) of polypeptides of chain 1 are linked to one another through a disulfide bond. In some embodiments, the disulfide bond linking the plurality of chain 1 polypeptides to one another is through the [CH1]-[CH2]-[CH3] domain of the polypeptide. In some embodiments, the disulfide bond linking the plurality of chain 1 polypeptides to one another is through the hinge region present between the [CH1]-[CH2] domains of the polypeptide.

Accordingly, in some embodiments, the polypeptide can comprise four binding domains that are provided for in 4 polypeptide chains, wherein the first binding domain is formed by the FAbVH and FAbVL of the first chain 1 and first chain 2, a second binding domain is formed by the FAbVH and FAbVL of the second chain 1 and second chain 2, the third binding domain is formed by the [scFv VH/VK]-[Linker 2]-[scFv VK/VH] of the first chain 1, and the fourth binding domain is formed by the [scFv VH/VK]-[Linker 2]-[scFv VK/VH] of the second chain 1 to create a polypeptide comprising four (4) binding domains that bind to PD-1. In some embodiments, each of the binding domains act as a PD-1 agonist. In some embodiments, the 4 polypeptide binding domains comprise a sequence or antibody sequence as provided for herein. The scFV and FAb sequences (e.g. domains) are, for example, as provided for herein.

In some embodiments, the FAbVH, FAbVL, scFv VH, and scFv VH comprises a heavy chain or light chain sequence as provided for herein. In some embodiments, the FAbVH, FAbVL, scFv VH, and scFv VK comprises a CDR1, CDR2, CDR3, LCDR1, LCDR2, and a LCDR3 as provided for herein.

In some embodiments, the binding domain formed by the FAbVH and the FAbVL binds to a different epitope on PD-1 as compared to the binding domain formed by scFv VK and scFv VH.

In some embodiments, the binding domain formed by the FAbVH and the FAbVL binds to PD-1 with a higher affinity as compared to the binding domain formed by scFv VK and scFv VH.

In some embodiments, the binding domain formed by the FAbVH and the FAbVL binds to PD-1 with a higher affinity as compared to the binding domain formed by scFv VK and scFv VH.

In some embodiments, the scFv VK and scFv VH are linked by a disulfide bond. A non-limiting example of this embodiment is illustrated, for example, in FIGS. 3 (70) and (75).

In some embodiments, the PD-1 antibody is selected from the following table:

PD-1 Antibody Table 4

| Clone | | | | | | | | |
|---|---|---|---|---|---|---|---|---|
| (scFv) | VH Seq | VK Seq | CDR1 | CDR2 | CDR3 | LCDR1 | LCDR2 | LCDR3 |
| PD1AB1 | QVQLVQSGAE | DIQMTQS | GSFTGYY | GWINPN | CARDT | QASHDID | SSLQS | QQANSLPL |
| | VKKPGASVKV | PSSLSAS | MH (SEQ | DGAIHY | VTGDF | KYLN | (SEQ | T (SEQ ID |
| | SCKASGGSFT | VGDRVTI | ID NO: | A (SEQ | DYW | (SEQ ID | ID | NO: 20) |
| | GYYMHWVRQA | TCQASHD | 15) | ID NO: | (SEQ ID | NO: 18) | NO: | |
| | PGQGLEWMGW | IDKYLNW | | 16) | NO: | | 19) | |
| | INPNDGAIHY | YQQKPGK | | | 17) | | | |
| | AQNFQGRVTM | APKLLIY | | | | | | |
| | TRDTSTSTVY | AASSLQS | | | | | | |
| | MELSSLRSED | GVPSRFS | | | | | | |
| | TAVYYCARDT | GSGSGTD | | | | | | |
| | VTGDFDYWGQ | FTLTISS | | | | | | |
| | GTLVTVSS | LQPEDFA | | | | | | |
| | (SEQ ID | TYYCQQA | | | | | | |
| | NO: 13) | NSLPLTF | | | | | | |
| | | GGGTKVE | | | | | | |
| | | IK (SEQ | | | | | | |
| | | ID NO: | | | | | | |
| | | 14) | | | | | | |
| PD1AB2 | QVQLVQSGAE | DIQMTQS | GTFSRYA | GWINPN | CAKQG | RASQSIS | STLES | QQSYSTPF |
| | VKKPGASVKV | PSSLSAS | VS (SEQ | SGGTSY | DYGGG | SWLA | (SEQ | T (SEQ ID |
| | SCKASGGTFS | VGDRVTI | ID NO: | A (SEQ | YYFDY | (SEQ ID | ID | NO: 28) |
| | RYAVSWVRQA | TCRASQS | 23) | ID NO: | W (SEQ | NO: 26) | NO: | |
| | PGQGLEWMGW | ISSWLAW | | 24) | ID | | 27) | |
| | INPNSGGTSY | YQQKPGK | | | NO: | | | |
| | AQRFQGRVTM | APKLLIY | | | 25) | | | |
| | TRDTSTSTVY | KTSTLES | | | | | | |
| | MELSSLRSED | GVPSRFS | | | | | | |
| | TAVYYCAKQG | GSGSGTD | | | | | | |
| | DYGGGYYFDY | FTLTISS | | | | | | |
| | WGQGTLVTVS | LQPEDFA | | | | | | |
| | S (SEQ ID | TYYCQQS | | | | | | |
| | NO: 21) | YSTPFTF | | | | | | |
| | | GQGTKVE | | | | | | |
| | | IK (SEQ | | | | | | |
| | | ID NO: | | | | | | |
| | | 22) | | | | | | |
| PD1AB3 | QVQLVQSGAE | DIQMTQS | GTFSSYA | GWMNPN | CARVG | RASQSIN | SSLQS | QQSYSTPF |
| | VKKPGASVKV | PSSLSAS | IS (SEQ | SGNTGY | YSYGY | NWLA | (SEQ | T (SEQ ID |
| | SCKASGGTFS | VGDRVTI | ID NO: | A (SEQ | GMDVW | (SEQ ID | ID | NO: 28) |
| | SYAISWVRQA | TCRASQS | 31) | ID NO: | (SEQ | NO: 34) | NO: | |
| | PGQGLEWMGW | INNWLAW | | 32) | ID | | 19) | |
| | MNPNSGNTGY | YQQKPGK | | | NO: | | | |
| | AQKFQGRVTM | APKLLIY | | | 33) | | | |
| | TRDTSTSTVY | AASSLQS | | | | | | |
| | MELSSLRSED | GVPSRFS | | | | | | |
| | TAVYYCARVG | GSGSGTD | | | | | | |
| | YSYGYGMDVW | FTLTISS | | | | | | |
| | GQGTTVTVSS | LQPEDFA | | | | | | |
| | (SEQ ID | TYYCQQS | | | | | | |
| | NO: 29) | YSTPFTF | | | | | | |
| | | GPGTKVD | | | | | | |
| | | IK (SEQ | | | | | | |
| | | ID NO: | | | | | | |
| | | 30) | | | | | | |
| PD1AB4 | QVQLVQSGAE | DIQMTQS | YSFTTYY | GIINPS | CASGW | QASRDIK | SSLQS | QQSYSTPP |
| | VKKPGASVKV | PSSLSAS | MH (SEQ | GGSTSY | VYW | NYLA | (SEQ | T (SEQ ID |
| | SCKASGYSFT | VGDRVTI | ID NO: | A (SEQ | (SEQ ID | (SEQ ID | ID | NO: 41) |
| | TYYMHWVRQA | TCQASRD | 37) | ID NO: | NO: | NO: 40) | NO: | |
| | PGQGLEWMGI | IKNYLAW | | 38) | 39) | | 19) | |
| | INPSGGSTSY | YQQKPGK | | | | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ ID | LQPEDFA | | | | | | |
| | NO: 35) | TYYCQQS | | | | | | |
| | | YSTPPTF | | | | | | |
| | | GPGTKVD | | | | | | |
| | | IK (SEQ | | | | | | |
| | | ID NO: | | | | | | |
| | | 36) | | | | | | |
| PD1AB5 | EVQLLESGGG | DIQMTQS | FTFSSYA | AAIWSD | CARGL | RASQSIS | STLQS | QQSYSTPL |
| | LVQPGGSLRL | PSSLSAS | MS (SEQ | GSHQYY | GVERG | SWLA | (SEQ | T (SEQ ID |
| | SCAASGFTFS | VGDRVTI | ID NO: | A (SEQ | LDYW | (SEQ ID | ID | NO: 48) |
| | SYAMSWVRQA | TCRASQS | 44) | ID NO: | (SEQ | NO: 26) | NO: | |
| | PGKGLEWVAA | ISSWLAW | | 45) | ID | | 47) | |
| | IWSDGSHQYY | YQQKPGK | | | NO: | | | |
| | ADSVKGRFTI | APKLLIY | | | 46) | | | |
| | SRDNSKNTLY | AASTLQS | | | | | | |
| | LQMNSLRAED | GVPSRFS | | | | | | |
| | TAVYYCARGL | GSGSGTD | | | | | | |
| | GVERGLDYWG | FTLTISS | | | | | | |
| | QGTLVTVSS | LQPEDFA | | | | | | |
| | (SEQ ID NO: | TYYCQQS | | | | | | |
| | 42) | YSTPLTF | | | | | | |
| | | GQGTKVE | | | | | | |
| | | IK (SEQ | | | | | | |
| | | ID NO: | | | | | | |
| | | 43) | | | | | | |
| PD1AB6 | EVQLLESGGG | DIQMTQS | FTFSNYP | ALISDD | CARDS | RASQSIN | SNLET | QQSYSTPL |
| | LVQPGGSLRL | PSSLSAS | MH (SEQ | GTNEHY | KFANY | NYLS | (SEQ | T (SEQ ID |
| | SCAASGFTFS | VGDRVTI | ID NO: | A (SEQ | YYYYD | (SEQ ID | ID | NO: 48) |
| | NYPMHWVRQA | TCRASQS | 51) | ID NO: | MDVW | NO: 54) | NO: | |
| | PGKGLEWVAL | INNYLSW | | 52) | (SEQ | | 55) | |
| | ISDDGTNEHY | YQQKPGK | | | ID | | | |
| | ADSVKGRFTI | APKLLIY | | | NO: | | | |
| | SRDNSKNTLY | DASNLET | | | 53) | | | |
| | LQMNSLRAED | GVPSRFS | | | | | | |
| | TAVYYCARDS | GSGSGTD | | | | | | |
| | KFANYYYYYD | FTLTISS | | | | | | |
| | MDVWGQGTTV | LQPEDFA | | | | | | |
| | TVSS (SEQ | TYYCQQS | | | | | | |
| | ID NO: 49) | YSTPLTF | | | | | | |
| | | GPGTKVD | | | | | | |
| | | IK (SEQ | | | | | | |
| | | ID NO: | | | | | | |
| | | 50) | | | | | | |
| PD1AB7 | QVQLVQSGAE | DIVMTQS | YSFTGHY | GIINPN | CARGK | RSSQSIL | STROS | QQYYSIPV |
| | VKKPGASVKV | PDSLAVS | IH (SEQ | GGSTTY | FDFYG | YSSNNRD | (SEQ | T (SEQ ID |
| | SCKASGYSFT | LGERATI | ID NO: | A (SEQ | DYVTA | YLA (SEQ | ID | NO: 63) |
| | GHYIHWVRQA | NCRSSQS | 58) | ID NO: | FDIW | ID NO: | NO: | |
| | PGQGLEWMGI | ILYSSNN | | 59) | (SEQ | 61) | 62) | |
| | INPNGGSTTY | RDYLAWY | | | ID | | | |
| | AQKLQGRVTM | QQKPGQP | | | NO: | | | |
| | TRDTSTSTVY | PKLLIYW | | | 60) | | | |
| | MELSSLRSED | ASTRQSG | | | | | | |
| | TAVYYCARGK | VPDRFSG | | | | | | |
| | FDFYGDYVTA | SGSGTDF | | | | | | |
| | FDIWGQGTMV | TLTISSL | | | | | | |
| | TVSS (SEQ | QAEDVAV | | | | | | |
| | ID NO: 56) | YYCQQYY | | | | | | |
| | | SIPVTFG | | | | | | |
| | | GGTKVEI | | | | | | |
| | | K (SEQ | | | | | | |
| | | ID NO: | | | | | | |
| | | 57) | | | | | | |
| PD1AB8 | QVQLVQSGAE | DIQMTQS | YTFSNYD | GWMNPN | CARGA | RASQSIN | SSLQG | QQSYSFPY |
| | VKKPGASVKV | PSSLSAS | MN (SEQ | SGHTGS | FGLHL | NWLA | (SEQ | T (SEQ |
| | SCKASGYTFS | VGDRVTI | ID NO: | A (SEQ | GELSL | (SEQ ID | ID | ID NO: |
| | NYDMNWVRQA | TCRASQS | 66) | ID NO: | HYYGM | NO: 34) | NO: | 70) |
| | PGQGLEWMGW | INNWLAW | | 67) | DVW | | 69) | |
| | MNPNSGHTGS | YQQKPGK | | | (SEQ | | | |
| | APKFQGRVTM | APKLLIY | | | ID | | | |
| | TRDTSTSTVY | AASSLQG | | | NO: | | | |
| | MELSSLRSED | GVPSRFS | | | 68) | | | |
| | TAVYYCARGA | GSGSGTD | | | | | | |
| | FGLHLGELSL | FTLTISS | | | | | | |
| | HYYGMDVWGQ | LQPEDFA | | | | | | |
| | GTTVTVSS | TYYCQQS | | | | | | |
| | (SEQ ID | YSFPYTF | | | | | | |
| | NO: 64) | GQGTKLE | | | | | | |
| | | IK (SEQ | | | | | | |
| | | ID NO: | | | | | | |
| | | 65) | | | | | | |
| PD1AB9 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ ID |
| | SCKASGYTFT | VGDRVTI | ID NO: | A (SEQ | TW | (SEQ ID | ID | NO: 77) |
| | GYYMHWVRQA | TCRASQS | 73) | ID NO: | (SEQ ID | NO: 76) | NO: | |
| | PGQGLEWMGK | ISRWLAW | | 74) | NO: | | 19) | |
| | IVPMFDAANY | YQQKPGK | | | 75) | | | |
| | APKFQGRVTI | APKLLIY | | | | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID NO: | TYYCQQA | | | | | | |
| | 71) | NSFPVTF | | | | | | |
| | | GGGTKVE | | | | | | |
| | | IK (SEQ | | | | | | |
| | | ID NO: | | | | | | |
| | | 72) | | | | | | |
| PD1AB10 | QVQLVQSGAE | DIQMTQS | YTFTGYY | GIINPS | CAKTA | RASQSIN | SSLQS | QQGYSVPL |
| | VKKPGASVKV | PSSLSAS | MH (SEQ | GGSTSY | GYDWL | SWLA | (SEQ | S (SEQ ID |
| | SCKASGYTFT | VGDRVTI | ID NO: | A (SEQ | PSGLG | (SEQ ID | ID | NO: 82) |
| | GYYMHWVRQA | TCRASQS | 73) | ID NO: | MDVW | NO: 81) | NO: | |
| | PGQGLEWMGI | INSWLAW | | 38) | (SEQ | | 19) | |
| | INPSGGSTSY | YQQKPGK | | | ID | | | |
| | AQKFQGRVTM | APKLLIY | | | NO: | | | |
| | TRDTSTSTVY | YASSLQS | | | 80) | | | |
| | MELSSLRSED | GVPSRFS | | | | | | |
| | TAVYYCAKTA | GSGSGTD | | | | | | |
| | GYDWLPSGLG | FTLTISS | | | | | | |
| | MDVWGQGTTV | LQPEDFA | | | | | | |
| | TVSS (SEQ | TYYCQQG | | | | | | |
| | ID NO: 78) | YSVPLSF | | | | | | |
| | | GQGTKLE | | | | | | |
| | | IK (SEQ | | | | | | |
| | | ID NO: | | | | | | |
| | | 79) | | | | | | |
| PD1AB11 | QVQLVQSGAE | DIQMTQS | YTFSNYG | GGIIPI | CARWR | RASQGIS | SNLET | QQSYSTPL |
| | VKKPGSSVKV | PSSLSAS | IT (SEQ | FGSTAS | SDAFD | NWLA | (SEQ | T (SEQ ID |
| | SCKASGYTFS | VGDRVTI | ID NO: | YA (SEQ | IW | (SEQ ID | ID | NO: 48) |
| | NYGITWVRQA | TCRASQG | 85) | ID NO: | (SEQ ID | NO: 88) | NO: | |
| | PGQGLEWMGG | ISNWLAW | | 86) | NO: | | 55) | |
| | IIPIFGSTAS | YQQKPGK | | | 87) | | | |
| | YAQKFQGRVT | APKLLIY | | | | | | |
| | ITADESTSTA | DASNLET | | | | | | |
| | YMELSSLRSE | GVPSRFS | | | | | | |
| | DTAVYYCARW | GSGSGTD | | | | | | |
| | RSDAFDIWGQ | FTLTISS | | | | | | |
| | GTMVTVSS | LQPEDFA | | | | | | |
| | (SEQ ID NO: | TYYCQQS | | | | | | |
| | 83) | YSTPLTF | | | | | | |
| | | GGGTKVE | | | | | | |
| | | IK (SEQ | | | | | | |
| | | ID NO: | | | | | | |
| | | 84) | | | | | | |
| PD1AB12 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ ID |
| | SCKASGGTFS | VGDRVTI | ID NO: | A (SEQ | GMDVW | (SEQ ID | ID | NO: 28) |
| | TYAISWVRQA | TCRASQG | 91) | ID NO: | (SEQ | NO: 94) | NO: | |
| | PGQGLEWMGW | IRNDLGW | | 92) | ID | | 95) | |
| | INPNSGGTNY | YQQKPGK | | | NO: | | | |
| | AQKFQGRVTM | APKLLIY | | | 93) | | | |
| | TRDTSTSIVY | RASTLNS | | | | | | |
| | MELSSLRSED | GVPSRFS | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 89) | YSTPFTF | | | | | | |
| | | GPGTKVD | | | | | | |
| | | IK (SEQ | | | | | | |
| | | ID NO: | | | | | | |
| | | 90) | | | | | | |
| PD1AB13 | QVQLVQSGAE | DIQMTQS | GTFSTYA | GWINPN | CARVN | RASQGIR | STLNS | QQSYSTPF |
| | VKKPGASVKV | PSSLSAS | IS (SEQ | SGGTNY | YDFYY | NDLG | (SEQ | T (SEQ ID |
| | SCKASGGTFS | VGDRVTI | ID NO: | A (SEQ | GMDVW | (SEQ ID | ID | NO: 28) |
| | TYAISWVRQA | TCRASQG | 91) | ID NO: | (SEQ | NO: 94) | NO: | |
| | PGQGLEWMGW | IRNDLGW | | 92) | ID | | 95) | |
| | INPNSGGTNY | YQQKPGK | | | NO: | | | |
| | AQKFQGRVTM | APKLLIY | | | 93) | | | |
| | TRDTSTSTVY | RASTLNS | | | | | | |
| | MELSSLRSED | GVPSRFS | | | | | | |
| | TAVYYCARVN | GSGSGTD | | | | | | |
| | YDFYYGMDVW | FTLTISS | | | | | | |
| | GQGTTVTVSS | LQPEDFA | | | | | | |
| | (SEQ ID | TYYCQQS | | | | | | |
| | NO: 96) | YSTPFTF | | | | | | |
| | | GPGTKVD | | | | | | |
| | | IK (SEQ | | | | | | |
| | | ID NO: | | | | | | |
| | | 90) | | | | | | |
| PD1AB14 | EVQLLESGGG | DIQMTQS | FSFSSYD | SGISGS | CASPY | RASQDIA | SSVQT | QQSYTTPY |
| | LVQPGGSLRL | PSSLSAS | MS (SEQ | GSSTYY | GMGYM | NYLA | (SEQ | T (SEQ ID |
| | SCAASGFSFS | VGDRVTI | ID NO: | A (SEQ | DVW | (SEQ ID | ID | NO: 104) |
| | SYDMSWVRQA | TCRASQD | 99) | ID NO: | (SEQ ID | NO: | NO: | |
| | PGKGLEWVSG | IANYLAW | | 100) | NO: | 102) | 103) | |
| | ISGSGSSTYY | YQQKPGK | | | 101) | | | |
| | ADSVKGRFTI | APKLLIY | | | | | | |
| | SRDNSKNTLY | GASSVQT | | | | | | |
| | LQMNSLRAED | GVPSRFS | | | | | | |
| | TAVYYCASPY | GSGSGTD | | | | | | |
| | GMGYMDVWGK | FTLTISS | | | | | | |
| | GTTVTVSS | LQPEDFA | | | | | | |
| | (SEQ ID NO: | TYYCQQS | | | | | | |
| | 97) | YTTPYTF | | | | | | |
| | | GQGTRLE | | | | | | |
| | | IK (SEQ | | | | | | |
| | | ID NO: | | | | | | |
| | | 98) | | | | | | |
| PD1AB15 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ ID |
| | SCKASGGSFN | VGDRVTI | ID NO: | A (SEQ | YMDVW | (SEQ ID | ID | NO: 28) |
| | NYAISWVRQA | TCQASQD | 107) | ID NO: | (SEQ | NO: | NO: | |
| | PGQGLEWMGW | ISRYLNW | | 108) | ID | 110) | 111) | |
| | INPNTGGTSY | YQQKPGK | | | NO: | | | |
| | AQKFQGRVTM | APKLLIY | | | 109) | | | |
| | TRDTSTSTVY | AASNLQS | | | | | | |
| | MELSSLRSED | GVPSRFS | | | | | | |
| | TAVYYCARVS | GSGSGTD | | | | | | |
| | YGVGYYMDVW | FTLTISS | | | | | | |
| | GKGTTVTVSS | LQPEDFA | | | | | | |
| | (SEQ ID | TYYCQQS | | | | | | |
| | NO: 105) | YSTPFTF | | | | | | |
| | | GPGTKVD | | | | | | |
| | | IK (SEQ | | | | | | |
| | | ID NO: | | | | | | |
| | | 106) | | | | | | |
| PD1AB16 | QVQLVQSGAE | DIQMTQS | GSFNNYA | GWINPN | CARVS | QASQDIS | SNLQS | QQSYSTPF |
| | VKKPGASVKV | PSSLSAS | IS (SEQ | TGGTSY | YGVGY | RYLN | (SEQ | T (SEQ ID |
| | SCKASGGSFN | VGDRVTI | ID NO: | A (SEQ | YMDVW | (SEQ ID | ID | NO: 28) |
| | NYAISWVRQA | TCQASQD | 107) | ID NO: | (SEQ | NO: | NO: | |
| | PGQGLEWMGW | ISRYLNW | | 108) | ID | 110) | 111) | |
| | INPNTGGTSY | YQQKPGK | | | NO: | | | |
| | AQKFQGRVTM | APKLLIY | | | 109) | | | |
| | TRDTSTSTVY | AASNLQS | | | | | | |
| | MELSSLRSED | GVPSRFS | | | | | | |
| | TAVYYCARVS | GSGSGTD | | | | | | |
| | YGVGYYMDVW | STLTISS | | | | | | |
| | GKGTTVTVSS | LQPEDFA | | | | | | |
| | (SEQ ID | TYYCQQS | | | | | | |
| | NO: 105) | YSTPFTF | | | | | | |
| | | GPGTKVD | | | | | | |
| | | IK (SEQ | | | | | | |
| | | ID NO: | | | | | | |
| | | 112) | | | | | | |
| PD1AB17 | QVQLVQSGAE | DIQMTQS | YTFTDDY | GWMNTN | CARGG | RASQGVG | SSLQS | QQAYSFPW |
| | VKKPGASVKV | PSSLSAS | IH (SEQ | SGNTGY | SYSSG | NALG | (SEQ | T (SEQ ID |
| | SCKASGYTFT | VGDRVTI | ID NO: | A (SEQ | WYGRL | (SEQ ID | ID | NO: 119) |
| | DDYIHWVRQA | TCRASQG | 115) | ID NO: | DYYYG | NO: | NO: | |
| | PGQGLEWMGW | VGNALGW | | 116) | MDVW | 118) | 19) | |
| | MNTNSGNTGY | YQQKPGK | | | (SEQ | | | |
| | AQKFQGRVTM | APKLLIY | | | ID | | | |
| | TRDTSTSTVY | AASSLQS | | | NO: | | | |
| | MELSSLRSED | GVPSRFS | | | 117) | | | |
| | TAVYYCARGG | GSGSGTD | | | | | | |
| | SYSSGWYGRL | FTLTISS | | | | | | |
| | DYYYGMDVWG | LQPEDFA | | | | | | |
| | QGTTVTVSS | TYYCQQA | | | | | | |
| | (SEQ ID NO: | YSFPWTF | | | | | | |
| | 113) | GQGTKLE | | | | | | |
| | | IK (SEQ | | | | | | |
| | | ID NO: | | | | | | |
| | | 114) | | | | | | |
| PD1AB18 | QVQLVQSGAE | DIQMTQS | YTFTDYA | GWLNPN | CAAGL | RASQSIN | SSLES | QQSYSIPI |
| | VKKPGASVKV | PSSLSAS | MH (SEQ | SGNTGY | FIW | RWLA | (SEQ | T (SEQ ID |
| | SCKASGYTFT | VGDRVTI | ID NO: | A (SEQ | (SEQ ID | (SEQ ID | ID | NO: 127) |
| | DYAMHWVRQA | TCRASQS | 122) | ID NO: | NO: | NO: | NO: | |
| | PGQGLEWMGW | INRWLAW | | 123) | 124) | 125) | 126 | |
| | LNPNSGNTGY | YQQKPGK | | | | | | |
| | APKFQGRVTM | APKLLIY | | | | | | |
| | TRDTSTSTVY | DASSLES | | | | | | |
| | MELSSLRSED | GVPSRFS | | | | | | |
| | TAVYYCAAGL | GSGSGTD | | | | | | |
| | FIWGQGTMVT | FTLTISS | | | | | | |
| | VSS (SEQ ID | LQPEDFA | | | | | | |
| | NO: 120) | TYYCQQS | | | | | | |
| | | YSIPITF | | | | | | |
| | | GQGTRLE | | | | | | |
| | | IK (SEQ | | | | | | |
| | | ID NO: | | | | | | |
| | | 121) | | | | | | |
| PD1AB19 | QVQLVQSGAE | DIVMTQS | GTFSSYA | GGIIPG | CTTEY | RSSQSLL | SNRAP | MQALQTPL |
| | VKKPGSSVKV | PLSLPVT | IS (SEQ | FGSPNY | CSSTS | HSNGYNY | (SEQ | T (SEQ ID |
| | SCKASGGTFS | PGEPASI | ID NO: | A (SEQ | CSDYW | LD (SEQ | ID | NO: 134) |
| | SYAISWVRQA | SCRSSQS | 31) | ID NO: | (SEQ | ID NO: | NO: | |
| | PGQGLEWMGG | LLHSNGY | | 130) | ID | 132) | 133) | |
| | IIPGFGSPNY | NYLDWYL | | | NO: | | | |
| | APNFQGRVTI | QKPGQSP | | | 131) | | | |
| | TADESTSTAY | QLLIYQG | | | | | | |
| | MELSSLRSED | SNRAPGV | | | | | | |
| | TAVYYCTTEY | PDRFSGS | | | | | | |
| | CSSTSCSDYW | GSGTDFT | | | | | | |
| | GQGTLVTVSS | LKISRVE | | | | | | |
| | (SEQ ID | AEDVGVY | | | | | | |
| | NO: 128) | YCMQALQ | | | | | | |
| | | TPLTFGQ | | | | | | |
| | | GTKVEIK | | | | | | |
| | | (SEQ ID | | | | | | |
| | | NO: | | | | | | |
| | | 129) | | | | | | |
| PD1AB20 | QVQLVQSGAE | DIQMTQS | YTFSDHY | GTINPS | CAADN | RASQSIS | STLQS | QQSHSLPL |
| | VKKPGASVKV | PSSLSAS | MH (SEQ | GGRTSY | GHASG | NWVA | (SEQ | T (SEQ ID |
| | SCKASGYTFS | VGDRVTI | ID NO: | A (SEQ | WLYYY | (SEQ ID | ID | NO: 141) |
| | DHYMHWVRQA | TCRASOS | 137) | ID NO: | GMDVW | NO: | NO: | |
| | PGQGLEWMGT | ISNWVAW | | 138) | (SEQ | 140) | 47) | |
| | INPSGGRTSY | YQQKPGK | | | ID | | | |
| | AQKFQGRVTM | APKLLIY | | | NO: | | | |
| | TRDTSTSTVY | RASTLQS | | | 139) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 135) | GPGTKVD | | | | | | |
| | | IK (SEQ | | | | | | |
| | | ID NO: | | | | | | |
| | | 136) | | | | | | |
| PD1AB21 | EVQLLESGGG | DIQMTQS | FTFSSYA | SGISGG | CASEY | RASQSIS | SSLQS | QQYRNFPY |
| | LVQPGGSLRL | PSSLSAS | MS (SEQ | GGTTYY | YGMDV | GWLA | (SEQ | T (SEQ ID |
| | SCAASGFTFS | VGDRVTI | ID NO: | A (SEQ | W (SEQ | (SEQ ID | ID | NO: 147) |
| | SYAMSWVRQA | TCRASQS | 44) | ID NO: | ID | NO: | NO: | |
| | PGKGLEWVSG | ISGWLAW | | 144) | NO: | 146) | 19) | |
| | ISGGGGTTYY | YQQKPGK | | | 145) | | | |
| | ADSVKGRFTI | APKLLIY | | | | | | |
| | SRDNSKNTLY | AASSLQS | | | | | | |
| | LQMNSLRAED | GVPSRFS | | | | | | |
| | TAVYYCASEY | GSGSGTD | | | | | | |
| | YGMDVWGQGT | FTLTISS | | | | | | |
| | TVTVSS (SEQ | LQPEDFA | | | | | | |
| | ID NO: | TYYCQQY | | | | | | |
| | 142) | RNFPYTF | | | | | | |
| | | GQGTKLE | | | | | | |
| | | IK (SEQ | | | | | | |
| | | ID NO: | | | | | | |
| | | 143) | | | | | | |
| PD1AB22 | QVQLVQSGAE | EIVMTQS | YTFSGYY | GVINPS | CAEGF | RASQGVG | STRAT | QQYYTTPI |
| | VKKPGASVKV | PATLSVS | MH (SEQ | GGSTSY | DYW | RSLA | (SEQ | T (SEQ ID |
| | SCKASGYTFS | PGERATL | ID NO: | A (SEQ | (SEQ ID | (SEQ ID | ID | NO: 155) |
| | GYYMHWVRQA | SCRASQG | 150) | ID NO: | NO: | NO: | NO: | |
| | PGQGLEWMGV | VGRSLAW | | 151) | 152) | 153) | 154) | |
| | INPSGGSTSY | YQQKPGQ | | | | | | |
| | AQKFQGRVTM | APRLLIY | | | | | | |
| | TRDTSTSTVY | GASTRAT | | | | | | |
| | MELSSLRSED | GIPARFS | | | | | | |
| | TAVYYCAEGF | GSGSGTE | | | | | | |
| | DYWGQGTLVT | FTLTISS | | | | | | |
| | VSS (SEQ ID | LQSEDFA | | | | | | |
| | NO: 148) | VYYCQQY | | | | | | |
| | | YTTPITF | | | | | | |
| | | GOGTRLE | | | | | | |
| | | IK (SEQ | | | | | | |
| | | ID NO: | | | | | | |
| | | 149) | | | | | | |
| PD1AB23 | QVQLVQSGAE | DIQMTQS | GTFSNYA | GWMNPN | CARVN | QASQDIS | STLKS | QQADNLPF |
| | VKKPGASVKV | PSSLSAS | IS (SEQ | SGNTGY | YYYYY | NYLN | (SEQ | T (SEQ ID |
| | SCKASGGTFS | VGDRVTI | ID NO: | A (SEQ | GMDVW | (SEQ ID | ID | NO: 162) |
| | NYAISWVRQA | TCQASQD | 158) | ID NO: | (SEQ | NO: | NO: | |
| | PGQGLEWMGW | ISNYLNW | | 32) | ID | 160) | 161) | |
| | MNPNSGNTGY | YQQKPGK | | | NO: | | | |
| | AQKFQGRVTM | APKLLIY | | | 159) | | | |
| | TRDTSTSTVY | KASTLKS | | | | | | |
| | MELSSLRSED | GVPSRFS | | | | | | |
| | TAVYYCARVN | GSGSGTD | | | | | | |
| | YYYYYGMDVW | FTLTISS | | | | | | |
| | GQGTTVTVSS | LQPEDFA | | | | | | |
| | (SEQ ID | TYYCQQA | | | | | | |
| | NO: 156) | DNLPFTF | | | | | | |
| | | GPGTKVD | | | | | | |
| | | IK (SEQ | | | | | | |
| | | ID NO: | | | | | | |
| | | 157) | | | | | | |
| PD1AB24 | QVQLVQSGAE | DIQMTQS | YTFTNYY | GIINPS | CARDW | QASRDIS | SSLQS | QQANSFPP |
| | VKKPGASVKV | PSSLSAS | MH (SEQ | GGSTSY | GWDYY | NYLN | (SEQ | T (SEQ ID |
| | SCKASGYTFT | VGDRVTI | ID NO: | A (SEQ | YYGMD | (SEQ ID | ID | NO: 168) |
| | NYYMHWVRQA | TCQASRD | 165) | ID NO: | VW | NO: | NO: | |
| | PGQGLEWMGI | ISNYLNW | | 38) | (SEQ ID | 167) | 19) | |
| | INPSGGSTSY | YQQKPGK | | | NO: | | | |
| | AQRFQGRVTM | APKLLIY | | | 166) | | | |
| | TRDTSTSTVY | AASSLQS | | | | | | |
| | MELSSLRSED | GVPSRFS | | | | | | |
| | TAVYYCARDW | GSGSGTD | | | | | | |
| | GWDYYYYGMD | FTLTISS | | | | | | |
| | VWGQGTTVTV | LQPEDFA | | | | | | |
| | SS (SEQ ID | TYYCQQA | | | | | | |
| | NO: 163) | NSFPPTF | | | | | | |
| | | GQGTKLE | | | | | | |
| | | IK (SEQ | | | | | | |
| | | ID NO: | | | | | | |
| | | 164) | | | | | | |
| PD1AB25 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T(SEQ ID |
| | SCAASGFTFS | LGERATI | ID NO: | A (SEQ | LDYW | YLA (SEQ | ID | NO: 176) |
| | NSDMSWVRQA | NCKSSQS | 171) | ID NO: | (SEQ | ID NO: | NO: | |
| | PGKGLEWVSG | VLYSPNN | | 172) | ID | 174) | 175) | |
| | ITISGGSTYY | KNYLAWY | | | NO: | | | |
| | ADSVRGRFTI | QQKPGQP | | | 173) | | | |
| | SRDNSKNTLY | PKLLIYW | | | | | | |
| | LQMSSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID NO: | QAEDVAV | | | | | | |
| | 169) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 170) | | | | | | |
| PD1AB26 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ ID |
| | SCKASGYTFT | VGDRVTI | ID NO: | A (SEQ | TW | (SEQ ID | ID | NO: 77) |
| | GYYMHWVRQA | TCRASQS | 73) | ID NO: | (SEQ ID | NO: 76) | NO: | |
| | PGQGLEWMGK | ISRWLAW | | 74) | NO: | | 19) | |
| | IVPMFDAANY | YQQKPGK | | | 75) | | | |
| | APKFQGRVTI | APKLLIY | | | | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTLTISS | | | | | | |
| | TLVTVSS | LQPEDFA | | | | | | |
| | (SEQ ID NO: | TYYCQQA | | | | | | |
| | 71) | NSFPVTF | | | | | | |
| | | GGGTKVD | | | | | | |
| | | IK (SEQ | | | | | | |
| | | ID NO: | | | | | | |
| | | 177) | | | | | | |
| PD1AB27 | QVQLVQSGAE | DIQMTQS | YTFTGYY | GKIVPM | CARGP | RASQSIS | SSLQS | QQANSFPV |
| | VKKPGSSVKV | PSSLSAS | MH (SEQ | FDAANY | KWELD | RWLA | (SEQ | T (SEQ ID |
| | SCKASGYTFT | VGDRVTI | ID NO: | A (SEQ | TW | (SEQ ID | ID | NO: 77) |
| | GYYMHWVRQA | TCRASQS | 73) | ID NO: | (SEQ ID | NO: 76) | NO: | |
| | PGQGLEWMGK | ISRWLAW | | 74) | NO: | | 19) | |
| | IVPMFDAANY | YQQKPGK | | | 75) | | | |
| | APKFQGRVTI | APKLLIY | | | | | | |
| | TADESTSTAY | GASSLQS | | | | | | |
| | MELSSLRSED | GVPSRFS | | | | | | |
| | TAVYYCARGP | GSGSGTD | | | | | | |
| | KWELDTWGQG | FTFTISS | | | | | | |
| | TLVTVSS | LQPEDFA | | | | | | |
| | (SEQ ID NO: | TYYCQQA | | | | | | |
| | 71) | NSFPVTF | | | | | | |
| | | GGGTKVD | | | | | | |
| | | IK (SEQ | | | | | | |
| | | ID NO: | | | | | | |
| | | 178) | | | | | | |
| PD1AB28 | QVQLVQSGAE | DIQMTQS | GDFSNYF | GWINPH | CARGG | RASQSIS | STLQS | QQSYSTPF |
| | VKKPGSSVKV | PSSLSAS | VS (SEQ | NGDTMY | YSYGY | TWLA | (SEQ | T (SEQ ID |
| | SCKASGGDFS | VGDRVTI | ID NO: | A (SEQ | TFDIW | (SEQ ID | ID | NO: 28) |
| | NYFVSWVRQA | TCRASQS | 181) | ID NO: | (SEQ | NO: | NO: | |
| | PGQGLEWMGW | ISTWLAW | | 182) | ID | 184) | 47) | |
| | INPHNGDTMY | YQQKPGK | | | NO: | | | |
| | AQKFQGRVTI | APKLLIY | | | 183) | | | |
| | TADESTSTAY | AASTLQS | | | | | | |
| | MELSSLRSED | GVPSRFS | | | | | | |
| | TAVYYCARGG | GSGSGTD | | | | | | |
| | YSYGYTFDIW | FTLTISS | | | | | | |
| | GQGTMVTVSS | LQPEDFA | | | | | | |
| | (SEQ ID | TYYCQQS | | | | | | |
| | NO: 179) | YSTPFTF | | | | | | |
| | | GGGTKVE | | | | | | |
| | | IK (SEQ | | | | | | |
| | | ID NO: | | | | | | |
| | | 180) | | | | | | |
| PD1AB29 | EVQLLESGGG | DIVMTQS | FTFSNSD | SGITIS | CARGR | KSSQSVL | STRES | QQYYITPP |
| | LVQPGGSLRL | PDSLAVS | MS (SEQ | GGSTYY | GGSGW | YSPNNKN | (SEQ | T (SEQ ID |
| | SCAASGFTFS | LGERATI | ID NO: | A (SEQ | LDYW | YLA (SEQ | ID | NO: 187) |
| | NSDMSWVRQA | NCKSSQS | 171) | ID NO: | (SEQ | ID NO: | NO: | |
| | PGKGLEWVSG | VLYSPNN | | 172) | ID | 174) | 175) | |
| | ITISGGSTYY | KNYLAWY | | | NO: | | | |
| | ADSVKGRFTI | QQKPGQP | | | 173) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID NO: | QAEDVAV | | | | | | |
| | 185) | YYCQQYY | | | | | | |
| | | ITPPTFG | | | | | | |
| | | QGTRLEI | | | | | | |
| | | K (SEQ | | | | | | |
| | | ID NO: | | | | | | |
| | | 186) | | | | | | |
| PD1AB30 | EVQLLESGGG | DIVMTQS | FTFSNSD | SGITIS | CARGR | KSSQSVL | STRES | QQYYTTPP |
| | LVQPGGSLRL | PDSLAVS | MS (SEQ | GGSTYY | GGSGW | YSPNNKN | (SEQ | T (SEQ ID |
| | SCAASGFTFS | LGERATI | ID NO: | A (SEQ | LDYW | YLA (SEQ | ID | NO: 176) |
| | NSDMSWVRQA | NCKSSQS | 171) | ID NO: | (SEQ | ID NO: | NO: | |
| | PGKGLEWVSG | VLYSPNN | | 172) | ID | 174) | 175) | |
| | ITISGGSTYY | KNYLAWY | | | NO: | | | |
| | ADSVKGRFTI | QQKPGQP | | | 173) | | | |
| | SRDNSKNTLY | PKLLIYW | | | | | | |
| | LQMNSLRAED | ASTRESG | | | | | | |
| | TAVYYCARGR | VPDRFSG | | | | | | |
| | GGSGWLDYWG | SGSGTDF | | | | | | |
| | QGTLVTVSS | TLTISSL | | | | | | |
| | (SEQ ID NO: | QAEDVAV | | | | | | |
| | 185) | YYCQQYY | | | | | | |
| | | TTPPTFG | | | | | | |
| | | QGTRLEI | | | | | | |
| | | K (SEQ | | | | | | |
| | | ID NO: | | | | | | |
| | | 170) | | | | | | |
| PD1AB31 | QVQLVQSGAE | EIVMTQS | HTFTDYY | GIINPS | CASGW | RASQSVS | SSRAT | QQYTTSPI |
| | VKKPGSSVKV | PATLSVS | MH (SEQ | GGSTSY | TDW | SYLA | (SEQ | T (SEQ ID |
| | SCKASGHTFT | PGERATL | ID NO: | A (SEQ | (SEQ ID | (SEQ ID | ID | NO: 194) |
| | DYYMHWVRQA | SCRASQS | 190) | ID NO: | NO: | NO: | NO: | |
| | PGQGLEWMGI | VSSYLAW | | 38) | 191) | 192) | 193) | |
| | INPSGGSTSY | YQQKPGQ | | | | | | |
| | AQKFQGRVTI | APRLLIY | | | | | | |
| | TADESTSTAY | GTSSRAT | | | | | | |
| | MELSSLRSED | GIPARFS | | | | | | |
| | TAVYYCASGW | GSGSGTE | | | | | | |
| | TDWGQGTLVT | FTLTISS | | | | | | |
| | VSS (SEQ ID | LQSEDFA | | | | | | |
| | NO: 188) | VYYCQQY | | | | | | |
| | | TTSPITF | | | | | | |
| | | GQGTRLE | | | | | | |
| | | IKR (SEQ | | | | | | |
| | | ID NO: | | | | | | |
| | | 189) | | | | | | |
| PD1AB32 | QVQLVQSGAE | DIQMTQS | YTFTDYY | GGIFPV | CARDH | QASQDIS | KDLHP | QESFSTLT |
| | VKKPGASVKV | PSSLSAS | MH (SEQ | FGSSTY | GSGLD | NYLN | (SEQ | (SEQ ID |
| | SCKASGYTFT | VGDRVTI | ID NO: | A (SEQ | VW | (SEQ ID | ID | NO: 201) |
| | DYYMHWVRQA | TCQASQD | 197) | ID NO: | (SEQ ID | NO: | NO: | |
| | PGQGLEWMGG | ISNYLNW | | 198) | NO: | 160) | 200) | |
| | IFPVFGSSTY | YQQKPGK | | | 199) | | | |
| | AQKFQGRVTM | APKLLIY | | | | | | |
| | TRDTSTSTVY | DAKDLHP | | | | | | |
| | MELSSLRSED | GVPSRFS | | | | | | |
| | TAVYYCARDH | GSGSGTD | | | | | | |
| | GSGLDVWGQG | FTLTISS | | | | | | |
| | TTVTVSS | LQPEDFA | | | | | | |
| | (SEQ ID NO: | TYYCQES | | | | | | |
| | 195) | FSTLTFG | | | | | | |
| | | QGTKVEI | | | | | | |
| | | KR (SEQ | | | | | | |
| | | ID NO: | | | | | | |
| | | 196) | | | | | | |
| PD1AB33 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ ID |
| | SCKASGYSFT | VGDRVTI | ID NO: | A (SEQ | (SEQ ID | (SEQ ID | ID | NO: 41) |
| | TYYMHWVRQA | TCQASRD | 37) | ID NO: | NO: | NO: 40) | NO: | |
| | PGQGLEWMGI | IKNYLAW | | 203) | 39) | | 19) | |
| | IAPSGGSTSY | YQQKPGK | | | | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ ID | LQPEDFA | | | | | | |
| | NO: 202) | TYYCQQS | | | | | | |
| | | YSTPPTF | | | | | | |
| | | GPGTKVD | | | | | | |
| | | IK (SEQ | | | | | | |
| | | ID NO: | | | | | | |
| | | 36) | | | | | | |
| PD1AB34 | QVQLVQSGAE | DIQMTQS | YSFTTYY | GIIAPS | CASGW | QASRDIK | SSLQS | QQSYSTPP |
| | VKKPGASVKV | PSSLSAS | MH (SEQ | GGSTSY | VYW | NYLA | (SEQ | T (SEQ ID |
| | SCKASGYSFT | VGDRVTI | ID NO: | A (SEQ | (SEQ ID | (SEQ ID | ID | NO: 41) |
| | TYYMHWVRQA | TCQASRD | 37) | ID NO: | NO: | NO: 40) | NO: | |
| | PGQGLEWMGI | IKNYLAW | | 203) | 39) | | 19) | |
| | IGPSGGSTSY | YQQKPGK | | | | | | |
| | AQKFQGRVTM | APKLLIY | | | | | | |
| | TRDTSTSTVY | AASSLQS | | | | | | |
| | MELSSLRSED | GVPSRFS | | | | | | |
| | TAVYYCASGW | GSGSGTD | | | | | | |
| | VYWGQGTLVT | FTLTISS | | | | | | |
| | VSS (SEQ ID | LQPEDFA | | | | | | |
| | NO: 204) | TYYCQQS | | | | | | |
| | | YSTPPTF | | | | | | |
| | | GPGTKVD | | | | | | |
| | | IK (SEQ | | | | | | |
| | | ID NO: | | | | | | |
| | | 36) | | | | | | |
| PD1AB35 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ ID |
| | SCKASGYTFS | VGDRVTI | ID NO: | A (SEQ | WLYYY | (SEQ ID | ID | NO: 141) |
| | DHYMHWVRQA | TCRASQS | 137) | ID NO: | GMDVW | NO: | NO: | |
| | PGQGLEWMGT | ISNWVAW | | 206) | (SEQ | 140) | 47) | |
| | IAPSGGRTSY | YQQKPGK | | | ID | | | |
| | AQKFQGRVTM | APKLLIY | | | NO: | | | |
| | TRDTSTSTVY | RASTLQS | | | 139) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 205) | GPGTKVD | | | | | | |
| | | IK (SEQ | | | | | | |
| | | ID NO: | | | | | | |
| | | 136) | | | | | | |
| PD1AB36 | QVQLVQSGAE | DIQMTQS | YTFSDHY | GTIAPS | CAADN | RASQSIS | STLQS | QQSHSLPL |
| | VKKPGASVKV | PSSLSAS | MH (SEQ | GGRTSY | GHASG | NWVA | (SEQ | T (SEQ ID |
| | SCKASGYTFS | VGDRVTI | ID NO: | A (SEQ | WLYYY | (SEQ ID | ID | NO: 141) |
| | DHYMHWVRQA | TCRASQS | 137) | ID NO: | GMDVW | NO: | NO: | |
| | PGQGLEWMGT | ISNWVAW | | 206) | (SEQ | 140) | 47) | |
| | IGPSGGRTSY | YQQKPGK | | | ID | | | |
| | AQKFQGRVTM | APKLLIY | | | NO: | | | |
| | TRDTSTSTVY | RASTLQS | | | 139) | | | |
| | MELSSLRSED | GVPSRFS | | | | | | |
| | TAVYYCAADN | GSGSGTD | | | | | | |
| | GHASGWLYYY | FTLTISS | | | | | | |
| | GMDVWGQGTT | LQPEDFA | | | | | | |
| | VTVSS (SEQ | TYYCQQS | | | | | | |
| | ID NO: | HSLPLTF | | | | | | |
| | 207) | GPGTKVD | | | | | | |
| | | IK (SEQ | | | | | | |
| | | ID NO: | | | | | | |
| | | 136) | | | | | | |

In some embodiments, the antibody comprises a CDR set as set forth in PD-1 Antibody Table 4. In some embodiments, the antibody comprises the CDRs of Clone ID: PD1AB4, or PD1AB30 of PD-1 Antibody Table 4.

In some embodiments, FAbVH, FAbVL, scFv VH, and scFv VH comprise a CDR set as set forth in the tables referenced herein.

Although PD-1 Antibody Table 4 illustrates the heavy and light chains in what could be considered a Fab format, the heavy and light chains could be linked in a scFV format using a peptide or other type of linker to link the heavy and light chain in a single chain format.

In some embodiments, the PD-1 antibody that is in a FAb format has a lower affinity for PD-1 as compared to the PD-1 antibody that in the scFv format. In some embodiments, the PD-1 antibody that is in a FAb format has a higher affinity for PD-1 as compared to the PD-1 antibody that in the scFv format. In some embodiments, the affinity is about, or at least, 10%, 20%, 30%, 40%, 50%, 60%, 70%, 80%, 90%, 100%, 200%, 300%, 400%, or 500% higher or lower as provided for herein.

In some embodiments, the CDRs of the following clones are provided, which are based on different formats that can be used to characterize CDRs.

PD-1 Antibody Table 5

| | CDR | | | | | | |
|---|---|---|---|---|---|---|---|
| | Annotation | | | | | | |
| Clone | System | LCDR1 | LCDR2 | LCDR3 | HCDR1 | HCDR2 | HCDR3 |
| PD1AB18 | Proprietary | RASQSINRWL | SSLES | QQSYSI | YTFTD | GWLNPNS | CAAGLFIW |
| | | A (SEQ ID | (SEQ ID | PIT | YAMH | GNTGYA | (SEQ ID |
| | | NO: 125) | NO: 126) | (SEQ | (SEQ | (SEQ ID | NO: 124) |
| | | | | ID NO: | ID | NO: | |
| | | | | 127) | NO: | 123) | |
| | | | | | 122) | | |
| | Kabat | RASQSINRWL | DASSLES | QQSYSI | DYAMH | WLNPNSG | AAGLFI |
| | | A (SEQ ID | (SEQ ID | PIT | (SEQ | NTGYAPK | (SEQ ID |
| | | NO: 125) | NO: 208) | (SEQ | ID | FQG | NO: 211) |
| | | | | ID NO: | NO: | (SEQ ID | |
| | | | | 127) | 209) | NO: | |
| | | | | | | 210) | |
| | IMGT | QSINRW | DAS | QQSYSI | GYTFT | LNPNSGN | AAGLFI |
| | | (SEQ ID | | PIT | DYA | T (SEQ | (SEQ ID |
| | | NO: 212) | | (SEQ | (SEQ | ID NO: | NO: 211) |
| | | | | ID NO: | ID | 214) | |
| | | | | 127) | NO: | | |
| | | | | | 213) | | |
| | CHOTHIA | RASQSINRWL | DASSLES | QQSYSI | GYTFT | NPNSGN | LF |
| | | A (SEQ ID | (SEQ ID | PIT | DY | (SEQ ID | |
| | | NO: 125) | NO: 208) | (SEQ | (SEQ | NO: | |
| | | | | ID NO: | ID | 216) | |
| | | | | 127) | NO: | | |
| | | | | | 215) | | |
| PD1AB20 | Proprietary | RASQSISNWV | STLQS | QQSHSL | YTFSD | GTINPSG | CAADNGHA |
| | | A (SEQ ID | (SEQ ID | PLT | HYMH | GRTSYA | SGWLYYYG |
| | | NO: 140) | NO: 47) | (SEQ | (SEQ | (SEQ ID | MDVW |
| | | | | ID NO: | ID | NO: | (SEQ ID |
| | | | | 141) | NO: | 138) | NO: 139) |
| | | | | | 137) | | |
| | Kabat | RASQSISNWV | RASTLQS | QQSHSL | DHYMH | TINPSGG | AADNGHAS |
| | | A (SEQ ID | (SEQ ID | PLT | (SEQ | RTSYAQK | GWLYYYGM |
| | | NO: 140) | NO: 217) | (SEQ | ID | FQG | DV (SEQ |
| | | | | ID NO: | NO: | (SEQ ID | ID NO: |
| | | | | 141) | 218) | NO: | 220) |
| | | | | | | 219) | |
| | IMGT | QSISNW | RAS | QQSHSL | GYTFS | INPSGGR | AADNGHAS |
| | | (SEQ ID | | PLT | DHY | T (SEQ | GWLYYYGM |
| | | NO: 221) | | (SEQ | (SEQ | ID NO: | DV (SEQ |
| | | | | ID NO: | ID | 223) | ID NO: |
| | | | | 141) | NO: | | 220) |
| | | | | | 222) | | |
| | CHOTHIA | RASQSISNWV | RASTLQS | QQSHSL | GYTFS | NPSGGR | NGHASGWL |
| | | A (SEQ ID | (SEQ ID | PLT | DH | (SEQ ID | YYYGMD |
| | | NO: 140) | NO: 217) | (SEQ | (SEQ | NO: | (SEQ ID |
| | | | | ID NO: | ID | 225) | NO: 226) |
| | | | | 141) | NO: | | |
| | | | | | 224) | | |
| PD1AB25 | Proprietary | KSSQSVLYSP | STRES | QQYYTT | FTFSN | SGITISG | CARGRGGS |
| | | NNKNYLA | (SEQ ID | PPT | SDMS | GSTYYA | GWLDYW |
| | | (SEQ ID | NO: 175) | (SEQ | (SEQ | (SEQ ID | (SEQ ID |
| | | NO: 174) | | ID NO: | ID | NO: | NO: 173) |
| | | | | 176) | NO: | 172) | |
| | | | | | 171) | | |
| | Kabat | KSSQSVLYSP | WASTRES | QQYYTT | NSDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 228) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | NSD | T (SEQ | WLDY |
| | | NO: 231) | | (SEQ | (SEQ | ID NO: | (SEQ ID |
| | | | | ID NO: | ID | 233) | NO: 230) |
| | | | | 176) | NO: | | |
| | | | | | 232) | | |
| | CHOTHIA | KSSQSVLYSP | WASTRES | QQYYTT | GFTFS | TISGGS | RGGSGWLD |
| | | NNKNYLA | (SEQ ID | PPT | NS | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 234) | | |
| PD1AB4 | Proprietary | QASRDIKNYL | SSLQS | QQSYST | YSFTT | GIINPSG | CASGWVYW |
| | | A (SEQ ID | (SEQ ID | PPT | YYMH | GSTSYA | (SEQ ID |
| | | NO: 40) | NO: 19) | (SEQ | (SEQ | (SEQ ID | NO: 39) |
| | | | | ID NO: | ID | NO: 38) | |
| | | | | 41) | NO: | | |
| | | | | | 37) | | |
| | Kabat | QASRDIKNYL | AASSLQS | QQSYST | TYYMH | IINPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 239) | |
| | IMGT | RDIKNY | AAS | QQSYST | GYSFT | INPSGGS | ASGWVY |
| | | (SEQ ID | | PPT | TYY | T (SEQ | (SEQ ID |
| | | NO: 241) | | (SEQ | (SEQ | ID NO: | NO: 240) |
| | | | | ID NO: | ID | 243) | |
| | | | | 41) | NO: | | |
| | | | | | 242) | | |
| | CHOTHIA | QASRDIKNYL | AASSLQS | QQSYST | GYSFT | NPSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 245) | |
| | | | | 41) | NO: | | |
| | | | | | 244) | | |
| PD1AB30 | Proprietary | KSSQSVLYSP | STRES | QQYYTT | FTFSN | SGITISG | CARGRGGS |
| | | NNKNYLA | (SEQ ID | PPT | SDMS | GSTYYA | GWLDYW |
| | | (SEQ ID | NO: 175) | (SEQ | (SEQ | (SEQ ID | (SEQ ID |
| | | NO: 174) | | ID NO: | ID | NO: | NO: 173) |
| | | | | 176) | NO: | 172) | |
| | | | | | 171) | | |
| | Kabat | KSSQSVLYSP | WASTRES | QQYYTT | NSDMS | GITISGG | ARGRGGSG |
| | | NNKNYLA | (SEQ ID | PPT | (SEQ | STYYADS | WLDY |
| | | (SEQ ID | NO: 227) | (SEQ | ID | VRG | (SEQ ID |
| | | NO: 174) | | ID NO: | NO: | (SEQ ID | NO: 230) |
| | | | | 176) | 228) | NO: | |
| | | | | | | 246) | |
| | IMGT | QSVLYSPNNK | WAS | QQYYTT | GFTFS | ITISGGS | ARGRGGSG |
| | | NY (SEQ ID | | PPT | NSD | T (SEQ | WLDY |
| | | NO: 231) | | (SEQ | (SEQ | ID NO: | (SEQ ID |
| | | | | ID NO: | ID | 233) | NO: 230) |
| | | | | 176) | NO: | | |
| | | | | | 232) | | |
| | CHOTHIA | KSSQSVLYSP | WASTRES | QQYYTT | GFTFS | TISGGS | RGGSGWLD |
| | | NNKNYLA | (SEQ ID | PPT | NS | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 234) | | |
| PD1AB17 | Proprietary | RASQGVGNAL | SSLQS | QQAYSF | YTFTD | GWMNTNS | CARGGSYS |
| | | G (SEQ ID | (SEQ ID | PWT | DYIH | GNTGYA | SGWYGRLD |
| | | NO: 118) | NO: 19) | (SEQ | (SEQ | (SEQ ID | YYYGMDVW |
| | | | | ID NO: | ID | NO: | (SEQ ID |
| | | | | 119) | NO: | 116) | NO: 117) |
| | | | | | 115) | | |
| | Kabat | RASQGVGNAL | AASSLQS | QQAYSF | DDYIH | WMNTNSG | ARGGSYSS |
| | | G (SEQ ID | (SEQ ID | PWT | (SEQ | NTGYAQK | GWYGRLDY |
| | | NO: 118) | NO: 237) | (SEQ | ID | FQG | YYGMDV |
| | | | | ID NO: | NO: | (SEQ ID | (SEQ ID |
| | | | | 119) | 247) | NO: | NO: 249) |
| | | | | | | 248) | |
| | IMGT | QGVGNA | AAS | QQAYSF | GYTFT | MNTNSGN | ARGGSYSS |
| | | (SEQ ID | | PWT | DDY | T (SEQ | GWYGRLDY |
| | | NO: 250) | | (SEQ | (SEQ | ID NO: | YYGMDV |
| | | | | ID NO: | ID | 252) | (SEQ ID |
| | | | | 119) | NO: | | NO: 249) |
| | | | | | 251) | | |
| | CHOTHIA | RASQGVGNAL | AASSLQS | QQAYSF | GYTFT | NTNSGN | GSYSSGWY |
| | | G (SEQ ID | (SEQ ID | PWT | DD | (SEQ ID | GRLDYYYG |
| | | NO: 118) | NO: 237) | (SEQ | (SEQ | NO: | MD (SEQ |
| | | | | ID NO: | ID | 254) | ID NO: |
| | | | | 119) | NO: | | 255) |
| | | | | | 253) | | |

Accordingly, in some embodiments, an antibody is provided that binds to PD-1 that comprises a LCDR set or a HCDR set as provided in the table above.

In some embodiments, the Fab CDRs of the following clones are provided, which are based on different formats that can be used to characterize CDRs.

PD-1 Antibody Fab Table 6

| | CDR | | | | | | |
|---|---|---|---|---|---|---|---|
| | Annotation | | | | | | |
| Clone | System | LCDR1 | LCDR2 | LCDR3 | HCDR1 | HCDR2 | HCDR3 |
| PD1AB43 | Kabat | QASRDIKNYL | AASSLQS | QQSYST | TYYMH | IIAPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 264) | |
| | IMGT | RDIKNY | AAS | QQSYST | GYSFT | IAPSGGS | ASGWVY |
| | | (SEQ ID | | PPT | TYY | T (SEQ | (SEQ ID |
| | | NO: 241) | | (SEQ | (SEQ | ID NO: | |
| | | | | ID NO: | ID | 265) | NO: 240) |
| | | | | 41) | NO: | | |
| | | | | | 242) | | |
| | CHOTHIA | QASRDIKNYL | AASSLQS | QQSYST | GYSFT | APSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 266) | |
| | | | | 41) | NO: | | |
| | | | | | 244) | | |
| PD1AB53 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB54 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB37 | Kabat | RASQSISSYL | AASSLQS | QQSYST | SYYMH | IINPSGG | ASGWVY |
| | | A (SEQ ID | (SEQ ID | PPT | (SEQ | STSYAQK | (SEQ ID |
| | | NO: 276) | NO: 237) | (SEQ | ID | FQG | NO: 240) |
| | | | | ID NO: | NO: | (SEQ ID | |
| | | | | 41) | 279) | NO: | |
| | | | | | | 239) | |
| | IMGT | QSISSY | AAS | QQSYST | GYSFT | INPSGGS | ASGWVY |
| | | (SEQ ID | | PPT | SYY | T (SEQ | (SEQ ID |
| | | NO: 271) | | (SEQ | (SEQ | ID NO: | NO: 240) |
| | | | | ID NO: | ID | 243) | |
| | | | | 41) | NO: | | |
| | | | | | 272) | | |
| | CHOTHIA | RASQSISSYL | AASSLQS | QQSYST | GYSFT | NPSGGS | WV |
| | | A (SEQ ID | (SEQ ID | PPT | SY | (SEQ ID | |
| | | NO: 276) | NO: 237) | (SEQ | (SEQ | NO: | |
| | | | | ID NO: | ID | 245) | |
| | | | | 41) | NO: | | |
| | | | | | 273) | | |
| PD1AB38 | Kabat | QASRDIKNYL | AASSLQS | QQSYST | TYYMH | IINPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 239) | |
| | IMGT | RDIKNY | AAS | QQSYST | GYSFT | INPSGGS | ASGWVY |
| | | (SEQ ID | | PPT | TYY | T (SEQ | (SEQ ID |
| | | NO: 241) | | (SEQ | (SEQ | ID NO: | NO: 240) |
| | | | | ID NO: | ID | 243) | |
| | | | | 41) | NO: | | |
| | | | | | 242) | | |
| | CHOTHIA | QASRDIKNYL | AASSLQS | QQSYST | GYSFT | NPSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 245) | |
| | | | | 41) | NO: | | |
| | | | | | 244) | | |
| PD1AB39 | Kabat | QASRDIKNYL | AASSLQS | QQSYST | TYYMH | IINPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 239) | |
| | IMGT | RDIKNY | AAS | QQSYST | GYSFT | INPSGGS | ASGWVY |
| | | (SEQ ID | | PPT | TYY | T (SEQ | (SEQ ID |
| | | NO: 241) | | (SEQ | (SEQ | ID NO: | NO: 240) |
| | | | | ID NO: | ID | 243) | |
| | | | | 41) | NO: | | |
| | | | | | 242) | | |
| | CHOTHIA | QASRDIKNYL | AASSLQS | QQSYST | GYSFT | NPSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 245) | |
| | | | | 41) | NO: | | |
| | | | | | 244) | | |
| PD1AB40 | Kabat | RASQSISSYL | AASSLQS | QQSYST | SYYMH | IINPSGG | ASGWVY |
| | | A (SEQ ID | (SEQ ID | PPT | | STSYAQK | (SEQ ID |
| | | NO: 276) | NO: 237) | (SEQ | | FQG | NO: 240) |
| | | | | ID NO: | | (SEQ ID | |
| | | | | 41) | | NO: | |
| | | | | | | 239) | |
| | IMGT | QSISSY | AAS | QQSYST | GYSFT | INPSGGS | ASGWVY |
| | | (SEQ ID | | PPT | SYY | T (SEQ | (SEQ ID |
| | | NO: 271) | | (SEQ | (SEQ | ID NO: | NO: 240) |
| | | | | ID NO: | ID | 243) | |
| | | | | 41) | NO: | | |
| | | | | | 272) | | |
| | CHOTHIA | RASQSISSYL | AASSLQS | QQSYST | GYSFT | NPSGGS | WV |
| | | A (SEQ ID | (SEQ ID | PPT | SY | (SEQ ID | |
| | | NO: 276) | NO: 237) | (SEQ | (SEQ | NO: | |
| | | | | ID NO: | ID | 245) | |
| | | | | 41) | NO: | | |
| | | | | | 273) | | |

Accordingly, in some embodiments, an antibody is provided that binds to PD-1 that comprises a Fab LCDR set or a Fab HCDR set as provided in a table herein.

In some embodiments, the scFv CDRs of the following clones are provided, which are based on different formats that can be used to characterize CDRs.

PD-1 Antibody scFv Table 7

| | CDR | | | | | | |
|---|---|---|---|---|---|---|---|
| | Annotation | | | | | | |
| Clone | System | LCDR1 | LCDR2 | LCDR3 | HCDR1 | HCDR2 | HCDR3 |
| PD1AB43 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB53 | Kabat | QASRDIKNYL | AASSLQS | QQSYST | TYYMH | IINPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 239) | |
| | IMGT | RDIKNY | AAS | QQSYST | GYSFT | INPSGGS | ASGWVY |
| | | (SEQ ID | | PPT | TYY | T (SEQ | (SEQ ID |
| | | NO: 241) | | (SEQ | (SEQ | ID NO: | NO: 240) |
| | | | | ID NO: | ID | 243) | |
| | | | | 41) | NO: | | |
| | | | | | 242) | | |
| | CHOTHIA | QASRDIKNYL | AASSLQS | QQSYST | GYSFT | NPSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 245) | |
| | | | | 41) | NO: | | |
| | | | | | 244) | | |
| PD1AB54 | Kabat | QASRDIKNYL | AASSLQS | QQSYST | TYYMH | IINPSGG | ASGWVY |
| | | A (SEQ ID | (SEQ ID | PPT | (SEQ | STSYAQK | (SEQ ID |
| | | NO: 40) | NO: 237) | (SEQ | ID | FQG | NO: 240) |
| | | | | ID NO: | NO: | (SEQ ID | |
| | | | | 41) | 238) | NO: | |
| | | | | | | 239) | |
| | IMGT | RDIKNY | AAS | QQSYST | GYSFT | INPSGGS | ASGWVY |
| | | (SEQ ID | | PPT | TYY | T (SEQ | (SEQ ID |
| | | NO: 241) | | (SEQ | (SEQ | ID NO: | NO: 240) |
| | | | | ID NO: | ID | 243) | |
| | | | | 41) | NO: | | |
| | | | | | 242) | | |
| | CHOTHIA | QASRDIKNYL | AASSLQS | QQSYST | GYSFT | NPSGGS | WV |
| | | A (SEQ ID | (SEQ ID | PPT | TY | (SEQ ID | |
| | | NO: 40) | NO: 237) | (SEQ | (SEQ | NO: | |
| | | | | ID NO: | ID | 245) | |
| | | | | 41) | NO: | | |
| | | | | | 244) | | |
| PD1AB37 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID NO: | NO: 227) | (SEQ | ID | VKG | (SEQ ID |
| | | 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB38 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB39 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |
| PD1AB40 | Kabat | KSSQSVLYSP | WASTRES | QQYYTT | SYDMS | GITISGG | ARGRGGSG |
| | | NNKNYLA | (SEQ ID | PPT | (SEQ | STYYADS | WLDY |
| | | (SEQ ID | NO: 227) | (SEQ | ID | VKG | (SEQ ID |
| | | NO: 174) | | ID NO: | NO: | (SEQ ID | NO: 230) |
| | | | | 176) | 267) | NO: | |
| | | | | | | 229) | |
| | IMGT | QSVLYSPNNK | WAS | QQYYTT | GFTFS | ITISGGS | ARGRGGSG |
| | | NY (SEQ ID | | PPT | SYD | (SEQ ID | WLDY |
| | | NO: 231) | | (SEQ | (SEQ | NO: | (SEQ ID |
| | | | | ID NO: | ID | 269) | NO: 230) |
| | | | | 176) | NO: | | |
| | | | | | 268) | | |
| | CHOTHIA | KSSQSVLYSP | WASTRES | QQYYTT | GFTFS | TISGGS | RGGSGWLD |
| | | NNKNYLA | (SEQ ID | PPT | SY | (SEQ ID | (SEQ ID |
| | | (SEQ ID | NO: 227) | (SEQ | (SEQ | NO: | NO: 236) |
| | | NO: 174) | | ID NO: | ID | 235) | |
| | | | | 176) | NO: | | |
| | | | | | 270) | | |

Accordingly, in some embodiments, an antibody is provided that binds to PD-1 that comprises a scFv LCDR set or a scFv HCDR set as provided in the tables herein. In some embodiments, an antibody is provided that binds to PD-1 that comprises a Fab LCDR and Fab HCDR set, or a scFv LCDR and a scFv HCDR set as provided in the PD-1 Antibody Fab Table 6 and PD-1 Antibody scFv Table 7 above.

In some embodiments, the PD-1 antibody comprises polypeptide selected from the following table:

PD-1 Antibody Table 8

| | Chain 1 | | | | | | | |
|---|---|---|---|---|---|---|---|---|
| | Fab VH | | Linker | scFv | Linker | scFv | Chain 2 | |
| Clone | Seq | CH1-CH3 Seq | Seq | VH Seq | Seq | VK Seq | VL Seq | CK Seq |
| PD1AB37 | EVQLVQS | ASTKGPSVFPLAP | GGGGSG | EVQLLE | GGGGSG | DIVMTQ | DIQMTQ | RTVAAPSV |
| | GAEVKKP | SSKSTSGGTAALG | GGGSGG | SGGGLV | GGGSGG | SPDSLA | SPSSLS | FIFPPSDE |
| | GASVKVS | CLVKDYFPEPVTV | GGS | QPGGSL | GGSGGG | VSLGER | ASVGDR | QLKSGTAS |
| | CKASGYS | SWNSGALTSGVHT | (SEQ | RLSCAA | GS | ATINCK | VTITCR | VVCLLNNF |
| | FTSYYMH | FPAVLQSSGLYSL | ID NO: | SGFTFS | (SEQ | SSQSVL | ASQSIS | YPREAKVQ |
| | WVRQAPG | SSVVTVPSSSLGT | 6) | SYDMSW | ID NO: | YSPNNK | SYLAWY | WKVDNALQ |
| | QGLEWMG | QTYICNVNHKPSN | | VRQAPG | 7) | NYLAWY | QQKPGK | SGNSQESV |
| | IINPSGG | TKVDKKVEPKSCD | | KGLEWV | | QQKPGQ | APKLLI | TEQDSKDS |
| | STSYAQK | KTHTCPPCPAPEA | | SGITIS | | PPKLLI | YAASSL | TYSLSSTL |
| | FQGRVTM | AGAPSVFLFPPKP | | GGSTYY | | YWASTR | QSGVPS | TLSKADYE |
| | TRDTSTS | KDTLMISRTPEVT | | ADSVKG | | ESGVPD | RFSGSG | KHKVYACE |
| | TVYMELS | CVVVDVSHEDPEV | | RFTISR | | RFSGSG | SGTDFT | VTHQGLSS |
| | SLRSEDT | KFNWYVDGVEVHN | | DNSKNT | | SGTDFT | LTISSL | PVTKSFNR |
| | AVYYCAS | AKTKPREEQYNST | | LYLQMN | | LTISSL | QPEDFA | GEC (SEQ |
| | GWVYWGQ | YRVVSVLTVLHQD | | SLRAED | | QAEDVA | TYYCQQ | ID NO: |
| | GTLVTVS | WLNGKEYKCKVSN | | TAVYYC | | VYYCQQ | SYSTPP | 274) |
| | S (SEQ | KALPAPIEKTISK | | ARGRGG | | YYTTPP | TFGPGT | |
| | ID NO: | AKGQPREPQVYTL | | SGWLDY | | TFGQGT | KVDIK | |
| | 256) | PPSREEMTKNQVS | | WGQGTT | | RLEIK | (SEQ | |
| | | LTCLVKGFYPSDI | | VTVSS | | (SEQ | ID NO: | |
| | | AVEWESNGQPENN | | (SEQ | | ID NO: | 259) | |
| | | YKTTPPVLDSDGS | | ID NO: | | 170) | | |
| | | FFLYSKLTVDKSR | | 257) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB38 | EVQLVQS | ASTKGPSVFPLAP | GGGGSG | EVQLLE | GGGGSG | DIVMTQ | DIQMTQ | RTVAAPSV |
| | GAEVKKP | SSKSTSGGTAALG | GGGSGG | SGGGLV | GGGSGG | SPDSLA | SPSSLS | FIFPPSDE |
| | GASVKVS | CLVKDYFPEPVTV | GGS | QPGGSL | GGSGGG | VSLGER | ASVGDR | QLKSGTAS |
| | CKASGYS | SWNSGALTSGVHT | (SEQ | RLSCAA | GS | ATINCK | VTITCQ | VVCLLNNF |
| | FTTYYMH | FPAVLQSSGLYSL | ID NO: | SGFTFS | (SEQ | SSQSVL | ASRDIK | YPREAKVQ |
| | WVRQAPG | SSVVTVPSSSLGT | 6) | SYDMSW | ID NO: | YSPNNK | NYLAWY | WKVDNALQ |
| | QGLEWMG | QTYICNVNHKPSN | | VRQAPG | 7) | NYLAWY | QQKPGK | SGNSQESV |
| | IINPSGG | TKVDKKVEPKSCD | | KGLEWV | | QQKPGQ | APKLLI | TEQDSKDS |
| | STSYAQK | KTHTCPPCPAPEA | | SGITIS | | PPKLLI | YAASSL | TYSLSSTL |
| | FQGRVTM | AGAPSVFLFPPKP | | GGSTYY | | YWASTR | QSGVPS | TLSKADYE |
| | TRDTSTS | KDTLMISRTPEVT | | ADSVKG | | ESGVPD | RFSGSG | KHKVYACE |
| | TVYMELS | CVVVDVSHEDPEV | | RFTISR | | RFSGSG | SGTDFT | VTHQGLSS |
| | SLRSEDT | KFNWYVDGVEVHN | | DNSKNT | | SGTDFT | LTISSL | PVTKSFNR |
| | AVYYCAS | AKTKPREEQYNST | | LYLQMN | | LTISSL | QPEDFA | GEC (SEQ |
| | GWVYWGQ | YRVVSVLTVLHQD | | SLRAED | | QAEDVA | TYYCQQ | ID NO: |
| | GTLVTVS | WLNGKEYKCKVSN | | TAVYYC | | VYYCQQ | SYSTPP | 274) |
| | S (SEQ | KALPAPIEKTISK | | ARGRGG | | YYTTPP | TFGPGT | |
| | ID NO: | AKGQPREPQVYTL | | SGWLDY | | TFGQGT | KVDIK | |
| | 275) | PPSREEMTKNQVS | | WGQGTT | | RLEIK | (SEQ ID | |
| | | LTCLVKGFYPSDI | | VTVSS | | (SEQ | NO: | |
| | | AVEWESNGQPENN | | (SEQ | | ID NO: | 36) | |
| | | YKTTPPVLDSDGS | | ID NO: | | 170) | | |
| | | FFLYSKLTVDKSR | | 257) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB39 | EVQLVQS | ASTKGPSVFPLAP | GGGGSG | EVQLLE | GGGGSG | DIVMTQ | DIQMTQ | RTVAAPSV |
| | GAEVKKP | SSKSTSGGTAALG | GGGSGG | SGGGLV | GGGSGG | SPDSLA | SPSSLS | FIFPPSDE |
| | GASVKVS | CLVKDYFPEPVTV | GGS | QPGGSL | GGSGGG | VSLGER | ASVGDR | QLKSGTAS |
| | CKASGYS | SWNSGALTSGVHT | (SEQ | RLSCAA | GS | ATINCK | VTITCQ | VVCLLNNF |
| | FTTYYMH | FPAVLQSSGLYSL | ID NO: | SGFTFS | (SEQ | SSQSVL | ASRDIK | YPREAKVQ |
| | WVRQAPG | SSVVTVPSSSLGT | 6) | SYDMSW | ID NO: | YSPNNK | NYLAWY | WKVDNALQ |
| | QGLEWMG | QTYICNVNHKPSN | | VRQAPG | 7) | NYLAWY | QQKPGK | SGNSQESV |
| | IINPSGG | TKVDKKVEPKSCD | | KCLEWV | | QQKPGQ | APKLLI | TEQDSKDS |
| | STSYAQK | KTHTCPPCPAPEA | | SGITIS | | PPKLLI | YAASSL | TYSLSSTL |
| | FQGRVTM | AGAPSVFLFPPKP | | GGSTYY | | YWASTR | QSGVPS | TLSKADYE |
| | TRDTSTS | KDTLMISRTPEVT | | ADSVKG | | ESGVPD | RFSGSG | KHKVYACE |
| | TVYMELS | CVVVDVSHEDPEV | | RFTISR | | RFSGSG | SGTDFT | VTHQGLSS |
| | SLRSEDT | KFNWYVDGVEVHN | | DNSKNT | | SGTDFT | LTISSL | PVTKSFNR |
| | AVYYCAS | AKTKPREEQYNST | | LYLQMN | | LTISSL | QPEDFA | GEC (SEQ |
| | GWVYWGQ | YRVVSVLTVLHQD | | SLRAED | | QAEDVA | TYYCQQ | ID NO: |
| | GTLVTVS | WLNGKEYKCKVSN | | TAVYYC | | VYYCQQ | SYSTPP | 274) |
| | S (SEQ | KALPAPIEKTISK | | ARGRGG | | YYTTPP | TFGPGT | |
| | ID NO: | AKGQPREPQVYTL | | SGWLDY | | TFGCGT | KVDIK | |
| | 275) | PPSREEMTKNQVS | | WGQGTT | | RLEIK | (SEQ ID | |
| | | LTCLVKGFYPSDI | | VTVSS | | (SEQ | NO: | |
| | | AVEWESNGQPENN | | (SEQ | | ID NO: | 36) | |
| | | YKTTPPVLDSDGS | | ID NO: | | 279) | | |
| | | FFLYSKLTVDKSR | | 278) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB40 | EVQLVQS | ASTKGPSVFPLAP | GGGGSG | EVQLLE | GGGGSG | DIVMTQ | DIQMTQ | RTVAAPSV |
| | GAEVKKP | SSKSTSGGTAALG | GGGSGG | SGGGLV | GGGSGG | SPDSLA | SPSSLS | FIFPPSDE |
| | GASVKVS | CLVKDYFPEPVTV | GGS | QPGGSL | GGSGGG | VSLGER | ASVGDR | QLKSGTAS |
| | CKASGYS | SWNSGALTSGVHT | (SEQ | RLSCAA | GS | ATINCK | VTITCR | VVCLLNNF |
| | FTSYYMH | FPAVLQSSGLYSL | ID NO: | SGFTFS | (SEQ | SSQSVL | ASQSIS | YPREAKVQ |
| | WVRQAPG | SSVVTVPSSSLGT | 6) | SYDMSW | ID NO: | YSPNNK | SYLAWY | WKVDNALQ |
| | QGLEWMG | QTYICNVNHKPSN | | VRQAPG | 7) | NYLAWY | QQKPGK | SGNSQESV |
| | IINPSGG | TKVDKKVEPKSCD | | KCLEWV | | QQKPGQ | APKLLI | TEQDSKDS |
| | STSYAQK | KTHTCPPCPAPEA | | SGITIS | | PPKLLI | YAASSL | TYSLSSTL |
| | FQGRVTM | AGAPSVFLFPPKP | | GGSTYY | | YWASTR | QSGVPS | TLSKADYE |
| | TRDTSTS | KDTLMISRTPEVT | | ADSVKG | | ESGVPD | RFSGSG | KHKVYACE |
| | TVYMELS | CVVVDVSHEDPEV | | RFTISR | | RFSGSG | SGTDFT | VTHQGLSS |
| | SLRSEDT | KFNWYVDGVEVHN | | DNSKNT | | SGTDFT | LTISSL | PVTKSFNR |
| | AVYYCAS | AKTKPREEQYNST | | LYLQMN | | LTISSL | QPEDFA | GEC (SEQ |
| | GWVYWGQ | YRVVSVLTVLHQD | | SLRAED | | QAEDVA | TYYCQQ | ID NO: |
| | GTLVTVS | WLNGKEYKCKVSN | | TAVYYC | | VYYCQQ | SYSTPP | 274) |
| | S (SEQ | KALPAPIEKTISK | | ARGRGG | | YYTTPP | TFGPGT | |
| | ID NO: | AKGQPREPQVYTL | | SGWLDY | | TFGCGT | KVDIK | |
| | 256) | PPSREEMTKNQVS | | WGQGTT | | RLEIK | (SEQ | |
| | | LTCLVKGFYPSDI | | VTVSS | | (SEQ | ID NO: | |
| | | AVEWESNGQPENN | | (SEQ | | ID NO: | 259) | |
| | | YKTTPPVLDSDGS | | ID NO: | | 279) | | |
| | | FFLYSKLTVDKSR | | 278) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB41 | EVQLLES | ASTKGPSVFPLAP | GGGGSG | EVQLLE | GGGGSG | DIVMTQ | DIVMTQ | RTVAAPSV |
| | GGGLVQP | SSKSTSGGTAALG | GGGSGG | SGGGLV | GGGSGG | SPDSLA | SPDSLA | FIFPPSDE |
| | GGSLRLS | CLVKDYFPEPVTV | GGS | QPGGSL | GGSGGG | VSLGER | VSLGER | QLKSGTAS |
| | CAASGFT | SWNSGALTSGVHT | (SEQ | RLSCAA | GS | ATINCK | ATINCK | VVCLLNNF |
| | FSSYDMS | FPAVLQSSGLYSL | ID NO: | SGFTFS | (SEQ | SSQSVL | SSQSVL | YPREAKVQ |
| | WVRQAPG | SSVVTVPSSSLGT | 6) | SYDMSW | ID NO: | YSPNNK | YSPNNK | WKVDNALQ |
| | KGLEWVS | QTYICNVNHKPSN | | VRQAPG | 7) | NYLAWY | NYLAWY | SGNSQESV |
| | GITISGG | TKVDKKVEPKSCD | | KGLEWV | | QQKPGQ | QQKPGQ | TEQDSKDS |
| | STYYADS | KTHTCPPCPAPEA | | SGITIS | | PPKLLI | PPKLLI | TYSLSSTL |
| | VKGRFTI | AGAPSVFLFPPKP | | GGSTYY | | YWASTR | YWASTR | TLSKADYE |
| | SRDNSKN | KDTLMISRTPEVT | | ADSVKG | | ESGVPD | ESGVPD | KHKVYACE |
| | TLYLQMN | CVVVDVSHEDPEV | | RFTISR | | RFSGSG | RFSGSG | VTHQGLSS |
| | SLRAEDT | KFNWYVDGVEVHN | | DNSKNT | | SGTDFT | SGTDFT | PVTKSFNR |
| | AVYYCAR | AKTKPREEQYNST | | LYLQMN | | LTISSL | LTISSL | GEC (SEQ |
| | GRGGSGW | YRVVSVLTVLHQD | | SLRAED | | QAEDVA | QAEDVA | ID NO: |
| | LDYWGQG | WLNGKEYKCKVSN | | TAVYYC | | VYYCQQ | VYYCQQ | 274) |
| | TTVTVSS | KALPAPIEKTISK | | ARGRGG | | YYTTPP | YYTTPP | |
| | (SEQ ID | AKGQPREPQVYTL | | SGWLDY | | TFGQGT | TFGQGT | |
| | NO: | PPSREEMTKNQVS | | WGQGTT | | RLEIK | RLEIK | |
| | 257) | LTCLVKGFYPSDI | | VTVSS | | (SEQ | (SEQ | |
| | | AVEWESNGQPENN | | (SEQ | | ID NO: | ID NO: | |
| | | YKTTPPVLDSDGS | | ID NO: | | 170) | 170) | |
| | | FFLYSKLTVDKSR | | 257) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB42 | QVQLVQS | ASTKGPSVFPLAP | GGGGSG | QVQLVQ | GGGGSG | DIQMTQ | DIQMTQ | RTVAAPSV |
| | GAEVKKP | SSKSTSGGTAALG | GGGSGG | SGAEVK | GGGSGG | SPSSLS | SPSSLS | FIFPPSDE |
| | GASVKVS | CLVKDYFPEPVTV | GGS | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 6) | TYYMHW | ID NO: | NYLAWY | NYLAWY | WKVDNALQ |
| | QGLEWMG | QTYICNVNHKPSN | | VRQAPG | 7) | QQKPGK | QQKPGK | SGNSQESV |
| | IIAPSGG | TKVDKKVEPKSCD | | QGLEWM | | APKLLI | APKLLI | TEQDSKDS |
| | STSYAQK | KTHTCPPCPAPEA | | GIIAPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 202) | PPSREEMTKNQVS | | VTVSS | | (SEQ ID | (SEQ ID | |
| | | LTCLVKGFYPSDI | | (SEQ ID | | NO: | NO: | |
| | | AVEWESNGQPENN | | NO: | | 36) | 36) | |
| | | YKTTPPVLDSDGS | | 202) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB43 | QVQLVQS | ASTKGPSVFPLAP | GGGGSG | EVQLLE | GGGGSG | DIVMTQ | DIQMTQ | RTVAAPSV |
| | GAEVKKP | SSKSTSGGTAALG | GGGSGG | SGGGLV | GGGSGG | SPDSLA | SPSSLS | FIFPPSDE |
| | GASVKVS | CLVKDYFPEPVTV | GGS | QPGGSL | GGSGGG | VSLGER | ASVGDR | QLKSGTAS |
| | CKASGYS | SWNSGALTSGVHT | (SEQ | RLSCAA | GS | ATINCK | VTITCQ | VVCLLNNF |
| | FTTYYMH | FPAVLQSSGLYSL | ID NO: | SGFTFS | (SEQ | SSQSVL | ASRDIK | YPREAKVQ |
| | WVRQAPG | SSVVTVPSSSLGT | 6) | SYDMSW | ID NO: | YSPNNK | NYLAWY | WKVDNALQ |
| | QGLEWMG | QTYICNVNHKPSN | | VRQAPG | 7) | NYLAWY | QQKPGK | SGNSQESV |
| | IIAPSGG | TKVDKKVEPKSCD | | KGLEWV | | QQKPGQ | APKLLI | TEQDSKDS |
| | STSYAQK | KTHTCPPCPAPEA | | SGITIS | | PPKLLI | YAASSL | TYSLSSTL |
| | FQGRVTM | AGAPSVFLFPPKP | | GGSTYY | | YWASTR | QSGVPS | TLSKADYE |
| | TRDTSTS | KDTLMISRTPEVT | | ADSVKG | | ESGVPD | RFSGSG | KHKVYACE |
| | TVYMELS | CVVVDVSHEDPEV | | RFTISR | | RFSGSG | SGTDFT | VTHQGLSS |
| | SLRSEDT | KFNWYVDGVEVHN | | DNSKNT | | SGTDFT | LTISSL | PVTKSFNR |
| | AVYYCAS | AKTKPREEQYNST | | LYLQMN | | LTISSL | QPEDFA | GEC (SEQ |
| | GWVYWGQ | YRVVSVLTVLHQD | | SLRAED | | QAEDVA | TYYCQQ | ID NO: |
| | GTLVTVS | WLNGKEYKCKVSN | | TAVYYC | | VYYCQQ | SYSTPP | 274) |
| | S (SEQ | KALPAPIEKTISK | | ARGRGG | | YYTTPP | TFGPGT | |
| | ID NO: | AKGQPREPQVYTL | | SGWLDY | | TFGQGT | KVDIK | |
| | 202) | PPSREEMTKNQVS | | WGQGTT | | RLEIK | (SEQ | |
| | | LTCLVKGFYPSDI | | VTVSS | | (SEQ | ID NO: | |
| | | AVEWESNGQPENN | | (SEQ | | ID NO: | 36) | |
| | | YKTTPPVLDSDGS | | ID NO: | | 170) | | |
| | | FFLYSKLTVDKSR | | 257) | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB44 | EVQLLES | ASTKGPSVFPLAP | GGGGSG | QVQLVQ | GGGGSG | DIQMTQ | DIVMTQ | RTVAAPSV |
| | GGGLVQP | SSKSTSGGTAALG | GGGSGG | SGAEVK | GGGSGG | SPSSLS | SPDSLA | FIFPPSDE |
| | GGSLRLS | CLVKDYFPEPVTV | GGS | KPGASV | GGSGGG | ASVGDR | VSLGER | QLKSGTAS |
| | CAASGFT | SWNSGALTSGVHT | (SEQ | KVSCKA | GS | VTITCQ | ATINCK | VVCLLNNF |
| | FSSYDMS | FPAVLQSSGLYSL | ID NO: | SGYSFT | (SEQ | ASRDIK | SSQSVL | YPREAKVQ |
| | WVRQAPG | SSVVTVPSSSLGT | 6) | TYYMHW | ID NO: | NYLAWY | YSPNNK | WKVDNALQ |
| | KGLEWVS | QTYICNVNHKPSN | | VRQAPG | 7) | QQKPGK | NYLAWY | SGNSQESV |
| | GITISGG | TKVDKKVEPKSCD | | QGLEWM | | APKLLI | QQKPGQ | TEQDSKDS |
| | STYYADS | KTHTCPPCPAPEA | | GIIAPS | | YAASSL | PPKLLI | TYSLSSTL |
| | VKGRFTI | AGAPSVFLFPPKP | | GGSTSY | | QSGVPS | YWASTR | TLSKADYE |
| | SRDNSKN | KDTLMISRTPEVT | | AQKFQG | | RFSGSG | ESGVPD | KHKVYACE |
| | TLYLQMN | CVVVDVSHEDPEV | | RVTMTR | | SGTDFT | RFSGSG | VTHQGLSS |
| | SLRAEDT | KFNWYVDGVEVHN | | DTSTST | | LTISSL | SGTDFT | PVTKSFNR |
| | AVYYCAR | AKTKPREEQYNST | | VYMELS | | QPEDFA | LTISSL | GEC (SEQ |
| | GRGGSGW | YRVVSVLTVLHQD | | SLRSED | | TYYCQQ | QAEDVA | ID NO: |
| | LDYWGQG | WLNGKEYKCKVSN | | TAVYYC | | SYSTPP | VYYCQQ | 274) |
| | TTVTVSS | KALPAPIEKTISK | | ASGWVY | | TFGPGT | YYTTPP | |
| | (SEQ ID | AKGQPREPQVYTL | | WGQGTL | | KVDIK | TFGQGT | |
| | NO: | PPSREEMTKNQVS | | VTVSS | | (SEQ | RLEIK | |
| | 257) | LTCLVKGFYPSDI | | (SEQ ID | | ID NO: | (SEQ | |
| | | AVEWESNGQPENN | | NO: | | 36) | ID NO: | |
| | | YKTTPPVLDSDGS | | 202) | | | 170) | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB45 | EVQLVQS | ASTKGPSVFPLAP | AAAEKA | EVQLVQ | GGGGSG | DIQMTQ | DIQMTQ | RTVAAPSV |
| | GAEVKKP | SSKSTSGGTAALG | AAEKAA | SGAEVK | GGGSGG | SPSSLS | SPSSLS | FIFPPSDE |
| | GASVKVS | CLVKDYFPEPVTV | AEK | KPGASV | GGS | ASVGDR | ASVGDR | QLKSGTAS |
| | CKASGYS | SWNSGALTSGVHT | (SEQ | KVSCKA | (SEQ | VTITCQ | VTITCQ | VVCLLNNF |
| | FTTYYMH | FPAVLQSSGLYSL | ID NO: | SGYSFT | ID NO: | ASRDIK | ASRDIK | YPREAKVQ |
| | WVRQAPG | SSVVTVPSSSLGT | 277) | TYYMHW | 6) | NYLAWY | NYLAWY | WKVDNALQ |
| | QGLEWMG | QTYICNVNHKPSN | | VRQAPG | | QQKPGK | QQKPGK | SGNSQESV |
| | IINPSGG | TKVDKKVEPKSCD | | QGLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 275) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB46 | EVQLVQS | ASTKGPSVFPLAP | AAAEKA | EVQLVQ | GGGGSG | DIQMTQ | DIQMTQ | RTVAAPSV |
| | GAEVKKP | SSKSTSGGTAALG | AAEKAA | SGAEVK | GGGSGG | SPSSLS | SPSSLS | FIFPPSDE |
| | GASVKVS | CLVKDYFPEPVTV | AEK | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 277) | TYYMHW | ID NO: | NYLAWY | NYLAWY | WKVDNALQ |
| | QGLEWMG | QTYICNVNHKPSN | | VRQAPG | 7) | QQKPGK | QQKPGK | SGNSQESV |
| | IINPSGG | TKVDKKVEPKSCD | | QCLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 262) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB47 | EVQLVQS | ASTKGPSVFPLAP | AAAEKA | EVQLVQ | GGGGSG | DIQMTQ | DIQMTQ | RTVAAPSV |
| | GAEVKKP | SSKSTSGGTAALG | AAEKAA | SGAEVK | GGGSGG | SPSSLS | SPSSLS | FIFPPSDE |
| | GASVKVS | CLVKDYFPEPVTV | AEK | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 277) | TYYMHW | ID NO: | NYLAWY | NYLAWY | WKVDNALQ |
| | QGLEWMG | QTYICNVNHKPSN | | VRQAPG | 7) | QQKPGK | QQKPGK | SGNSQESV |
| | IINPSGG | TKVDKKVEPKSCD | | QGLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 275) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB48 | EVQLVQS | ASTKGPSVFPLAP | GGGSEG | EVQLVQ | GGGGSG | DIQMTQ | DIQMTQ | RTVAAPSV |
| | GAEVKKP | SSKSTSGGTAALG | GGSEGG | SGAEVK | GGGSGG | SPSSLS | SPSSLS | FIFPPSDE |
| | GASVKVS | CLVKDYFPEPVTV | GSE | KPGASV | GGS | ASVGDR | ASVGDR | QLKSGTAS |
| | CKASGYS | SWNSGALTSGVHT | (SEQ | KVSCKA | (SEQ | VTITCQ | VTITCQ | VVCLLNNF |
| | FTTYYMH | FPAVLQSSGLYSL | ID NO: | SGYSFT | ID NO: | ASRDIK | ASRDIK | YPREAKVQ |
| | WVRQAPG | SSVVTVPSSSLGT | 1) | TYYMHW | 6) | NYLAWY | NYLAWY | WKVDNALQ |
| | QGLEWMG | QTYICNVNHKPSN | | VRQAPG | | QQKPGK | QQKPGK | SGNSQESV |
| | IINPSGG | TKVDKKVEPKSCD | | QGLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 275) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB49 | EVQLVQS | ASTKGPSVFPLAP | GGGSEG | EVQLVQ | GGGGSG | DIQMTQ | DIQMTQ | RTVAAPSV |
| | GAEVKKP | SSKSTSGGTAALG | GGSEGG | SGAEVK | GGGSGG | SPSSLS | SPSSLS | FIFPPSDE |
| | GASVKVS | CLVKDYFPEPVTV | GSE | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 1) | TYYMHW | ID NO: | NYLAWY | NYLAWY | WKVDNALQ |
| | QGLEWMG | QTYICNVNHKPSN | | VRQAPG | 7) | QQKPGK | QQKPGK | SGNSQESV |
| | IINPSGG | TKVDKKVEPKSCD | | QCLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 262) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB50 | EVQLVQS | ASTKGPSVFPLAP | GGGSEG | EVQLVQ | GGGGSG | DIQMTQ | DIQMTQ | RTVAAPSV |
| | GAEVKKP | SSKSTSGGTAALG | GGSEGG | SGAEVK | GGGSGG | SPSSLS | SPSSLS | FIFPPSDE |
| | GASVKVS | CLVKDYFPEPVTV | GSE | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 1) | TYYMHW | ID NO: | NYLAWY | NYLAWY | WKVDNALQ |
| | QGLEWMG | QTYICNVNHKPSN | | VRQAPG | | QQKPGK | QQKPGK | SGNSQESV |
| | IINPSGG | TKVDKKVEPKSCD | | QGLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 275) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB51 | EVQLVQS | ASTKGPSVFPLAP | GGGGSG | EVQLVQ | GGGGSG | DIQMTQ | DIQMTQ | RTVAAPSV |
| | GAEVKKP | SSKSTSGGTAALG | GGGSGG | SGAEVK | GGGSGG | SPSSLS | SPSSLS | FIFPPSDE |
| | GASVKVS | CLVKDYFPEPVTV | GGS | KPGASV | GGS | ASVGDR | ASVGDR | QLKSGTAS |
| | CKASGYS | SWNSGALTSGVHT | (SEQ | KVSCKA | (SEQ | VTITCQ | VTITCQ | VVCLLNNF |
| | FTTYYMH | FPAVLQSSGLYSL | ID NO: | SGYSFT | ID NO: | ASRDIK | ASRDIK | YPREAKVQ |
| | WVRQAPG | SSVVTVPSSSLGT | 6) | TYYMHW | 6 | NYLAWY | NYLAWY | WKVDNALQ |
| | QGLEWMG | QTYICNVNHKPSN | | VRQAPG | | QQKPGK | QQKPGK | SGNSQESV |
| | IINPSGG | TKVDKKVEPKSCD | | QGLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 275) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |
| PD1AB52 | EVQLVQS | ASTKGPSVFPLAP | GGGGSG | EVQLVQ | GGGGSG | DIQMTQ | DIQMTQ | RTVAAPSV |
| | GAEVKKP | SSKSTSGGTAALG | GGGSGG | SGAEVK | GGGSGG | SPSSLS | SPSSLS | FIFPPSDE |
| | GASVKVS | CLVKDYFPEPVTV | GGS | KPGASV | GGSGGG | ASVGDR | ASVGDR | QLKSGTAS |
| | CKASGYS | SWNSGALTSGVHT | (SEQ | KVSCKA | GS | VTITCQ | VTITCQ | VVCLLNNF |
| | FTTYYMH | FPAVLQSSGLYSL | ID NO: | SGYSFT | (SEQ | ASRDIK | ASRDIK | YPREAKVQ |
| | WVRQAPG | SSVVTVPSSSLGT | 6) | TYYMHW | ID NO: | NYLAWY | NYLAWY | WKVDNALQ |
| | QGLEWMG | QTYICNVNHKPSN | | VRQAPG | 7) | QQKPGK | QQKPGK | SGNSQESV |
| | IINPSGG | TKVDKKVEPKSCD | | QCLEWM | | APKLLI | APKLLI | TEQDSKDS |
| | STSYAQK | KTHTCPPCPAPEA | | GIINPS | | YAASSL | YAASSL | TYSLSSTL |
| | FQGRVTM | AGAPSVFLFPPKP | | GGSTSY | | QSGVPS | QSGVPS | TLSKADYE |
| | TRDTSTS | KDTLMISRTPEVT | | AQKFQG | | RFSGSG | RFSGSG | KHKVYACE |
| | TVYMELS | CVVVDVSHEDPEV | | RVTMTR | | SGTDFT | SGTDFT | VTHQGLSS |
| | SLRSEDT | KFNWYVDGVEVHN | | DTSTST | | LTISSL | LTISSL | PVTKSFNR |
| | AVYYCAS | AKTKPREEQYNST | | VYMELS | | QPEDFA | QPEDFA | GEC (SEQ |
| | GWVYWGQ | YRVVSVLTVLHQD | | SLRSED | | TYYCQQ | TYYCQQ | ID NO: |
| | GTLVTVS | WLNGKEYKCKVSN | | TAVYYC | | SYSTPP | SYSTPP | 274) |
| | S (SEQ | KALPAPIEKTISK | | ASGWVY | | TFGPGT | TFGPGT | |
| | ID NO: | AKGQPREPQVYTL | | WGQGTL | | KVDIK | KVDIK | |
| | 275) | PPSREEMTKNQVS | | VTVSS | | (SEQ | (SEQ | |
| | | LTCLVKGFYPSDI | | (SEQ | | ID NO: | ID NO: | |
| | | AVEWESNGQPENN | | ID NO: | | 36) | 36) | |
| | | YKTTPPVLDSDGS | | 262) | | | | |
| | | FFLYSKLTVDKSR | | | | | | |
| | | WQQGNVFSCSVMH | | | | | | |
| | | EALHNHYTQKSLS | | | | | | |
| | | LSPG (SEQ ID | | | | | | |
| | | NO: 12) | | | | | | |

In some embodiments, the PD-1 antibody comprises a polypeptide selected from the PD-1 Antibody Table 8.

In some embodiments, the PD-1 antibody comprises a polypeptide selected from the following table:

PD-1 Antibody Table 9

| | Chain 1 | | | | | | Chain 2 | |
|---|---|---|---|---|---|---|---|---|
| | Fab VH | | Linker | scFv VK | Linker | ScFv VH | | CK |
| Clone | Seq | CH1—CH3 Seq | Seq | Seq | Seq | Seq | VL Seq | Seq |
| PD1AB | EVQLLE | ASTKGPSVFPLAPS | GGGSEG | DIQMTQSP | GGGGSG | EVQLVQS | DIVMTQS | RTVAA |
| 53 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 261) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 54 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QGLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGPGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 36) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 275) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | AAAEKA | DIQMTQSP | GGGGSG | EVQLVQS | DIQMTQS | RTVAA |
| 55 | SGAEVK | SKSTSGGTAALGCL | AAEKAA | SSLSASVG | GGGSGG | GAEVKKP | PSSLSAS | PSVFI |
| | KPGASV | VKDYFPEPVTVSWN | AEK | DRVTITCQ | GGS | GASVKVS | VGDRVTI | FPPSD |
| | KVSCKA | SGALTSGVHTFPAV | (SEQ | ASRDIKNY | (SEQ | CKASGYS | TCQASRD | EQLKS |
| | SGYSFT | LQSSGLYSLSSVVT | ID NO: | LAWYQQKP | ID NO: | FTTYYMH | IKNYLAW | GTASV |
| | TYYMHW | VPSSSLGTQTYICN | 277) | GKAPKLLI | 6) | WVRQAPG | YQQKPGK | VCLLN |
| | VRQAPG | VNHKPSNTKVDKKV | | YAASSLQS | | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | AAAEKA | DIQMTQSP | GGGGSG | EVQLVQS | DIQMTQS | RTVAA |
| 56 | SGAEVK | SKSTSGGTAALGCL | AAEKAA | SSLSASVG | GGGSGG | GAEVKKP | PSSLSAS | PSVFI |
| | KPGASV | VKDYFPEPVTVSWN | AEK | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 277) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGCGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 261) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | AAAEKA | DIQMTQSP | GGGGSG | EVQLVQS | DIQMTQS | RTVAA |
| 57 | SGAEVK | SKSTSGGTAALGCL | AAEKAA | SSLSASVG | GGGSGG | GAEVKKP | PSSLSAS | PSVFI |
| | KPGASV | VKDYFPEPVTVSWN | AEK | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 277) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGSEG | DIQMTQSP | GGGGSG | EVQLVQS | DIQMTQS | RTVAA |
| 58 | SGAEVK | SKSTSGGTAALGCL | GGSEGG | SSLSASVG | GGGSGG | GAEVKKP | PSSLSAS | PSVFI |
| | KPGASV | VKDYFPEPVTVSWN | GSE | DRVTITCQ | GGS | GASVKVS | VGDRVTI | FPPSD |
| | KVSCKA | SGALTSGVHTFPAV | (SEQ | ASRDIKNY | (SEQ | CKASGYS | TCQASRD | EQLKS |
| | SGYSFT | LQSSGLYSLSSVVT | ID NO: | LAWYQQKP | ID NO: | FTTYYMH | IKNYLAW | GTASV |
| | TYYMHW | VPSSSLGTQTYICN | 1) | GKAPKLLI | 6) | WVRQAPG | YQQKPGK | VCLLN |
| | VRQAPG | VNHKPSNTKVDKKV | | YAASSLQS | | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGSEG | DIQMTQSP | GGGGSG | EVQLVQS | DIQMTQS | RTVAA |
| 59 | SGAEVK | SKSTSGGTAALGCL | GGSEGG | SSLSASVG | GGGSGG | GAEVKKP | PSSLSAS | PSVFI |
| | KPGASV | VKDYFPEPVTVSWN | GSE | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 1) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGCGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 261) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGSEG | DIQMTQSP | GGGGSG | EVQLVQS | DIQMTQS | RTVAA |
| 60 | SGAEVK | SKSTSGGTAALGCL | GGSEGG | SSLSASVG | GGGSGG | GAEVKKP | PSSLSAS | PSVFI |
| | KPGASV | VKDYFPEPVTVSWN | GSE | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 1) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGGSG | DIQMTQSP | GGGGSG | EVQLVQS | DIQMTQS | RTVAA |
| 61 | SGAEVK | SKSTSGGTAALGCL | GGGSGG | SSLSASVG | GGGSGG | GAEVKKP | PSSLSAS | PSVFI |
| | KPGASV | VKDYFPEPVTVSWN | GGS | DRVTITCQ | GGS | GASVKVS | VGDRVTI | FPPSD |
| | KVSCKA | SGALTSGVHTFPAV | (SEQ | ASRDIKNY | (SEQ | CKASGYS | TCQASRD | EQLKS |
| | SGYSFT | LQSSGLYSLSSVVT | ID NO: | LAWYQQKP | ID NO: | FTTYYMH | IKNYLAW | GTASV |
| | TYYMHW | VPSSSLGTQTYICN | 6) | GKAPKLLI | 6) | WVRQAPG | YQQKPGK | VCLLN |
| | VRQAPG | VNHKPSNTKVDKKV | | YAASSLQS | | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGGSG | DIQMTQSP | GGGGSG | EVQLVQS | DIQMTQS | RTVAA |
| 62 | SGAEVK | SKSTSGGTAALGCL | GGGSGG | SSLSASVG | GGGSGG | GAEVKKP | PSSLSAS | PSVFI |
| | KPGASV | VKDYFPEPVTVSWN | GGS | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 6) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGCGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 261) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLVQ | ASTKGPSVFPLAPS | GGGGSG | DIQMTQSP | GGGGSG | EVQLVQS | DIQMTQS | RTVAA |
| 63 | SGAEVK | SKSTSGGTAALGCL | GGGSGG | SSLSASVG | GGGSGG | GAEVKKP | PSSLSAS | PSVFI |
| | KPGASV | VKDYFPEPVTVSWN | GGS | DRVTITCQ | GGSGGG | GASVKVS | VGDRVTI | FPPSD |
| | KVSCKA | SGALTSGVHTFPAV | (SEQ | ASRDIKNY | GS | CKASGYS | TCQASRD | EQLKS |
| | SGYSFT | LQSSGLYSLSSVVT | ID NO: | LAWYQQKP | (SEQ | FTTYYMH | IKNYLAW | GTASV |
| | TYYMHW | VPSSSLGTQTYICN | 6) | GKAPKLLI | ID NO: | WVRQAPG | YQQKPGK | VCLLN |
| | VRQAPG | VNHKPSNTKVDKKV | | YAASSLQS | 7) | QGLEWMG | APKLLIY | NFYPR |
| | QGLEWM | EPKSCDKTHTCPPC | | GVPSRFSG | | IINPSGG | AASSLQS | EAKVQ |
| | GIINPS | PAPEAAGAPSVFLF | | SGSGTDFT | | STSYAQK | GVPSRFS | WKVDN |
| | GGSTSY | PPKPKDTLMISRTP | | LTISSLQP | | FQGRVTM | GSGSGTD | ALQSG |
| | AQKFQG | EVTCVVVDVSHEDP | | EDFATYYC | | TRDTSTS | FTLTISS | NSQES |
| | RVTMTR | EVKFNWYVDGVEVH | | QQSYSTPP | | TVYMELS | LQPEDFA | VTEQD |
| | DTSTST | NAKTKPREEQYNST | | TFGPGTKV | | SLRSEDT | TYYCQQS | SKDST |
| | VYMELS | YRVVSVLTVLHQDW | | DIK (SEQ | | AVYYCAS | YSTPPTF | YSLSS |
| | SLRSED | LNGKEYKCKVSNKA | | ID NO: | | GWVYWGQ | GPGTKVD | TLTLS |
| | TAVYYC | LPAPIEKTISKAKG | | 36) | | GTLVTVS | IK (SEQ | KADYE |
| | ASGWVY | QPREPQVYTLPPSR | | | | S (SEQ | ID NO: | KHKVY |
| | WGQGTL | EEMTKNQVSLTCLV | | | | ID NO: | 36) | ACEVT |
| | VTVSS | KGFYPSDIAVEWES | | | | 275) | | HQGLS |
| | (SEQ | NGQPENNYKTTPPV | | | | | | SPVTK |
| | ID NO: | LDSDGSFFLYSKLT | | | | | | SFNRG |
| | 275) | VDKSRWQQGNVFSC | | | | | | EC |
| | | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 64 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDE | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 280) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 65 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GAAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 282) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 66 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GSAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 283) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 67 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GNAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 284) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 68 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GNAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 285) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 69 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDILNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 286) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 70 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DTVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDET | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 287) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 71 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GAAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 288) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 72 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DTVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 289) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 73 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GSAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 290) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 74 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLNSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 261) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 291) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 75 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDINNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 292) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 262) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 76 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLNSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 280) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 293) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 77 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSILSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 294) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | EVQLVQS | DIVMTQS | RTVAA |
| 78 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSINSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 295) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 281) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | QVQLVQS | DIVMTQS | RTVAA |
| 79 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCR | GGSGGG | GASVKVS | LGERATI | FPPSD |
| | RLSCAA | SGALTSGVHTFPAV | (SEQ | ASQSISSY | GS | CKASGYS | NCKSSQS | EQLKS |
| | SGFTFS | LQSSGLYSLSSVVT | ID NO: | LAWYQQKP | (SEQ | FTSYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 280) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 296) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID NO: |
| | | ID NO: 12) | | | | | | 274) |
| PD1AB | EVQLLE | ASTKGPSVFPLAPS | GGGSEG | DIQMTQSP | GGGGSG | QVQLVQS | DIVMTQS | RTVAA |
| 80 | SGGGLV | SKSTSGGTAALGCL | GGSEGG | SSLSASVG | GGGSGG | GAEVKKP | PDSLAVS | PSVFI |
| | QPGGSL | VKDYFPEPVTVSWN | GSE | DRVTITCQ | GGSGGG | GASVKVS | LGERATI | FPPSD |
| | RLSCAA | SGALTSGVHTFPAV | (SEQ | ASRDIKNY | GS | CKASGYS | NCKSSQS | EQLKS |
| | SGFTFS | LQSSGLYSLSSVVT | ID NO: | LAWYQQKP | (SEQ | FTTYYMH | VLYSPNN | GTASV |
| | SYDMSW | VPSSSLGTQTYICN | 1) | GKAPKLLI | ID NO: | WVRQAPG | KNYLAWY | VCLLN |
| | VRQAPG | VNHKPSNTKVDKKV | | YAASSLQS | 7) | QCLEWMG | QQKPGQP | NFYPR |
| | KGLEWV | EPKSCDKTHTCPPC | | GVPSRFSG | | IINPSGG | PKLLIYW | EAKVQ |
| | SGITIS | PAPEAAGAPSVFLF | | SGSGTDFT | | STSYAQK | ASTRFSG | WKVDN |
| | GGSTYY | PPKPKDTLMISRTP | | LTISSLQP | | FQGRVTM | VPDRFSG | ALQSG |
| | ADSVKG | EVTCVVVDVSHEDP | | EDFATYYC | | TRDTSTS | SGSGTDF | NSQES |
| | RFTISR | EVKFNWYVDGVEVH | | QQSYSTPP | | TVYMELS | TLTISSL | VTEQD |
| | DNSKNT | NAKTKPREEQYNST | | TFGCGTKV | | SLRSEDT | QAEDVAV | SKDST |
| | LYLQMN | YRVVSVLTVLHQDW | | DIK (SEQ | | AVYYCAS | YYCQQYY | YSLSS |
| | SLRAED | LNGKEYKCKVSNKA | | ID NO: | | GWVYWGQ | TTPPTFG | TLTLS |
| | TAVYYC | LPAPIEKTISKAKG | | 261) | | GTLVTVS | QGTRLEI | KADYE |
| | ARGRGG | QPREPQVYTLPPSR | | | | S (SEQ | K (SEQ | KHKVY |
| | SGWLDY | EEMTKNQVSLTCLV | | | | ID NO: | ID NO: | ACEVT |
| | WGQGTT | KGFYPSDIAVEWES | | | | 297) | 170) | HQGLS |
| | VTVSS | NGQPENNYKTTPPV | | | | | | SPVTK |
| | (SEQ | LDSDGSFFLYSKLT | | | | | | SFNRG |
| | ID NO: | VDKSRWQQGNVFSC | | | | | | EC |
| | 257) | SVMHEALHNHYTQK | | | | | | (SEQ |
| | | SLSLSPG (SEQ | | | | | | ID |
| | | ID NO: 12) | | | | | | NO: |
| | | | | | | | | 274) |

In some embodiments, the PD-1 antibody comprises a polypeptide selected from the PD-1 Antibody Table 9.

In some embodiments, the PD-1 antibody comprises a polypeptide selected from the following table:

PD-1 Antibody Table 10

| | Fab VH | | Linker | Human Serum | | |
|---|---|---|---|---|---|---|
| Clone | Seq | CH1—CH3 Seq | Seq | Albumin | VL Seq | CK Seq |
| PD1AB8 | EVQLVQSGA | ASTKGPSVFPLAPSSKSTSG | GGGSEG | DAHKSEVAHRF | DIQMTQSP | RTVAAPSV |
| 1 | EVKKPGASV | GTAALGCLVKDYFPEPVTVS | GGSEGG | KDLGEENFKAL | SSLSASVG | FIFPPSDE |
| | KVSCKASGY | WNSGALTSGVHTFPAVLQSS | GSE | VLIAFAQYLQQ | DRVTITCQ | QLKSGTAS |
| | SFTTYYMHW | GLYSLSSVVTVPSSSLGTQT | (SEQ | CPFEDHVKLVN | ASRDIKNY | VVCLLNNF |
| | VRQAPGQGL | YICNVNHKPSNTKVDKKVEP | ID NO: | EVTEFAKTCVA | LAWYQQKP | YPREAKVQ |
| | EWMGIINPS | KSCDKTHTCPPCPAPEAAGA | 1) | DESAENCDKSL | GKAPKLLI | WKVDNALQ |
| | GGSTSYAQK | PSVFLFPPKPKDTLMISRTP | | HTLFGDKLCTV | YAASSLQS | SGNSQESV |
| | FQGRVTMTR | EVTCVVVDVSHEDPEVKENW | | ATLRETYGEMA | GVPSRFSG | TEQDSKDS |
| | DTSTSTVYM | YVDGVEVHNAKTKPREEQYN | | DCCAKQEPERN | SGSGTDFT | TYSLSSTL |
| | ELSSLRSED | STYRVVSVLTVLHQDWLNGK | | ECFLQHKDDNP | LTISSLQP | TLSKADYE |
| | TAVYYCASG | EYKCKVSNKALPAPIEKTIS | | NLPRLVRPEVD | EDFATYYC | KHKVYACE |
| | WVYWGQGTL | KAKGQPREPQVYTLPPSREE | | VMCTAFHDNEE | QQSYSTPP | VTHQGLSS |
| | VTVSS | MTKNQVSLTCLVKGFYPSDI | | TFLKKYLYEIA | TFGPGTKV | PVTKSFNR |
| | (SEQ ID | AVEWESNGQPENNYKTTPPV | | RRHPYFYAPEL | DIK (SEQ | GEC (SEQ |
| | NO: 275) | LDSDGSFFLYSKLTVDKSRW | | LFFAKRYKAAF | ID NO: | ID NO: |
| | | QQGNVFSCSVMHEALHNHYT | | TECCQAADKAA | 36) | 274) |
| | | QKSLSLSPG (SEQ ID | | CLLPKLDELRD | | |
| | | NO: 12) | | EGKASSAKQR | | |
| | | | | (SEQ ID NO: | | |
| | | | | 298) | | |
| PD1AB8 | EVQLVQSGA | ASTKGPSVFPLAPSSKSTSG | GGGSEG | DAHKSEVAHRF | DIQMTQSP | RTVAAPSV |
| 2 | EVKKPGASV | GTAALGCLVKDYFPEPVTVS | GGSEGG | KDLGEENFKAL | SSLSASVG | FIFPPSDE |
| | KVSCKASGY | WNSGALTSGVHTFPAVLQSS | GSE | VLIAFAQYLQQ | DRVTITCQ | QLKSGTAS |
| | SFTTYYMHW | GLYSLSSVVTVPSSSLGTQT | (SEQ | CPFEDHVKLVN | ASRDIKNY | VVCLLNNF |
| | VRQAPGQGL | YICNVNHKPSNTKVDKKVEP | ID NO: | EVTEFAKTCVA | LAWYQQKP | YPREAKVQ |
| | EWMGIINPS | KSCDKTHTCPPCPAPEAAGA | 1) | DESAENCDKSL | GKAPKLLI | WKVDNALQ |
| | GGSTSYAQK | PSVFLFPPKPKDTLMISRTP | | HTLFGDKLCTV | YAASSLQS | SGNSQESV |
| | FQGRVTMTR | EVTCVVVDVSHEDPEVKENW | | ATLRETYGEMA | GVPSRFSG | TEQDSKDS |
| | DTSTSTVYM | YVDGVEVHNAKTKPREEQYN | | DCCAKQEPERN | SGSGTDFT | TYSLSSTL |
| | ELSSLRSED | STYRVVSVLTVLHQDWLNGK | | ECFLQHKDDNP | LTISSLQP | TLSKADYE |
| | TAVYYCASG | EYKCKVSNKALPAPIEKTIS | | NLPRLVRPEVD | EDFATYYC | KHKVYACE |
| | WVYWGQGTL | KAKGQPREPQVYTLPPSREE | | VMCTAFHDNEE | QQSYSTPP | VTHQGLSS |
| | VTVSS | MTKNQVSLTCLVKGFYPSDI | | TFLKKYLYEIA | TFGPGTKV | PVTKSENR |
| | (SEQ ID | AVEWESNGQPENNYKTTPPV | | RRHPYFYAPEL | DIK (SEQ | GEC (SEQ |
| | NO: 275) | LDSDGSFFLYSKLTVDKSRW | | LFFAKRYKAAF | ID NO: | ID NO: |
| | | QQGNVFSCSVMHEALHNHYT | | TECCQAADKAA | 36) | 274) |
| | | QKSLSLSPG (SEQ ID | | CLLPKLDELRD | | |
| | | NO: 12) | | EGKASSAKQR | | |
| | | | | (SEQ ID NO: | | |
| | | | | 298) | | |

In some embodiments, the PD-1 antibody comprises a polypeptide selected from the PD-1 Antibody Table 10.

In some embodiments, the PD-1 antibody comprises a polypeptide selected from the following table:

PD-1 Antibody Table 11

| | Fab VH | | Linker | | | |
|---|---|---|---|---|---|---|
| Clone | Seq | CH1—CH3 Seq | Seq | scFv VH Seq | VL Seq | CK Seq |
| PD1AB8 | EVQLVQSGA | ASTKGPSVFPLAPSSKSTSG | GGGGSG | EVQLVQSGAEV | DIQMTQSP | RTVAAPSV |
| 3 | EVKKPGASV | GTAALGCLVKDYFPEPVTVS | GGGSGG | KKPGASVKVSC | SSLSASVG | FIFPPSDE |
| | KVSCKASGY | WNSGALTSGVHTFPAVLQSS | GGS | KASGYSFTTYY | DRVTITCQ | QLKSGTAS |
| | SFTTYYMHW | GLYSLSSVVTVPSSSLGTQT | (SEQ | MHWVRQAPGQG | ASRDIKNY | VVCLLNNF |
| | VRQAPGQGL | YICNVNHKPSNTKVDKKVEP | ID NO: | LEWMGIINPSG | LAWYQQKP | YPREAKVQ |
| | EWMGIINPS | KSCDKTHTCPPCPAPEAAGA | 6) | GSTSYAQKFQG | GKAPKLLI | WKVDNALQ |
| | GGSTSYAQK | PSVFLFPPKPKDTLMISRTP | | RVTMTRDTSTS | YAASSLQS | SGNSQESV |
| | FQGRVTMTR | EVTCVVVDVSHEDPEVKENW | | TVYMELSSLRS | GVPSRFSG | TEQDSKDS |
| | DTSTSTVYM | YVDGVEVHNAKTKPREEQYN | | EDTAVYYCASG | SGSGTDFT | TYSLSSTL |
| | ELSSLRSED | STYRVVSVLTVLHQDWLNGK | | WVYWGQGTLVT | LTISSLQP | TLSKADYE |
| | TAVYYCASG | EYKCKVSNKALPAPIEKTIS | | VSSASTKGPSV | EDFATYYC | KHKVYACE |
| | WVYWGQGTL | KAKGQPREPQVYTLPPSREE | | FPLAPSSKSTS | QQSYSTPP | VTHQGLSS |
| | VTVSS | MTKNQVSLTCLVKGFYPSDI | | GGTAALGCLVK | TFGPGTKV | PVTKSFNR |
| | (SEQ ID | AVEWESNGQPENNYKTTPPV | | DYFPEPVTVSW | DIK (SEQ | GEC (SEQ |
| | NO: 275) | LDSDGSFFLYSKLTVDKSRW | | NSGALTSGVHT | ID NO: | ID NO: |
| | | QQGNVFSCSVMHEALHNHYT | | FPAVLQSSGLY | 36) | 274) |
| | | QKSLSLSPG (SEQ ID | | SLSSVVTVPSS | | |
| | | NO: 12) | | SLGTQTYICNV | | |
| | | | | NHKPSNTKVDK | | |
| | | | | KVEPKSCDKTH | | |
| | | | | T (SEQ ID | | |
| | | | | NO: 299) | | |
| PD1AB8 | EVQLVQSGA | ASTKGPSVFPLAPSSKSTSG | GGGGSG | EVQLVQSGAEV | DIQMTQSP | RTVAAPSV |
| 4 | EVKKPGASV | GTAALGCLVKDYFPEPVTVS | GGGSGG | KKPGASVKVSC | SSLSASVG | FIFPPSDE |
| | KVSCKASGY | WNSGALTSGVHTFPAVLQSS | GGS | KASGYSFTTYY | DRVTITCQ | QLKSGTAS |
| | SFTTYYMHW | GLYSLSSVVTVPSSSLGTQT | (SEQ | MHWVRQAPGQG | ASRDIKNY | VVCLLNNE |
| | VRQAPGQGL | YICNVNHKPSNTKVDKKVEP | ID NO: | LEWMGIINPSG | LAWYQQKP | YPREAKVQ |
| | EWMGIINPS | KSCDKTHTCPPCPAPEAAGA | 6) | GSTSYAQKFQG | GKAPKLLI | WKVDNALQ |
| | GGSTSYAQK | PSVFLFPPKPKDTLMISRTP | | RVTMTRDTSTS | YAASSLQS | SGNSQESV |
| | FQGRVTMTR | EVTCVVVDVSHEDPEVKENW | | TVYMELSSLRS | GVPSRFSG | TEQDSKDS |
| | DTSTSTVYM | YVDGVEVHNAKTKPREEQYN | | EDTAVYYCASG | SGSGTDFT | TYSLSSTL |
| | ELSSLRSED | STYRVVSVLTVLHQDWLNGK | | WVYWGQGTLVT | LTISSLQP | TLSKADYE |
| | TAVYYCASG | EYKCKVSNKALPAPIEKTIS | | VSSASTKGPSV | EDFATYYC | KHKVYACE |
| | WVYWGQGTL | KAKGQPREPQVYTLPPSREE | | FPLAPSSKSTS | QQSYSTPP | VTHQGLSS |
| | VTVSS | MTKNQVSLTCLVKGFYPSDI | | GGTAALGCLVK | TFGPGTKV | PVTKSENR |
| | (SEQ ID | AVEWESNGQPENNYKTTPPV | | DYFPEPVTVSW | DIK (SEQ | GEC (SEQ |
| | NO: 275) | LDSDGSFFLYSKLTVDKSRW | | NSGALTSGVHT | ID NO: | ID NO: |
| | | QQGNVFSCSVMHEALHNHYT | | FPAVLQSSGLY | 36) | 274) |
| | | QKSLSLSPG (SEQ ID | | SLSSVVTVPSS | | |
| | | NO: 12) | | SLGTQTYICNV | | |
| | | | | NHKPSNTKVDK | | |
| | | | | KVEPKSCDKTH | | |
| | | | | T (SEQ ID | | |
| | | | | NO: 299) | | |
| PD1AB8 | EVQLVQSGA | ASTKGPSVFPLAPSSKSTSG | GGGGSG | EVQLVQSGAEV | DIQMTQSP | RTVAAPSV |
| 5 | EVKKPGASV | GTAALGCLVKDYFPEPVTVS | GGGSGG | KKPGASVKVSC | SSLSASVG | FIFPPSDE |
| | KVSCKASGY | WNSGALTSGVHTFPAVLQSS | GGS | KASGYSFTTYY | DRVTITCQ | QLKSGTAS |
| | SFTTYYMHW | GLYSLSSVVTVPSSSLGTQT | (SEQ | MHWVRQAPGQG | ASRDIKNY | VVCLLNNF |
| | VRQAPGQGL | YICNVNHKPSNTKVDKKVEP | ID NO: | LEWMGIINPSG | LAWYQQKP | YPREAKVQ |
| | EWMGIINPS | KSCDKTHTCPPCPAPEAAGA | 6) | GSTSYAQKFQG | GKAPKLLI | WKVDNALQ |
| | GGSTSYAQK | PSVFLFPPKPKDTLMISRTP | | RVTMTRDTSTS | YAASSLQS | SGNSQESV |
| | FQGRVTMTR | EVTCVVVDVSHEDPEVKENW | | TVYMELSSLRS | GVPSRFSG | TEQDSKDS |
| | DTSTSTVYM | YVDGVEVHNAKTKPREEQYN | | EDTAVYYCASG | SGSGTDFT | TYSLSSTL |
| | ELSSLRSED | STYRVVSVLTVLHQDWLNGK | | WVYWGQGTLVT | LTISSLQP | TLSKADYE |
| | TAVYYCASG | EYKCKVSNKALPAPIEKTIS | | VSSASTKGPSV | EDFATYYC | KHKVYACE |
| | WVYWGQGTL | KAKGQPREPQVYTLPPSREE | | FPLAPSSKSTS | QQSYSTPP | VTHQGLSS |
| | VTVSS | MTKNQVSLTCLVKGFYPSDI | | GGTAALGCLVK | TFGPGTKV | PVTKSFNR |
| | (SEQ ID | AVEWESNGQPENNYKTTPPV | | DYFPEPVTVSW | DIK (SEQ | GEC (SEQ |
| | NO: 275) | LDSDGSFFLYSKLTVDKSRW | | NSGALTSGVHT | ID NO: | ID NO: |
| | | QQGNVFSCSVMHEALHNHYT | | FPAVLQSSGLY | 36) | 274) |
| | | QKSLSLSPG (SEQ ID | | SLSSVVTVPSS | | |
| | | NO: 12) | | SLGTQTYICNV | | |
| | | | | NHKPSNTKVDK | | |
| | | | | KVEPKSCDKTH | | |
| | | | | T (SEQ ID | | |
| | | | | NO: 299) | | |

In some embodiments, the PD-1 antibody comprises a polypeptide selected from the PD-1 Antibody Table 11.

As provided for herein, the antibody or polypeptide can have a plurality of polypeptide chains as provided in the tables herein, such as two of chain 1 and two of chain 2 to make a paratopic molecule. These are non-limiting examples.

In some embodiments, a polypeptide is provided that comprises a plurality of antibodies that bind to PD-1. A plurality of antibodies comprises more than one antibody that have the same or different CDR regions.

In some embodiments, the PD-1 antibody comprises a sequence as shown in PD-1 Antibody Table 4. In some embodiments, the antibody is in a scFV format as illustrated in the PD-1 Antibody Table 4. In some embodiments, the antibody comprises a CDR1 from any one of clones of the PD-1 Antibody Table 4, a CDR2 from any one of clones of the PD-1 Antibody Table 4, and a CDR3 from any one of clones of the PD-1 Antibody Table 4. In some embodiments, the antibody comprises a LCDR1 from any one of clones of the PD-1 Antibody Table 4, a LCDR2 from any one of clones of the PD-1 Antibody Table 4, and a LCDR3 from any one of clones of the PD-1 Antibody Table 4. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the PD-1 antibody has a VH region selected from any one of clones of the PD-1 Antibody Table 4 and a VL region selected from any one of clones as set forth in the PD-1 Antibody Table 4.

In some embodiments, the molecule comprises an antibody that binds to PD-1. In some embodiments, the antibody comprises (i) a heavy chain variable region comprising heavy chain CDR1, CDR2, and CDR3 sequences, wherein the heavy chain CDR1 sequence has the amino acid sequence of any of the CDR1 sequences set forth in PD-1 Antibody Table 4; the heavy chain CDR2 has the amino acid sequence of any of the CDR2 sequences set forth in PD-1 Antibody Table 4, and the heavy chain CDR3 has the amino acid sequence of any of the CDR3 sequences set forth in PD-1 Antibody Table 4; or variants of any of the foregoing; and (ii) a light chain variable region comprising light chain CDR1, CDR2, and CDR3 sequences, wherein the light chain CDR1 sequence has the amino acid sequence of any of the LCDR1 sequences set forth in PD-1 Antibody Table 4; the light chain LCDR2 has the amino acid sequence of any of the LCDR2 sequences set forth in PD-1 Antibody Table 4, and the light chain CDR3 has the amino acid sequence of any of the LCDR3 sequences set forth in PD-1 Antibody Table 4, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in PD1AB4 of PD-1 Antibody Table 4, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in PD1AB4 of PD-1 Antibody Table 4, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in PD1AB30 of PD-1 Antibody Table 4, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in PD1AB30 of PD-1 Antibody Table 4, or variants of any of the foregoing.

These are non-limiting illustrative examples and the antibodies can have the CDRs as set forth in the tables provided herein and are explicitly referenced without writing out the previous paragraphs for each CDR set.

In some embodiments, the PD-1 antibody comprises a VH and VL(VK) chain as provided herein, such as those listed in the PD-1 Antibody Table 4.

In some embodiments, the PD-1 antibody comprises a sequence as shown in PD-1 Antibody Fab Table 6 and PD-1 Antibody scFv Table 7. In some embodiments, the antibody is in a Fab format as illustrated in the PD-1 Antibody Fab Table 6. In some embodiments, the antibody is in a scFv format as illustrated in the PD-1 Antibody scFv Table 7. In some embodiments, the antibody comprises a CDR1 from any one of clones of the PD-1 Antibody Fab Table 6 and PD-1 Antibody scFv Table 7, a CDR2 from any one of clones of the PD-1 Antibody Fab Table 6 and PD-1 Antibody scFv Table 7, and a CDR3 from any one of clones of the PD-1 Antibody Fab Table 6 and PD-1 Antibody scFv Table 7. In some embodiments, the antibody comprises a LCDR1 from any one of clones of the PD-1 Antibody Fab Table 6 and PD-1 Antibody scFv Table 7, a LCDR2 from any one of clones of the PD-1 Antibody Fab Table 6 and PD-1 Antibody scFv Table 7, and a LCDR3 from any one of clones of the PD-1 Antibody Fab Table 6 and PD-1 Antibody scFv Table 7. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the PD-1 antibody has a VH region selected from any one of clones of the PD-1 Antibody Table 8 and PD-1 Antibody Table 9 and a VL region selected from any one of clones as set forth in the PD-1 Antibody Table 8 and PD-1 Antibody Table 9.

In some embodiments, the molecule comprises an antibody that binds to PD-1. In some embodiments, the antibody comprises (i) a heavy chain variable region comprising heavy chain CDR1, CDR2, and CDR3 sequences, wherein the heavy chain CDR1 sequence has the amino acid sequence of any of the CDR1 sequences set forth in PD-1 Antibody Fab Table 6 and PD-1 Antibody scFv Table 7; the heavy chain CDR2 has the amino acid sequence of any of the CDR2 sequences set forth in PD-1 Antibody Fab Table 6 and PD-1 Antibody scFv Table 7, and the heavy chain CDR3 has the amino acid sequence of any of the CDR3 sequences set forth in PD-1 Antibody Fab Table 6 and PD-1 Antibody scFv Table 7; or variants of any of the foregoing; and (ii) a light chain variable region comprising light chain CDR1, CDR2, and CDR3 sequences, wherein the light chain CDR1 sequence has the amino acid sequence of any of the LCDR1 sequences set forth in PD-1 Antibody Fab Table 6 and PD-1 Antibody scFv Table 7; the light chain LCDR2 has the amino acid sequence of any of the LCDR2 sequences set forth in PD-1 Antibody Fab Table 6 and PD-1 Antibody scFv Table 7, and the light chain CDR3 has the amino acid sequence of any of the LCDR3 sequences set forth in PD-1 Antibody Fab Table 6 and PD-1 Antibody scFv Table 7, or variants of any of the foregoing.

In some embodiments, the antibody comprises a Fab heavy chain variable region comprising Fab heavy chain CDR1, CDR2, and CDR3 sequences, wherein the Fab heavy chain CDR1, CDR2, and CDR3 sequences have the amino acid sequence as set forth in PD1AB37 of PD-1 Antibody Fab Table 6, or variants of any of the foregoing; and (ii) a Fab light chain variable region comprising light chain CDR1, CDR2, and CDR3 sequences, wherein the Fab light chain CDR1, CDR2, and CDR3 sequences have the amino acid sequence as set forth sequence as set forth in PD1AB37 of PD-1 Antibody Fab Table 6, or variants of any of the foregoing.

In some embodiments, the antibody comprises a scFv heavy chain variable region comprising scFv heavy chain CDR1, CDR2, and CDR3 sequences, wherein the scFv heavy chain CDR1, CDR2, and CDR3 sequences have the amino acid sequence as set forth in PD1AB37 of PD-1 Antibody scFv Table 7, or variants of any of the foregoing; and (ii) a scFv light chain variable region comprising scFv light chain CDR1, CDR2, and CDR3 sequences, wherein the scFv light chain CDR1, CDR2, and CDR3 sequences have the amino acid sequence as set forth sequence as set forth in PD1AB37 of PD-1 Antibody scFv Table 7, or variants of any of the foregoing.

In some embodiments, the antibody comprises a Fab heavy chain variable region comprising Fab heavy chain CDR1, CDR2, and CDR3 sequences, wherein the Fab heavy chain CDR1, CDR2, and CDR3 sequences have the amino acid sequence as set forth in PD1AB53 of PD-1 Antibody Fab Table 6, or variants of any of the foregoing; and (ii) a Fab light chain variable region comprising light chain CDR1, CDR2, and CDR3 sequences, wherein the Fab light chain CDR1, CDR2, and CDR3 sequences have the amino acid sequence as set forth sequence as set forth in PD1AB53 of PD-1 Antibody Fab Table 6, or variants of any of the foregoing.

In some embodiments, the antibody comprises a scFv heavy chain variable region comprising scFv heavy chain CDR1, CDR2, and CDR3 sequences, wherein the scFv heavy chain CDR1, CDR2, and CDR3 sequences have the amino acid sequence as set forth in PD1AB53 of PD-1 Antibody scFv Table 7, or variants of any of the foregoing; and (ii) a scFv light chain variable region comprising scFv light chain CDR1, CDR2, and CDR3 sequences, wherein the scFv light chain CDR1, CDR2, and CDR3 sequences have the amino acid sequence as set forth sequence as set forth in PD1AB53 of PD-1 Antibody scFv Table 7, or variants of any of the foregoing.

These are non-limiting illustrative examples and the antibodies can have the CDRs as set forth in the tables provided herein and are explicitly referenced without writing out the previous paragraphs for each CDR set.

In some embodiments, the PD-1 antibody comprises a Fab VH and VL(VK) chain as provided herein, such as those listed in the PD-1 Antibody Table 8 and PD-1 Antibody Table 9. In some embodiments, the Fab VH peptide comprises a sequence of SEQ ID NO: 256 or 260. In some embodiments, the Fab VK chain comprises a sequence of SEQ ID NO: 259 or 263. In some embodiments, the antibody comprises a Fab VH of SEQ ID NO: 256 and a Fab VK of SEQ ID NO: 259. In some embodiments, the antibody comprises a Fab VH of SEQ ID NO: 260 and a Fab VK of SEQ ID NO: 263. In some embodiments, the PD-1 antibody comprises a scFv VH and VL(VK) chain as provided herein, such as those listed in the PD-1 Antibody Table 8 and PD-1 Antibody Table 9. In some embodiments, the scFv VH peptide comprises a sequence of SEQ ID NO: 257 or 261. In some embodiments, the scFv VK chain comprises a sequence of SEQ ID NO: 258 or 262. In some embodiments, the antibody comprises a scFv VH of SEQ ID NO: 257 and a scFv VK of SEQ ID NO: 258. In some embodiments, the antibody comprises a scFv VH of SEQ ID NO: 261 and a scFv VK of SEQ ID NO: 262.

In some embodiments, the PD-1 antibody comprises a Fab VH and VL(VK) and scFv VH and VL(VK) chain as provided herein, such as those listed in the PD-1 Antibody Table 8 and PD-1 Antibody Table 9. In some embodiments, the Fab VH peptide comprises a sequence of SEQ ID NO: 256 or 260 and the scFv VH peptide comprises a sequence of SEQ ID NO: 257 or 261. In some embodiments, the Fab VK chain comprises a sequence of SEQ ID NO: 259 or 263, and the scFv VK chain comprises a sequence of SEQ ID NO: 258 or 262. In some embodiments, the antibody comprises a Fab VH of SEQ ID NO: 256 and a Fab VK of SEQ ID NO: 259 and a scFv VH of SEQ ID NO: 257 and a scFv VK of SEQ ID NO: 258. In some embodiments, the antibody comprises a Fab VH of SEQ ID NO: 260 and a Fab VK of SEQ ID NO: 263 and a scFv VH of SEQ ID NO: 261 and a scFv VK of SEQ ID NO: 262.

In some embodiments, the scFv comprises from the N- to C-terminus VH and VL. In some embodiments, from the N- to C-terminus VH is linked to VL via a linker. In some embodiments, the linker is any linker provided herein. In some embodiments, the scFv comprises the N- to C-terminus VL and VH. In some embodiments, from the N- to C-terminus VL is linked to VH via a linker. In some embodiments, the linker is any linker provided herein.

In some embodiments, the PD-1 antibody comprises a sequence as shown in PD-1 Antibody Table 4. In some embodiments, the antibody is in a scFV format. In some embodiments, the antibody comprises a VH sequence from any one of clones of PD-1 Antibody Table 4. In some embodiments, the antibody comprises a VK sequence from any one of clones of the PD-1 Antibody Table 4. In some embodiments, the amino acid residues of the VH or VK shown above contain mutations. In some embodiments, the VH or VK contain 1, 2, 3, 4, 5, 6, 7, 8, 9, or 10 substitutions or mutations. In some embodiments, the substitution is a conservative substitution.

In some embodiments, if therapeutic compound comprises a Fc portion, the Fc domain, (portion) bears mutations to render the Fc region “effectorless” that is unable to bind FcRs. The mutations that render Fc regions effectorless are known. In some embodiments, the mutations in the Fc region, which is according to the known numbering system, are selected from the group consisting of: K322A, L234A, L235A, G237A, L234F, L235E, N297, P331S, or any combination thereof. In some embodiments, the Fc mutations comprises a mutation at L234 and/or L235 and/or G237. In some embodiments, the Fc mutations comprise L234A and/or L235A mutations, which can be referred to as LALA mutations. In some embodiments, the Fc mutations comprise L234A, L235A, and G237A mutations.

Disclosed herein are Linker polypeptides, therapeutic peptides, and nucleic acids encoding the polypeptides (e.g., therapeutic compounds), vectors comprising the nucleic acid sequences, and cells comprising the nucleic acids or vectors.

The FAb (20) and (25) and scFV (50) and (55) domains as illustrated in FIG. 1 and FIG. 2 can comprise any of the heavy and light chains or CDR sequences, alone, or as provided as a set forth in the tables and as described herein and above.

Pharmaceutical Compositions and Kits

In embodiments, the present embodiments provide compositions, e.g., pharmaceutically acceptable compositions, which include a therapeutic compound or polypeptide provided for herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g., by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating, and coloring agents can be used. The carriers can be used in pharmaceutical compositions comprising therapeutic compounds provided for herein.

The compositions and compounds of the embodiments provided herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, therapeutic molecule is administered by intravenous infusion or injection. In another embodiment, therapeutic molecule is administered by intramuscular or subcutaneous injection. In another embodiment, therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

The pharmaceutical compositions typically should be sterile and, in some embodiments, stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of therapeutic compound can be determined by a skilled artisan. In certain embodiments, therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, therapeutic compound is administered at a dose from about 10 to 20 mg/kg every other week. Therapeutic compound can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, therapeutic compound can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a polypeptide. A “therapeutically effective amount” refers to an amount effective, at dosages and for periods of time necessary, to achieve the desired therapeutic result. A therapeutically effective amount of a polypeptide may vary according to factors such as the disease state, age, sex, and weight of the individual, and the ability of therapeutic compound to elicit a desired response in the individual. A therapeutically effective amount is also one in which any toxic or detrimental effects of a therapeutic molecule t is outweighed by therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

In some embodiments, embodiments provided herein also include, but are not limited to:

1. A polypeptide comprising first, second, third, and fourth binding domains that bind to PD-1, wherein the first and second binding domains bind to a first epitope on PD-1 and the third and fourth binding domains bind to a second epitope on PD-1, wherein the first and second epitope are not the same.
2. The polypeptide of embodiment 1, wherein the polypeptide is a PD-1 agonist.
3. The polypeptide of embodiment 1, wherein the first and second binding domain has a lower affinity for PD-1 as compared to the third and fourth binding domain that binds to PD-1.
4. The polypeptide of embodiment 1, wherein the first and second binding domain has a higher affinity for PD-1 as compared to the third and fourth binding that binds to PD-1.
5. The polypeptide of anyone of embodiments 1-4, wherein the first, second, third, and fourth binding domains are antibodies or antibody fragments that bind to PD-1.
6. The polypeptide of embodiment 5, wherein the first and second binding domain antibody is in the Fab format and the third and fourth binding domain is in a scFv format.
7. The polypeptide of embodiment 6, wherein the antibodies in the Fab format have a higher affinity for PD-1 as compared to the antibodies in the scFv format.
8. The polypeptide of embodiment 6, wherein the antibodies in the Fab format have a lower affinity for PD-1 as compared to the antibodies in the scFv format.
9. The polypeptide of any one of the preceding embodiments, wherein the polypeptide comprises a sequence or antibody as provided for herein, such as the polypeptide chains or fragments of PD1AB4, PD1AB25, PD1AB30, PD1AB53, or PD1AB37.
10. A polypeptide comprising first and second binding domains that bind to PD-1, wherein the first and second binding domains comprise a sequence as set forth herein, such as in PD-1 Antibody Table 4, PD-1 Antibody Table 5, PD-1 Antibody Table 8, PD-1 Antibody Table 9, PD-1 Antibody Table 10, or PD-1 Antibody Table 11 as provided for herein.
11. The polypeptide of embodiment 1, wherein the polypeptide comprises a third and fourth binding domain that bind to PD-1, wherein the third binding domain binds to the same epitope as the first binding domain and the fourth binding domain binds to the same epitope as the second binding domain.
12. The polypeptide of embodiment 11, wherein the first and third binding domains comprise a polypeptide having the same sequence.
13. The polypeptide of embodiment 11 and 12, wherein the second and fourth binding domains comprise a polypeptide having the same sequence.
14. The polypeptide of any one of embodiments 11-13, wherein the first and second binding domains comprise different polypeptide sequences.
15. The polypeptide of embodiment 10, wherein the first and second binding domains are antibodies that bind to PD-1.
16. The polypeptide of embodiments 10 and, wherein the first and second binding domains have identical sequences.
17. The polypeptide of embodiments 10 and 15, wherein the first and second binding domains have different sequences.
18. The polypeptide of embodiment 15-16, wherein the first and second binding domain binds to the same epitope.
19. The polypeptide of embodiment 15-16, wherein the first and second binding domain binds to different epitopes.
20. The polypeptide of any one of embodiments 15-16, wherein one of the first binding domain and the second binding domain is a Fab antibody and the other is a scFv antibody.
21. The polypeptide of any one of embodiments 15-20, wherein the first and second binding domains are linked by a linker.
22. The polypeptide of embodiment 21, wherein the linker comprises an immunoglobulin constant region, such as IgG1, IgG2, IgG3, or IgG4 constant region.
23. The polypeptide of embodiment 21, wherein the linker comprises an immunoglobulin constant region of IgG1.
24. The polypeptide of embodiments 21-23, wherein the linker further comprises a glycine/serine, glycine/alanine linker, glycine/glutamic acid/serine, or alanine/glutamic acid/lysine.
25. The polypeptide of embodiment 24, wherein the glycine/serine linker comprises a sequence of (GGGGS)n, (GGGSE)n or (GGGGA)n, or a combination thereof, wherein each n is independently, 1-4.
26. The polypeptide of embodiment 1 and 10, wherein the polypeptide comprises a first polypeptide chain comprising a Fab heavy chain domain linked to a scFv antibody and a second polypeptide chain comprising a Fab light (kappa) chain domain, wherein the Fab heavy and light chains bind to PD-1 and the scFv antibody binds to PD-1 at the same or different epitopes.
27. The polypeptide of embodiment 26, wherein the scFv comprises heavy chain variable domain linked to a light chain variable domain with a scFv linker.
28. The polypeptide of embodiment 27, wherein the scFV linker comprises a sequence of (GGGGS)n, (GGGSE)n, or (GGGGA)n, or a combination thereof, wherein each n is independently, 1-4.
29. The polypeptide of any one of embodiments 1-22, wherein the first binding domain comprises a sequence as provided in PD-1 Antibody Table 4, PD-1 Antibody Table 5, PD-1 Antibody Table 8, PD-1 Antibody Table 9, PD-1 Antibody Table 10, or PD-1 Antibody Table 11 and the second binding domain comprises a sequence as provided in PD-1 Antibody Table 4, PD-1 Antibody Table 5, PD-1 Antibody Table 8, PD-1 Antibody Table 9, PD-1 Antibody Table 10, or PD-1 Antibody Table 11.
30. The polypeptide of embodiment any one of embodiments 1-28, wherein the first binding domain and the second binding domain are, independently selected, an antibody, or antigen binding fragment thereof, as provided herein.
31. The polypeptide of embodiment 30, wherein the first binding domain and the second binding domain are, independently selected, an antibody, or antigen binding fragment thereof, comprises:
(i) a heavy chain variable region comprising heavy chain CDR1, CDR2, and CDR3 sequences, wherein the heavy chain CDR1 sequence has the amino acid sequence of any of the CDR1 sequences set forth in the PD-1 Antibody Table 4, PD-1 Antibody Table 5, PD-1 Antibody Fab Table 6, or PD-1 Antibody scFv Table 7; the heavy chain CDR2 has the amino acid sequence of any of the CDR2 sequences set forth in the PD-1 Antibody Table 4, PD-1 Antibody Table 5, PD-1 Antibody Fab Table 6, or PD-1 Antibody scFv Table 7, and the heavy chain CDR3 has the amino acid sequence of any of the CDR3 sequences set forth in the PD-1 Antibody Table 4, PD-1 Antibody Table 5, PD-1 Antibody Fab Table 6, or PD-1 Antibody scFv Table 7, or variants of any of the foregoing; and
(ii) a light chain variable region comprising light chain CDR1, CDR2, and CDR3 sequences, wherein the light chain CDR1 sequence has the amino acid sequence of any of the LCDR1 sequences set forth in the PD-1 Antibody Table 4, PD-1 Antibody Table 5, PD-1 Antibody Fab Table 6, or PD-1 Antibody scFv Table 7; the light chain LCDR2 has the amino acid sequence of any of the LCDR2 sequences set forth in the PD-1 Antibody Table 4, PD-1 Antibody Table 5, PD-1 Antibody Fab Table 6, or PD-1 Antibody scFv Table 7, and the light chain CDR3 has the amino acid sequence of any of the LCDR3 sequences set forth in PD-1 Antibody Table 4, PD-1 Antibody Table 5, PD-1 Antibody Fab Table 6, or PD-1 Antibody scFv Table 7, or variants of any of the foregoing.
32. The polypeptide of embodiment 30, wherein the first binding domain and the second binding domain are, independently selected, an antibody, or antigen binding fragment thereof, wherein the antibody, or antigen binding fragment thereof comprises a VK sequence as shown in the PD-1 Antibody Table 4, PD-1 Antibody Table 8, PD-1 Antibody Table 9, PD-1 Antibody Table 10, or PD-1 Antibody Table 11.
33. The polypeptide of embodiment 30, wherein the first binding domain and the second binding domain are, independently selected, an antibody, or antigen binding fragment thereof, wherein the antibody, or antigen binding fragment thereof comprises a VH sequence as shown in the PD-1 Antibody Table 4, PD-1 Antibody Table 8, PD-1 Antibody Table 9, PD-1 Antibody Table 10, or PD-1 Antibody Table 11.
34. The polypeptide of embodiment 30, wherein the first binding domain and the second binding domain are, independently selected, an antibody, or antigen binding fragment thereof, wherein the antibody, or antigen binding fragment thereof comprises a VK sequence as shown in the PD-1 Antibody Tables provided for herein and a VH sequence as shown in the PD-1 Antibody Tables provided for herein.
35. The polypeptide of embodiment 30, wherein the first binding domain and the second binding domain are, independently selected, an antibody, or antigen binding fragment thereof, wherein the antibody, or antigen binding fragment thereof comprises, independently, comprises a heavy chain variable region of Clone ID: PD1AB4 (SEQ ID NO: 35), PD1AB30 (SEQ ID NO: 185), PD1AB17 (SEQ ID NO: 113), PD1AB18 (SEQ ID NO: 120), PD1AB20 (SEQ ID NO: 135), PD1AB25 (SEQ ID NO: 169) of PD-1 Antibody Table 4; PD1AB37 Fab (SEQ ID NO: 256), PD1AB37 scFv (SEQ ID NO: 257) of PD-1 Antibody Table 8; and PD1AB53 Fab (SEQ ID NO: 260), PD1AB53 scFv (SEQ ID NO: 261) of PD-1 Antibody Table 9.
36. The polypeptide of embodiment 30, wherein the first binding domain and the second binding domain are, independently selected, an antibody, or antigen binding fragment thereof, wherein the antibody, or antigen binding fragment thereof comprises, independently, the CDRs of the heavy chain domain of PD1AB4, PD1AB30, PD1AB17, PD1AB18, PD1AB20, PD1AB25 of PD-1 Antibody Table 4; PD1AB37, or PD1AB53 of PD-1 Antibody Fab Table 6 and PD-1 Antibody scFv Table 7.
37. The polypeptide of embodiment 30, wherein the first binding domain and the second binding domain are, independently selected, an antibody, or antigen binding fragment thereof, wherein the antibody, or antigen binding fragment thereof comprises, independently, a heavy chain comprising a first CDR of SEQ ID NO: 37, 171, 115, 122, 137, 171, 267, 238, 279; a second CDR of SEQ ID NO: 38, 172, 116, 123, 138, 172, 229, 239; and a third CDR of SEQ ID NO: 39, 173, 117, 124, 139, 173, 230, 240.
38. The polypeptide of embodiment 30, wherein the first binding domain and the second binding domain are, independently selected, an antibody, or antigen binding fragment thereof, wherein the antibody, or antigen binding fragment thereof comprises, independently, a heavy chain comprising:
a first CDR of SEQ ID NO: 37, a second CDR of SEQ ID NO: 38, and a third CDR of SEQ ID NO: 39;
a first CDR of SEQ ID NO: 171, a second CDR of SEQ ID NO: 172, and a third CDR of SEQ ID NO: 173;
   a first CDR of SEQ ID NO: 115, a second CDR of SEQ ID NO: 116, and a third CDR of SEQ ID NO: 117;
a first CDR of SEQ ID NO: 122, a second CDR of SEQ ID NO: 123, and a third CDR of SEQ ID NO: 124;
   a first CDR of SEQ ID NO: 137, a second CDR of SEQ ID NO: 138, and a third CDR of SEQ ID NO: 139;
   a first CDR of SEQ ID NO: 267, a second CDR of SEQ ID NO: 229, and a third CDR of SEQ ID NO: 230;
   a first CDR of SEQ ID NO: 238, a second CDR of SEQ ID NO: 239, and a third CDR of SEQ ID NO: 240;
   a first CDR of SEQ ID NO: 279, a second CDR of SEQ ID NO: 239, and a third CDR of SEQ ID NO: 240; or
   a first CDR of SEQ ID NO: 267, a second CDR of SEQ ID NO: 229, and a third CDR of SEQ ID NO: 230.
   39. The polypeptide of embodiments 30 and 35-38, wherein the first binding domain and the second binding domain are, independently selected, an antibody, or antigen binding fragment thereof, wherein the antibody, or antigen binding fragment thereof comprises, independently, a light chain variable region of Clone ID: PD1AB4 (SEQ ID NO: 36), PD1AB30 (SEQ ID NO: 170), PD1AB17 (SEQ ID NO: 114), PD1AB18 (SEQ ID NO: 121), PD1AB20 (SEQ ID NO: 136), PD1AB25 (SEQ ID NO: 170) of PD-1 Antibody Table 4; PD1AB37 Fab (SEQ ID NO: 259), PD1AB37 scFv (SEQ ID NO: 258) of PD-1 Antibody Table 8; and PD1AB53 Fab (SEQ ID NO: 263), PD1AB53 scFv (SEQ ID NO: 262) of PD-1 Antibody Table 9.
   40. The polypeptide of embodiments 30 and 35-38, wherein the first binding domain and the second binding domain are, independently selected, an antibody, or antigen binding fragment thereof, wherein the antibody, or antigen binding fragment thereof comprises, independently, a light chain variable region comprising the CDRs of the light chain domain of PD1AB4, PD1AB30, PD1AB17, PD1AB18, PD1AB20, or PD1AB25 of PD-1 Antibody Table 4; PD1AB37, or PD1AB53 of PD-1 Antibody Fab Table 6 and PD-1 Antibody scFv Table 7.
   41. The polypeptide of embodiments 30 and 35-38, wherein the first binding domain and the second binding domain are, independently selected, an antibody, or antigen binding fragment thereof, wherein the antibody, or antigen binding fragment thereof comprises, independently, a light chain variable region comprising a first CDR of SEQ ID NO: 40, 118, 125, 140, 174, 276; a second CDR of SEQ ID NO: 19, 126, 47, 175, 227, 237, and a third CDR of SEQ ID NO: 41, 119, 127, 141, 176.
   42. The polypeptide of embodiments 30 and 35-38, wherein the first binding domain and the second binding domain are, independently selected, an antibody, or antigen binding fragment thereof, wherein the antibody, or antigen binding fragment thereof comprises, independently, a light chain variable region comprising:
a first CDR of SEQ ID NO: 40, a second CDR of SEQ ID NO: 19, and a third CDR of SEQ ID NO: 41;
a first CDR of SEQ ID NO: 118, a second CDR of SEQ ID NO: 19, and a third CDR of SEQ ID NO: 119;
a first CDR of SEQ ID NO: 125, a second CDR of SEQ ID NO: 126, and a third CDR of SEQ ID NO: 127;
a first CDR of SEQ ID NO: 140, a second CDR of SEQ ID NO: 47, and a third CDR of SEQ ID NO: 141;
   a first CDR of SEQ ID NO: 174, a second CDR of SEQ ID NO: 175, and a third CDR of SEQ ID NO: 176;
   a first CDR of SEQ ID NO: 174, a second CDR of SEQ ID NO: 227, and a third CDR of SEQ ID NO: 176;
   a first CDR of SEQ ID NO: 40, a second CDR of SEQ ID NO: 237, and a third CDR of SEQ ID NO: 41;
   a first CDR of SEQ ID NO: 276, a second CDR of SEQ ID NO: 237, and a third CDR of SEQ ID NO: 41; or
   a first CDR of SEQ ID NO: 174, a second CDR of SEQ ID NO: 227, and a third CDR of SEQ ID NO: 176.
   43. The polypeptide of embodiments 30 and 35-38, wherein the first binding domain and the second binding domain are, independently selected, an antibody, or antigen binding fragment thereof, wherein the antibody, or antigen binding fragment thereof comprises, independently: a heavy chain variable region comprising a first CDR of SEQ ID NO: 37, a second CDR of SEQ ID NO: 38, and a third CDR of SEQ ID NO: 39 and a light chain variable region comprising a first CDR of SEQ ID NO: 40, a second CDR of SEQ ID NO: 19, and a third CDR of SEQ ID NO: 41;
   a heavy chain variable region comprising a first CDR of SEQ ID NO: 171, a second CDR of SEQ ID NO: 172, and a third CDR of SEQ ID NO: 173 and a light chain variable region comprising a first CDR of SEQ ID NO: 174, a second CDR of SEQ ID NO: 175, and a third CDR of SEQ ID NO: 176;
   a heavy chain variable region comprising a first CDR of SEQ ID NO: 115, a second CDR of SEQ ID NO: 116, and a third CDR of SEQ ID NO: 117 and a light chain variable region comprising a first CDR of SEQ ID NO: 118, a second CDR of SEQ ID NO: 19, and a third CDR of SEQ ID NO: 119;
   a heavy chain variable region comprising a first CDR of SEQ ID NO: 122, a second CDR of SEQ ID NO: 123, and a third CDR of SEQ ID NO: 124 and a light chain variable region comprising a first CDR of SEQ ID NO: 125, a second CDR of SEQ ID NO: 126, and a third CDR of SEQ ID NO: 127;
   a heavy chain variable region comprising a first CDR of SEQ ID NO: 137, a second CDR of SEQ ID NO: 138, and a third CDR of SEQ ID NO: 139 and a light chain variable region comprising a first CDR of SEQ ID NO: 140, a second CDR of SEQ ID NO: 47, and a third CDR of SEQ ID NO: 141;
   a heavy chain variable region comprising a first CDR of SEQ ID NO: 171, a second CDR of SEQ ID NO: 172, and a third CDR of SEQ ID NO: 173 and a light chain variable region comprising a first CDR of SEQ ID NO: 174, a second CDR of SEQ ID NO: 175, and a third CDR of SEQ ID NO: 176;
   a heavy chain variable region comprising a first CDR of SEQ ID NO: 267, a second CDR of SEQ ID NO: 229, and a third CDR of SEQ ID NO: 230 and a light chain variable region comprising a first CDR of SEQ ID NO: 174, a second CDR of SEQ ID NO: 227, and a third CDR of SEQ ID NO: 176;
   a heavy chain variable region comprising a first CDR of SEQ ID NO: 238, a second CDR of SEQ ID NO: 239, and a third CDR of SEQ ID NO: 240 and a light chain variable region comprising a first CDR of SEQ ID NO: 40, a second CDR of SEQ ID NO: 237, and a third CDR of SEQ ID NO: 41;
   a heavy chain variable region comprising a first CDR of SEQ ID NO: 279, a second CDR of SEQ ID NO: 239, and a third CDR of SEQ ID NO: 240 and a light chain variable region comprising a first CDR of SEQ ID NO: 276, a second CDR of SEQ ID NO: 237, and a third CDR of SEQ ID NO: 41; or
   a heavy chain variable region comprising a first CDR of SEQ ID NO: 267, a second CDR of SEQ ID NO: 229, and a third CDR of SEQ ID NO: 230 and a light chain variable region comprising a first CDR of SEQ ID NO: 174, a second CDR of SEQ ID NO: 227, and a third CDR of SEQ ID NO: 176.
   44. The polypeptide of embodiments 30 and 35-38, wherein the first binding domain and the second binding domain are, independently selected, an antibody, or antigen binding fragment thereof, wherein the antibody, or antigen binding fragment thereof comprises, independently, a heavy chain comprising a sequence of SEQ ID NO: 35, SEQ ID NO: 185, SEQ ID NO: 113, SEQ ID NO: 120, SEQ ID NO: 135, SEQ ID NO: 169, SEQ ID NO: 256, SEQ ID NO: 257, SEQ ID NO: 260, or SEQ ID NO: 261, and a light chain variable region comprising a sequence of SEQ ID NO: 36, SEQ ID NO: 170, SEQ ID NO: 114, SEQ ID NO: 121, SEQ ID NO: 136, SEQ ID NO: 170, SEQ ID NO: 259, SEQ ID NO: 258, SEQ ID NO: 263, or SEQ ID NO: 262.
   45. The polypeptide of embodiments 30 and 35-38, wherein the first binding domain and the second binding domain are, independently selected, an antibody, or antigen binding fragment thereof, wherein the antibody, or antigen binding fragment thereof comprises, independently: a heavy chain variable region comprising a sequence of SEQ ID NO: 35 and a light chain variable region comprising a sequence of SEQ ID NO: 36;
   a heavy chain variable region comprising a sequence of SEQ ID NO: 113 and a light chain variable region comprising a sequence of SEQ ID NO: 114;
   a heavy chain variable region comprising a sequence of SEQ ID NO: 120 and a light chain variable region comprising a sequence of SEQ ID NO: 121;
   a heavy chain variable region comprising a sequence of SEQ ID NO: 135 and a light chain variable region comprising a sequence of SEQ ID NO: 136;
   a heavy chain variable region comprising a sequence of SEQ ID NO: 169 and a light chain variable region comprising a sequence of SEQ ID NO: 170;
   a heavy chain variable region comprising a sequence of SEQ ID NO: 185 and a light chain variable region comprising a sequence of SEQ ID NO: 170;
   a heavy chain variable region comprising a sequence of SEQ ID NO: 256 and a light chain variable region comprising a sequence of SEQ ID NO: 259;
   a heavy chain variable region comprising a sequence of SEQ ID NO: 257 and a light chain variable region comprising a sequence of SEQ ID NO: 258;
   a heavy chain variable region comprising a sequence of SEQ ID NO: 260 and a light chain variable region comprising a sequence of SEQ ID NO: 263; or
   a heavy chain variable region comprising a sequence of SEQ ID NO: 261 and a light chain variable region comprising a sequence of SEQ ID NO: 262.
   46. The polypeptide of embodiment 1 and 10, wherein the polypeptide comprises a first polypeptide chain and a second polypeptide chain wherein:
the first polypeptide chain has a formula of from N-terminus to C-terminus: [VH-A]-[CH1]-[CH2]-[CH3]-[Linker 1]-[VH-B]-[Linker 2]-[VK-B]; or [VH-A]-[CH1]-[CH2]-[CH3]-[Linker 1]-[VK-B]-[Linker 2]-[VH-B];
the second polypeptide chain has a formula of from N-terminus to C-terminus: [VK-A]-[CK],
   wherein:
   VH-A=Variable heavy chain domain of a PD1 antibody as provided herein;
   VK-A=Variable light domain of a PD1 antibody as provided herein
   VH-B=Variable heavy chain domain of a PD1 antibody as provided herein;
   VK-B=Variable light domain of a PD1 antibody as provided herein
   CH1=Constant heavy domain 1 of human IgG1
   CH2=Constant heavy domain 2 of human IgG1
   CH3=Constant heavy domain 3 of human IgG1
   CK=Constant domain of kappa light chain
   Linker 1 is a glycine/serine, glycine/alanine, glycine/glutamic acid/serine, or alanine/glutamic acid/lysine linker
   Linker 2 is a glycine/serine, glycine/alanine, glycine/glutamic acid/serine, or alanine/glutamic acid/lysine linker,
   wherein VH-A, VK-A, VH-B, and VK-B can be from the same antibody or different.
   47. The polypeptide of embodiment 1 and 10, wherein the polypeptide comprises a first polypeptide chain and a second polypeptide chain, wherein:
   the first polypeptide chain has a formula of from N-terminus to C-terminus:

### [VH-B]-[CH1]-[CH2]-[CH3]-[Linker 1]-[VH-A]-[Linker 2]-[VK-A]; or

### [VH-B]-[CH1]-[CH2]-[CH3]-[Linker 1]-[VK-A]-[Linker 2]-[VH-A];

the second polypeptide chain has a formula of from N-terminus to C-terminus:

### [VK-B]-[CK],

wherein:
VH-A=Variable heavy chain domain of a PD1 antibody as provided herein;
VK-A=Variable light domain of a PD1 antibody as provided herein;
VH-B=Variable heavy chain domain of a PD1 antibody as provided herein;
VK-B=Variable light domain of a PD1 antibody as provided herein;
CH1=Constant heavy domain 1 of human IgG1, such as provided herein;
CH2=Constant heavy domain 2 of human IgG1, such as provided herein;
CH3=Constant heavy domain 3 of human IgG1, such as provided herein;
CK=Constant domain of kappa light chain, such as provided herein;
Linker 1 is a glycine/serine, glycine/alanine, glycine/glutamic acid/serine, or alanine/glutamic acid/lysine linker;
Linker 2 is a glycine/serine, glycine/alanine, glycine/glutamic acid/serine, or alanine/glutamic acid/lysine linker;
wherein VH-A, VK-A, VH-B, and VK-B can be from the same antibody or different.
48. The polypeptide of embodiments 46 or 47, wherein VH-A and VH-B comprise, independently:
(i) a heavy chain variable region comprising heavy chain CDR1, CDR2, and CDR3 sequences, wherein the heavy chain CDR1 sequence has the amino acid sequence of any of the CDR1 sequences set forth in the PD-1 Antibody Table 4, PD-1 Antibody Table 5, PD-1 Antibody Fab Table 6, or PD-1 Antibody scFv Table 7; the heavy chain CDR2 has the amino acid sequence of any of the CDR2 sequences set forth in the PD-1 Antibody Table 4, PD-1 Antibody Table 5, PD-1 Antibody Fab Table 6, or PD-1 Antibody scFv Table 7, and the heavy chain CDR3 has the amino acid sequence of any of the CDR3 sequences set forth in the PD-1 Antibody Table 4, PD-1 Antibody Table 5, PD-1 Antibody Fab Table 6, or PD-1 Antibody scFv Table 7, or variants of any of the foregoing.
49. The polypeptide of embodiment 48, wherein VH-A and VH-B are different.
50. The polypeptide of any one of embodiments 46-49, wherein VH-A comprises a heavy chain variable region comprising a first CDR of SEQ ID NO: 37, a second CDR of SEQ ID NO: 38, and a third CDR of SEQ ID NO: 39.
51. The polypeptide of any one of embodiments 46-49, wherein VH-B comprises a heavy chain variable region comprising a first CDR of SEQ ID NO: 37, a second CDR of SEQ ID NO: 38, and a third CDR of SEQ ID NO: 39.
52. The polypeptide of 46, wherein VH-A comprises a heavy chain variable region comprising a first CDR of SEQ ID NO: 37, a second CDR of SEQ ID NO: 38, and a third CDR of SEQ ID NO: 39 and VH-B comprises a heavy chain variable region comprising a first CDR of SEQ ID NO: 37, a second CDR of SEQ ID NO: 38, and a third CDR of SEQ ID NO: 39.
53. The polypeptide of any one of embodiments 46-49, wherein VH-A comprises a heavy chain variable region comprising a first CDR of SEQ ID NO: 171, a second CDR of SEQ ID NO: 172, and a third CDR of SEQ ID NO: 173.
54. The polypeptide of any one of embodiments 46-49, wherein VH-B comprises a heavy chain variable region comprising a first CDR of SEQ ID NO: 171, a second CDR of SEQ ID NO: 172, and a third CDR of SEQ ID NO: 173.
55. The polypeptide of any one of embodiments 46-49, wherein VH-A comprises a heavy chain variable region comprising a first CDR of SEQ ID NO: 267, a second CDR of SEQ ID NO: 229, and a third CDR of SEQ ID NO: 230.
56. The polypeptide of any one of embodiments 46-49, wherein VH-B comprises a heavy chain variable region comprising a first CDR of SEQ ID NO: 267, a second CDR of SEQ ID NO: 229, and a third CDR of SEQ ID NO: 230.
57. The polypeptide of any one of embodiments 46-49, wherein VH-A comprises a heavy chain variable region comprising a first CDR of SEQ ID NO: 238, a second CDR of SEQ ID NO: 239, and a third CDR of SEQ ID NO: 240.
58. The polypeptide of any one of embodiments 46-49, wherein VH-B comprises a heavy 25 chain variable region comprising a first CDR of SEQ ID NO: 238, a second CDR of SEQ ID NO: 239, and a third CDR of SEQ ID NO: 240.
59. The polypeptide of any one of embodiments 46-49, wherein VH-A comprises a heavy chain variable region comprising a first CDR of SEQ ID NO: 279, a second CDR 30 of SEQ ID NO: 239, and a third CDR of SEQ ID NO: 240.
60. The polypeptide of any one of embodiments 46-49, wherein VH-B comprises a heavy chain variable region comprising a first CDR of SEQ ID NO: 279, a second CDR of SEQ ID NO: 239, and a third CDR of SEQ ID NO: 240.
61. The polypeptide of any one of embodiments 46-49, wherein VH-A comprises a heavy chain variable region comprising a first CDR of SEQ ID NO: 267, a second CDR of SEQ ID NO: 229, and a third CDR of SEQ ID NO: 230.
62. The polypeptide of any one of embodiments 46-49, wherein VH-B comprises a heavy chain variable region comprising a first CDR of SEQ ID NO: 267, a second CDR of SEQ ID NO: 229, and a third CDR of SEQ ID NO: 230.
63. The polypeptide of 46-49, wherein VH-A comprises a heavy chain variable region comprising a first CDR of SEQ ID NO: 279, a second CDR of SEQ ID NO: 239, and a third CDR of SEQ ID NO: 240 and VH-B comprises a heavy chain variable region comprising a first CDR of SEQ ID NO: 267, a second CDR of SEQ ID NO: 229, and a third CDR of SEQ ID NO: 230.
64. The polypeptide of 46-49, wherein VH-A comprises a heavy chain variable region comprising a first CDR of SEQ ID NO: 267, a second CDR of SEQ ID NO: 229, and a third CDR of SEQ ID NO: 230 and VH-B comprises a heavy chain variable region comprising a first CDR of SEQ ID NO: 238, a second CDR of SEQ ID NO: 239, and a third CDR of SEQ ID NO: 240.
65. The polypeptide of any one of embodiments 46-63, wherein VK-A and VK-B, each, independently, comprise a light chain variable region comprising light chain CDR1, CDR2, and CDR3 sequences, wherein the light chain CDR1 sequence has the amino acid sequence of any of the LCDR1 sequences set forth in the PD-1 Antibody Table 4, PD-1 Antibody Table 5, PD-1 Antibody Fab Table 6, or PD-1 Antibody scFv Table 7; the light chain LCDR2 has the amino acid sequence of any of the LCDR2 sequences set forth in the PD-1 Antibody Table 4, PD-1 Antibody Table 5, PD-1 Antibody Fab Table 6, or PD-1 Antibody scFv Table 7, and the light chain CDR3 has the amino acid sequence of any of the LCDR3 sequences set forth in PD-1 Antibody Table 4, PD-1 Antibody Table 5, PD-1 Antibody Fab Table 6, or PD-1 Antibody scFv Table 7, or variants of any of the foregoing.
66. The polypeptide of any one of embodiments 46-65, wherein VK-A or VK-B comprises a light chain variable region comprising a first CDR of SEQ ID NO: 40, a second CDR of SEQ ID NO: 19, and a third CDR of SEQ ID NO: 41.
67. The polypeptide of any one of embodiments 46-65, wherein VK-A or VK-B comprises a light chain variable region comprising a first CDR of SEQ ID NO: 174, a second CDR of SEQ ID NO: 175, and a third CDR of SEQ ID NO: 176.
68. The polypeptide of any one of embodiments 46-65, wherein VK-A or VK-B comprises a light chain variable region comprising a first CDR of SEQ ID NO: 174, a second CDR of SEQ ID NO: 227, and a third CDR of SEQ ID NO: 176.
69. The polypeptide of any one of embodiments 46-65, wherein VK-A or VK-B comprises a light chain variable region comprising a first CDR of SEQ ID NO: 40, a 20 second CDR of SEQ ID NO: 237, and a third CDR of SEQ ID NO: 41.
70. The polypeptide of any one of embodiments 46-65, wherein VK-A or VK-B comprises a light chain variable region comprising a first CDR of SEQ ID NO: 276, a second CDR of SEQ ID NO: 237, and a third CDR of SEQ ID NO: 41.
71. The polypeptide of any one of embodiments 46-65, wherein VK-A or VK-B comprises a light chain variable region comprising a first CDR of SEQ ID NO: 174, a second CDR of SEQ ID NO: 227, and a third CDR of SEQ ID NO: 176.
72. The polypeptide of embodiments 46 or 47, wherein:
VH-A comprises a heavy chain variable region comprising a first CDR of SEQ ID NO: 37, a second CDR of SEQ ID NO: 38, and a third CDR of SEQ ID NO: 39;
VH-B comprises a heavy chain variable region comprising a first CDR of SEQ ID NO: 171, a second CDR of SEQ ID NO: 172, and a third CDR of SEQ ID NO: 173;
VK-A comprises a light chain variable region comprising a first CDR of SEQ ID NO: 40, a second CDR of SEQ ID NO: 19, and a third CDR of SEQ ID NO: 41;
VK-B comprises a light chain variable region comprising a first CDR of SEQ ID NO: 174, a second CDR of SEQ ID NO: 175, and a third CDR of SEQ ID NO: 176;
VK-A comprises a light chain variable region comprising a first CDR of SEQ ID NO: 276, a second CDR of SEQ ID NO: 237, and a third CDR of SEQ ID NO: 41;
VK-B comprises a light chain variable region comprising a first CDR of SEQ ID NO: 174, a second CDR of SEQ ID NO: 227, and a third CDR of SEQ ID NO: 176; or
VK-A comprises a light chain variable region comprising a first CDR of SEQ ID NO: 174, a second CDR of SEQ ID NO: 227, and a third CDR of SEQ ID NO: 176; VK-B comprises a light chain variable region comprising a first CDR of SEQ ID NO: 40, a second CDR of SEQ ID NO: 237, and a third CDR of SEQ ID NO: 41.
   73. The polypeptide of any one of embodiments 46-72, wherein Linker 1 is (GGGGS)n, wherein n is 1-4.
   74. The polypeptide of any one of embodiments 46-72, wherein Linker 2 is (GGGGS)n, wherein n is 1-4.
   75. A biparatopic polypeptide, comprising a first, second, third, and fourth binding domain, wherein two of the a first, second, third, and fourth binding domains bind to a first epitope on PD-1 and the other two of first, second, third, and fourth binding domains bind to a second epitope on PD-1, wherein the first and second epitopes are different.
   76. The biparatopic polypeptide of embodiment 75, wherein the first and second epitopes are non-overlapping.
   77. The biparatopic polypeptide of embodiments 75 or 76, wherein the first, second, third, and fourth binding domains comprise a PD-1 antibody, such as those provided herein.
   78. The biparatopic polypeptide of any one of embodiments 75-77, wherein two of first, second, third, and fourth binding domains comprise CDRs from one of PD1AB4, PD1AB30, PD1AB17, PD1AB18, PD1AB20, PD1AB25, PD1AB37, or PD1AB53, and the other two comprise CDRs from one of PD1AB4, PD1AB30, PD1AB17, PD1AB18, PD1AB20, PD1AB25, PD1AB37, or PD1AB53, wherein not all of the binding domains are the same.
   79. A pharmaceutical composition comprising a polypeptide, protein, or an antibody of any one of embodiments 1-78.
   80. A method of treating Systemic Lupus Erythematosus (SLE), Aicardi-Goutieres syndrome, bilateral striatal necrosis, chronic atypical neutrophilic dermatosis with lipodystrophy and elevated temperature (CANDLE), complete non-penetrance, dyschromatosis symmetrica hereditaria, familial chilblain lupus, Japanese autoinflammatory syndrome with lipodystrophy (JASL), joint contractures, muscle atrophy, microcytic anaemia, panniculitis, and lipodystrophy (JMP), Mendelian susceptibility to mycobacterial disease (MSMD), Nakajo-Nishimura syndrome, retinal vasculopathy with cerebral leukodystrophy (RVCL), spastic paraparesis, STING-associated vasculopathy with onset in infancy (SAVI), Singleton-Merten syndrome, or spondylochondromatosis (SPENCD) in a subject, the method comprising administering to the subject a polypeptide of any one of embodiments 1-65 or the composition of embodiment 66.
   81. A method of treating a subject with inflammatory bowel disease, the method comprising administering a polypeptide, protein or antibody of any one of embodiments 1-65 or the pharmaceutical composition of embodiment 79 to the subject to treat the inflammatory bowel disease.
   82. The method of embodiment 81, wherein the subject with inflammatory bowel disease has Crohn's disease.
   83. The method of embodiment 81, wherein the subject with inflammatory bowel disease has ulcerative colitis.
   84. A method of treating a subject with autoimmune hepatitis, the method comprising administering a polypeptide, protein or antibody of any one of embodiments 1-78 or the pharmaceutical composition of embodiment 79 to the subject to treat the autoimmune hepatitis.
   85. A method of treating primary sclerosing cholangitis the method comprising administering a polypeptide, protein or antibody of any one of embodiments 1-78 or the pharmaceutical composition of embodiment 79 to the subject to treat the primary sclerosing cholangitis.
   86. A method of treating Type 1 diabetes the method comprising administering a polypeptide, protein or antibody of any one of embodiments 1-78 or the pharmaceutical composition of embodiment 79 to the subject to treat the Type 1 diabetes.
   87. A method of treating a transplant subject comprising administering a therapeutically effective amount of a polypeptide, protein or antibody of any one of embodiments 1-78 or the pharmaceutical composition of embodiment 79 to the subject, thereby treating a transplant (recipient) subject.
   88. A method of treating GVHD in a subject having a transplanted a donor tissue comprising administering a therapeutically effective amount of a polypeptide, protein or antibody of any one of embodiments 1-78 or the pharmaceutical composition of embodiment 79 to the subject.
   89. A method of treating a subject having, or at risk, or elevated risk, for having, an autoimmune disorder, comprising administering a therapeutically effective amount of a 30 polypeptide, protein or antibody of any one of embodiments 1-78 or the pharmaceutical composition of embodiment 79, thereby treating the subject.
   90. A method of agonizing the activity of PD-1 in a subject, the method comprising administering a therapeutically effective amount of a polypeptide, protein or antibody of any one of embodiments 1-78 or the pharmaceutical composition of embodiment 79, thereby treating the subject.
   91. A method of treating an interferonopathy in a subject, the method comprising administering to the subject a polypeptide, protein or antibody of any one of embodiments 1-78 or the pharmaceutical composition of embodiment 79, thereby treating the subject.
   92. The method of embodiment 91, wherein the interferonopathy is a Type I interferonopathy.
   93. The method of embodiments 91 or 92, wherein the subject is a subject in need thereof.
   94. The method of any one of embodiments 91-93, wherein the Type I interferonopathy is Systemic Lupus Erythematosus (SLE), Aicardi-Goutieres syndrome, bilateral striatal necrosis, chronic atypical neutrophilic dermatosis with lipodystrophy and elevated temperature (CANDLE), complete non-penetrance, dyschromatosis symmetrica hereditaria, familial chilblain lupus, Japanese autoinflammatory syndrome with lipodystrophy (JASL), joint contractures, muscle atrophy, microcytic anaemia, panniculitis, and lipodystrophy (JMP), Mendelian susceptibility to mycobacterial disease (MSMD), Nakajo-Nishimura syndrome, retinal vasculopathy with cerebral leukodystrophy (RVCL), spastic paraparesis, STING-associated vasculopathy with onset in infancy (SAVI), Singleton-Merten syndrome, or spondylochondromatosis (SPENCD).
   95. A method of inhibiting the production of interferon from plasmacytoid dendritic cells, the method comprising administering to the subject a polypeptide, protein or antibody of any one of embodiments 1-78 or the pharmaceutical composition of embodiment 79.
   96. The method of embodiment 95, wherein the plasmacytoid dendritic cells are activated plasmacytoid dendritic cells.
   97. The method of embodiments 95 or 96, wherein the activated plasmacytoid dendritic cells is TLR9 mediated activation.
   98. The method of any one of embodiments 95-97, wherein the production of interferon is reduced by about, or at least, 10, 20, 30, 40, 50, 60, 70, 80, 90, or 95% as compared to the amount of interferon produced in the absence of the polypeptide, protein or antibody of any one of embodiments 1-78 or the pharmaceutical composition of embodiment 79.
   99. A method of treating a TLR9 mediated disorder, the method comprising the method comprising administering to the subject a polypeptide, protein or antibody of any one of embodiments 1-77 or the pharmaceutical composition of embodiment 78.
   100. The method of embodiment 99, wherein the TLR9 mediated disorder is Systemic Lupus Erythematosus (SLE), Aicardi-Goutieres syndrome, bilateral striatal necrosis, chronic atypical neutrophilic dermatosis with lipodystrophy and elevated temperature (CANDLE), complete non-penetrance, dyschromatosis symmetrica hereditaria, familial chilblain lupus, Japanese autoinflammatory syndrome with lipodystrophy (JASL), joint contractures, muscle atrophy, microcytic anaemia, panniculitis, and lipodystrophy (JMP), Mendelian susceptibility to mycobacterial disease (MSMD), Nakajo-Nishimura syndrome, retinal vasculopathy with cerebral leukodystrophy (RVCL), spastic paraparesis, STING-associated vasculopathy with onset in infancy (SAVI), Singleton-Merten syndrome, or spondylochondromatosis (SPENCD).
   101. A method of inhibiting the expression of OAS1, IFIT3, MX1 and IFN-β1 in a cell or a subject, the method comprising administering to the subject or contacting a cell with a polypeptide, protein or antibody of any one of embodiments 1-78 or the pharmaceutical composition of embodiment 79.
   102. The method of embodiment 101, wherein the expression of OAS1, IFIT3, MX1 and IFN-β1 is the gene expression.
   103. The method of embodiments 101 or 102, wherein the cell is a plasmacytoid dendritic cell.
   104. The method of embodiments 101-103, wherein the plasmacytoid dendritic cell is an activated plasmacytoid dendritic cell.
   105. A nucleic acid encoding the polypeptide, protein or antibody of any of embodiments 1-78.
   106. A vector comprising the nucleic acid of embodiment 105.
   107. A cell comprising the nucleic acid of embodiment 105 or the vector of embodiment 106.
   108. A method of making a polypeptide, protein or antibody of any of embodiments 1-78 comprising culturing a cell of embodiment 107 to make the polypeptide, protein or antibody of any of embodiments 1-78.
   109. A method of making a nucleic acid sequence encoding a polypeptide, protein or antibody of any of embodiments 1-78, comprising
a) providing a vector comprising sequence encoding a targeting moiety and inserting into the vector sequence encoding an effector binding/modulating moiety to form a sequence encoding a therapeutic compound; or
b) providing a vector comprising sequence encoding an effector binding/modulating moiety and inserting into the vector sequence encoding a targeting moiety to form a sequence encoding a therapeutic compound,
thereby making a sequence encoding the polypeptide, protein or antibody of any of embodiments 1-78.

### EXAMPLES

### Example 1. Biparatopic PD-1 Agonist Show Strong Soluble PD-1 Agonism

A biparatopic polypeptide comprising the CDRs of two of PD1AB4, PD1AB30, PD1AB17, PD1AB18, PD1AB20, or PD1AB25 is produced and tested for its ability to agonize PD-1 activity. The format that is made is illustrated in FIG. 2. It is found that the biparatopic molecule has a surprising and unexpected superior ability to agonize PD-1 as compared to a molecule that is not biparatopic.

### Example 2. Biparatopic Molecules Bind Human or Mouse PD-1 by Biosensor Assay

Anti-human IgG Fc (AHC) biosensors were equilibrated in assay buffer for 20 minutes. Test articles were diluted to 10 μg/mL in assay buffer (lx PBS, 1% BSA, 0.05% Tween20). A seven-point serial dilution of human PD-1 was prepared in assay buffer, starting at 1000 nM down to 15.625 nM (human PD-1) or 2000 nM down to 31.25 nM (mouse PD-1). Test articles were loaded on tips for 180 s followed by a 180 s association phase with PD-1 and 180 s dissociation phase in assay buffer. PD1AB43 bound to human PD-1 with Kd (M) of 1.87E-08, KD Error of 3.21E-10, Kon (1/ms) of 6.38E+04, Kon Error of 3.74E+02, Kdis (1/s) of 1.20E-03, Kdis Error of 1.93E-05, and response of 0.1549; and PD1AB53 showed Kd of 6.29E-10, KD Error of 1.64E-10, Kon of 6.76E+04, Kon Error of 2.42E+02, Kdis of 4.26E+02, Kdis Error of 1.11E-05, and response of 0.2273. PD1AB43 bound to mouse PD-1 with Kd (M) of 1.43E-06, KD Error of 5.88E-08, Kon (1/ms) of 2.82E+04, Kon Error of 9.91E+04, Kdis (1/s) of 4.04E-02, Kdis Error of 8.53E-04, and response of 0.0546; and PD1AB53 showed Kd of 3.84E-06, KD Error of 4.27E-07, Kon of 5.94E+03, Kon Error of 6.33E+02, Kdis of 2.28E-02, Kdis Error of 7.00E-04, and response of 0.0367. Biparatopic molecules showed binding to human and mouse PD-1.

### Example 3. Biparatopic Molecules Bind Human, Cyno, or Mouse PD-1 by ELISA

Plates were coated with 2 μg/mL huPD-1 in 1×PBS at 4° C. overnight. Plates were blocked with 1×PBS with 1% BSA. Antibodies were tested in duplicate for binding with a 3-fold, dilution series from 50 nM to 0.05 nM. Antibody binding was detected by Anti-Kappa-HRP antibody, signal is background subtracted for coated wells with 2° antibody only. PD1AB43, PD1AB86, PD1AB87, PD1AB88, PD1AB53, PD1AB69, PD1AB64, and PD1AB76 bound to human, cyno, or mouse PD-1. Biparatopic molecules showed binding to human, cyno, and mouse PD-1.

### Example 4. Biparatopic Molecules Bind to Human, Cyno, or Mouse PD-1 by Biosensor Assay

Anti-human IgG Fc (AHC) biosensors were equilibrated in assay buffer for 20 minutes. Test article was diluted to 10 μg/mL in assay buffer (lx PBS, 1% BSA, 0.05% Tween20). A seven-point serial dilution of human PD-1 was prepared in assay buffer, starting at 1000 nM down to 15.625 nM (human and cyno PD-1) or 2000 nM down to 31.25 nM (mouse PD-1). Test article was loaded on tips for 180 s followed by a 180 s association phase with PD-1 and 180 s dissociation phase in assay buffer. PD1AB53 bound to human PD-1 with Kd (M) of 3.30E-08, Kon (1/ms) of 4.13E+04, and Kdis (1/s) of 1.36E-03; PD1AB64 showed Kd of 2.13E-08, Kon of 3.95E+04, and Kdis of 8.41E-04; PD1AB37 showed Kd of 1.83E-08, Kon of 4.50E+04, and Kdis of 8.24E-04; and PD1AB38 showed Kd of 5.07E-08, Kon of 5.64E+04, and Kdis of 2.86E-03. PD1AB53 bound to cynomolgus PD-1 with Kd (M) of 1.35E-08, Kon (1/ms) of 3.94E+04, and Kdis (1/s) of 5.31E-04; PD1AB64 showed Kd of 1.66E-08, Kon of 3.87E+04, and Kdis of 6.41E-04; PD1AB37 showed Kd of 1.54E-08, Kon of 3.68E+04, and Kdis of 5.66E-04; and PD1AB38 showed Kd of 2.83E-08, Kon of 3.79E+04, and Kdis of 1.08E-03. PD1AB53 bound to mouse PD-1 with Kd (M) of 2.46E-05, Kon (1/ms) of 2.67E+03, and Kdis (1/s) of 6.57E-02; PD1AB64 bound weakly; PD1AB37 showed Kd of 4.86E-06, Kon of 5.05E+03, and Kdis of 2.45E-02; and PD1AB38 showed Kd of 9.79E-07, Kon of 2.34E+04, and Kdis of 2.29E-02. Biparatopic molecules showed binding to human, cyno, and mouse PD-1.

### Example 5. Biparatopic Molecules Bind to Human, Cyno, or Mouse PD-1 with Consistent Affinity Across Species by Biosensor Assay

Anti-human IgG Fc (AHC) or Streptavidin (SA) biosensor were equilibrated in assay buffer for 20 minutes. Fe-tagged mouse and cynomolgus PD-1 was diluted to 5 μg/mL and biotinylated huPD-1 article was diluted to 0.5 μg/mL in assay buffer (1×PBS, 1% BSA, 0.05% Tween20). A seven-point serial dilution of test article Fab and scFv fragments were prepared in assay buffer PD1AB53 Fab (500 nM), PD1AB37 and PD1AB38 Fab (1000 nM); scFv Fragments (2000 nM). PD-1 was loaded on tips for 180 s followed by a 180 s association phase with PD-1 and 180 s dissociation phase in assay buffer. The Fab moiety of the PD1AB37 bound to human PD-1 with Kd of 330 nM, while the scFv moiety bound with Kd of 89.9 nM; the Fab moiety of the PD1AB38 bound to human PD-1 with Kd of 140 nM, while the scFv moiety bound with Kd of 89.9 nM; the Fab moiety of the PD1AB53 bound to human PD-1 with Kd of 32.6 nM, while the scFv moiety bound with Kd of 1.35 uM. The Fab moiety of the PD1AB37 bound to cyno PD-1 with Kd of 497 nM, while the scFv moiety bound with Kd of 58.6 nM; the Fab moiety of the PD1AB38 bound to cyno PD-1 with Kd of 172 nM, while the scFv moiety bound with Kd of 58.6 nM; the Fab moiety of the PD1AB53 bound to cyno PD-1 with Kd of 30.3 nM, while the scFv moiety bound with Kd of 1.17 uM. The Fab moiety of the PD1AB37 bound to mouse PD-1 with Kd of 1.35 uM, while the scFv moiety showed no binding; the Fab moiety of the PD1AB38 bound to mouse PD-1 with Kd of 176 nM, while the scFv moiety showed no binding; the Fab moiety of the PD1AB53 showed no binding to mouse PD-1, while the scFv moiety bound with Kd of 1.06 uM.

### Example 6. Biparatopic Molecules Show Expected Molecular Weight by SEC-MALS

Molecular weight of PD1AB53, PD1AB37, and PD1AB38 was assessed using SEC-MALS according to the standard protocol. Briefly, 20 μL of test article was injected onto Zenix SEC-300 column and eluted for 10 minutes at 0.35 mL/min. PD1AB53 was predicted to have the size of 198.4 kDa, and showed actual molecule weight of 205.7 kDa, comprising 3.4% error, and 0.025 kDa glycan. PD1AB37 was predicted to have the size of 197.7 kDa, and showed actual molecule weight of 188.9 kDa, comprising 4.3% error, and 0.028 kDa glycan. PD1AB38 was predicted to have the size of 197.9 kDa, and showed actual molecule weight of 195.4 kDa, comprising 3.1% error, and 0.049 kDa glycan. Biparatopic molecules closely reflect their expected size and show no unexpected glycosylation.

### Example 7. Biparatopic Molecule, PD1AB53, has Favorable Storage Stability Profile

Test article was concentrated to approximately 30 mg/mL and incubated at 4° C. and 37° C. for 0, 3, 14, 21, and 28 days. Approximately 15 μg of test article was injected onto AdvanceBio SEC-300A column and eluted for 10 minutes at 0.35 mL/min. PD1AB53 was formulated in: a) 25 mM sodium acetate, 100 mM sodium chloride pH 6.0; b) 25 mM sodium acetate, 100 mM sodium chloride, 200 mm sucrose pH 6.0; and c) 25 mM sodium phosphate, 250 mM sodium chloride, pH 7.0. Stability was calculated as % POI and showed 99.2% in a), and 99.3% in b) on day 0; 99.1% in a), 99.3% in b), and 99.1% in c) on day 3; 98.9% in a), 99.2% in b), and 99.0% in c) on day 14; 98.9% in a), 99.0% in b), and 98.7% in c) on day 21; and 98.9% in a), 99.0% in b), and 93.6% in c) on day 28. PD1AB53 has good stability at 4° C. up to 28 days at approximately 23 mg/ml in sodium acetate buffers, and showed aggregation over time at 37° C.

### Example 8. Biparatopic Molecule, PD1AB37, has Favorable Storage Stability Profile

Test article was concentrated to approximately 30 mg/mL and incubated at 4° C. and 37° C. for 0, 3, 14, 21, and 28 days. Approximately 15 μg of test article was injected onto AdvanceBio SEC-300A column and eluted for 10 minutes at 0.35 mL/min. PD1AB37 was formulated in: a) 25 mM sodium phosphate, 250 mM sodium chloride, pH 7.0; b) 25 mM sodium phosphate, 250 mM sodium chloride, 200 mM sucrose pH 7.0; and c) 25 mM sodium phosphate, 250 mM sodium chloride, 200 mM glutamate, pH 7.0. Stability was calculated as % POI and showed 61.9% in a), 63.7% in b), and 62.6% in c) on day 0; 60.1% in a), 61.6% in b), and 62.2% in c) on day 3; 57.2% in a), 64.9% in b), and 62.2% in c) on day 14; 60.5% in a), 65.1% in b), and 62.3% in c) on day 21; and 59.1% in a), 65.2% in b), and 60.8% in c) on day 28. PD1AB37 was stable at 4° C. up to 28 days at approximately 15 mg/ml in sodium acetate buffers, and showed aggregation as a result of concentration. Aggregation and slight degradation over time was evident in the accelerated storage conditions at 37° C.

### Example 9. Biparatopic Molecule, PD1AB38, has Favorable Storage Stability Profile

Test article was concentrated to approximately 30 mg/mL and incubated at 4° C. and 37° C. for 0, 3, 14, 21, and 28 days. Approximately 15 μg of test article was injected onto AdvanceBio SEC-300A column and eluted for 10 minutes at 0.35 mL/min. PD1AB37 was formulated in: a) 25 mM sodium phosphate, 250 mM sodium chloride, pH 7.0; b) 25 mM sodium phosphate, 250 mM sodium chloride, 200 mM sucrose pH 7.0. Stability was calculated as % POI and showed 72.8% in a), and 78.0% in b) on day 0; 70.3% in a), and 77.1% in b) on day 3; 67.6% in a), and 77.1% in b) on day 14; 66.6% in a), and 77.2% in b) on day 21; and 71.9% in a), and 76.9% in b) on day 28. PD1AB38 was stable at 4° C. up to 28 days at approximately 15 mg/ml in sodium acetate buffers, and showed aggregation as a result of concentration. Aggregation and slight degradation over time was evident in the accelerated storage conditions at 37° C.

### Example 10. Biparatopic Molecules have Low Polyreactivity

Polyreactive binding can be correlated with poor PK outcomes in humans. Briefly, plates were coated with 1 μg/mL dsDNA in 1×PBS at 4° C. overnight. Plates were blocked with 1×PBS with 1% BSA. Antibodies were tested in triplicate for binding at 100 nM. Antibody binding was detected by Anti-Kappa-HRP antibody, signal is background subtracted for coated wells with secondary antibody only. Test articles included PD1AB53, PD1AB37, PD1AB38, a negative, and a positive control, each at 100 nM, 1-nM, or 1 nM. PD1AB53, PD1AB37, and PD1AB38 showed low polyreactivity at all concentrations, and PD1AB53 showed the lowest polyreactivity out of all test articles. PD1AB37 showed lower polyreactivity than PD1AB38. Biparatopic molecules showed low polyreactivity.

### Example 11. Biparatopic Molecules Show No Self-Interaction

Antibody self-interaction may be indicative of potential risk of poor PK or high viscosity in formulation. Briefly, gold nanoparticles coated with anti-human Ig capture antibody were incubated with 100 nM test article, control antibody, or buffer for 2 hours. Absorbance was measured from 510-570 nm to determine wavelength of maximum absorbance for each antibody (plasmon wavelength). ΔPeak Absorbance wavelength is calculated in comparison with buffer control. Bispecific molecules showed very little self-interaction by AC-SINS, which was shown by a shift in the wavelength of maximum absorbance. Accordingly, biparatopic molecules exhibit low self-interaction.

### Example 12. Biparatopic Molecules Show Favorable Thermal Stability

Test article fluorescence was measured from 25-95° C. with a 1° C./min increase. Tm was calculated from local maxima of the 1st order differential equation and Tagg were measured by absorbance at 266 and 473 nm. Generally, Tagg266 indicates temperature of initial small aggregate formation while Tagg473 indicates temperature of initial large aggregate formation. PD1AB53 showed Tm1 of 68° C., with Tm2 and 3 both slightly over 80° C. PD1AB37 and PD1AB38 showed similar measurements for Tm1 of 67° C. and Tm2 of 73, and Tm3 of 75° C. PD1AB53 showed Tagg266 of just below 80° C. and Tagg473 of slightly over 80° C. PD1AB37 showed Tagg266 of slightly below 70° C. and Tagg473 of slightly above 70° C. PD1AB38 showed Tagg266 of slightly below 70° C. and Tagg473 of slightly above 70° C. Accordingly, biparatopic molecules show thermal stability.

### Example 13. Biparatopic Molecules Show Low Charge Heterogeneity

The sample was diluted in a matrix of methyl cellulose, 4 M urea, 3-10 Pharmalyte® ampholytes (4%), 5 mM Arginine, and pI markers (indicated below). The mixture was submitted to an iCE3 IEF Analyzer (ProteinSimple) and pre-focused at 1,500 V followed by focusing at 3,000 V. The isoelectric points of each peak were calculated from the bracketing pI markers. PD1AB53 and PD1AB38 showed pI values greater than 8.0. PD1AB53 and PD1AB38 have pI profiles that are favorable for formulation and purification process development.

### Example 14. PD1AB53 has Correct Mass by Mass Spectrometry

Sample was denatured and/or reduced by guanidine and DTT and then deglycosylated by PNGase F (MEDNA Bio M3104). The sample was analyzed by Waters ACQUITY UPLC coupled to Xevo G2-XS QTOF mass spectrometer using an ACQUITY UPLC Protein BEH SEC column. Glycosylated conditions assume glycosylation weight of GOF (1445 Da). Nonreduced condition assumes disulfide-bond weight of −2 Da per disulfide (22). Empirically determined molecule mass of PD1AB53 in non-reduced glycosylated state was 201295 Da, and expected molecular mass was 201296 Da. Empirically determined molecule mass of PD1AB53 in non-reduced deglycosylated state was 198406 Da, and expected molecular mass was 198412 Da. Empirically determined molecule mass of PD1AB53 in reduced glycosylated state was 76416 Da and 24255 Da, and expected molecular mass was 76415 Da and 24258 Da, respectively. Empirically determined molecule mass of PD1AB53 in reduced deglycosylated state was 74970 Da and 24253 Da, and expected molecular mass was 74970 Da and 24258 Da, respectively. Empirically determined masses aligned with the calculated mass for each condition within 6 Da.

### Example 15. PD1AB37 has Correct Mass by Mass Spectrometry

Sample was denatured and/or reduced by guanidine and DTT and then deglycosylated by PNGase F (MEDNA Bio M3104). The sample was analyzed by Waters ACQUITY UPLC coupled to Xevo G2-XS QTOF mass spectrometer using an ACQUITY UPLC Protein BEH SEC column. Glycosylated conditions assume glycosylation weight of GOF (1445 Da). Nonreduced condition assumes disulfide-bond weight of −2 Da per disulfide (22). Empirically determined molecule mass of PD1AB37 in non-reduced glycosylated state was 200545 Da, and expected molecular mass was 200545 Da. Empirically determined molecule mass of PD1AB37 in non-reduced deglycosylated state was 197655 Da, and expected molecular mass was 197658 Da. Empirically determined molecule mass of PD1AB37 in reduced glycosylated state was 77122 Da and 23174 Da, and expected molecular mass was 77121 Da and 23175 Da, respectively. Empirically determined molecule mass of PD1AB37 in reduced deglycosylated state was 75676 Da and 23174 Da, and expected molecular mass was 65675 Da and 23175 Da, respectively. Empirically determined masses aligned with the calculated mass for each condition within 2 Da.

### Example 16. PD1AB38 has Correct Mass by Mass Spectrometry

Sample was denatured and/or reduced by guanidine and DTT and then deglycosylated by PNGase F (MEDNA Bio M3104). The sample was analyzed by Waters ACQUITY UPLC coupled to Xevo G2-XS QTOF mass spectrometer using an ACQUITY UPLC Protein BEH SEC column. Glycosylated conditions assume glycosylation weight of GOF (1445 Da). Nonreduced condition assumes disulfide-bond weight of −2 Da per disulfide (22). Empirically determined molecule mass of PD1AB38 in non-reduced glycosylated state was 200765 Da, and expected molecular mass was 200767 Da. Empirically determined molecule mass of PD1AB38 in non-reduced deglycosylated state was 197877 Da, and expected molecular mass was 197880 Da. Empirically determined molecule mass of PD1AB38 in reduced glycosylated state was 77134 Da and 23266 Da, and expected molecular mass was 77134 Da and 23271 Da, respectively. Empirically determined molecule mass of PD1AB38 in reduced deglycosylated state was 75690 Da and 23264 Da, and expected molecular mass was 75689 Da and 23271 Da, respectively. Empirically determined masses aligned with the calculated mass for each condition within 7 Da.

### Example 17. PD1AB38, PD1AB37, and PD1AB53 Exhibit Minimal Post-Translational Modification in the Variable Domain CDRs

Sample was treated with DTT and IAM, followed by trypsin/Lys-C digestion. The digested sample was then analyzed by Waters ACQUITY UPLC coupled to Xevo G2-XS QTOF mass spectrometer using a Protein BEH C18 column. Data showed minimal modifications in the variable domains CDRs of PD1AB38, PD1AB37, and PD1AB53, but no major oxidation or deamination. PD1AB38, PD1AB37, and PD1AB53 show favorable properties for manufacturing.

### Example 18. PD1AB53, PD1AB37, and PD1AB38 Show Similar Glycosylation Patterns

Sample was processed with Waters GlycoWorks RapiFluor-MS N-Glycan Kit. The sample was analyzed by Waters ACQUITY UPLC coupled to Xevo G2-XS QTOF mass spectrometer using an ACQUITY UPLC Glycan BEH Amide Column. Glycan species were calculated as % total. PD1AB53 comprised 0.98% of G0-GN; 8.6% GOF-GN; 0.67% G0; 77.37% GOF; 7.9% ManS; 1.78% GOF+GN; 2.31% G1F; and 0.39% Man6. PD1AB37 comprised 1.11% G0-GN; 9.57% GOF-GN; 0.68% G0; 77.02% GOF; 7.96% ManS; 2.02% GOF+GN; 0.99% G1F; 0.29% Man6; and 0.37% unknown (1368.6). PD1AB38 comprised 1.28% G0-GN; 10.5% GOF-GN; 0.76% G0; 73.29% GOF; 9.44% ManS; 2.26% GOF+GN; 1.31% G1F; 0.28% Man6; 0.29% unknown (1725.73); and 0.59% unknown (1368.6). Accordingly, the predominant species, across tested biparatopic molecules, included GOF, GOF-GN, and Mannose 5.

### Example 19. Epitope Mapping Suggests Unique Binding to PD-1

Epitope mapping was conducted using a peptide cross-linking mass spectrometry approach followed by computational molecular modeling to verify % overlap of various epitopes, and according to standard protocols. Results showed the following % overlap with other molecules:

| PDMoiety 1_XLMS | | | | | | |
|---|---|---|---|---|---|---|
| CC-90006Fab | 0 | | | | | |
| Nivolumab | 0 | 5 | | | | |
| Pembrolizumab | 5 | 0 | 12.9 | | | |
| PDMoiety 2 | 3.2 | 8.3 | 0 | 0 | | |
| hPD-L1 | 7.7 | 0 | 10.5 | 38.5 | 0 | |
| | PDMoiety 1_XLMS | CC- | Nivolumab | Pembrolizumab | PDMoiety2 | hPD- |
| | | 90006Fab | | | | L1 |

Accordingly, epitope mapping revealed diversity of binding sited amongst PD-1 agonists and antagonists.

### Example 20. Biparatopic Molecules do not have PD-1 Antagonist Activity

PD-1 reporter Jurkat cells were incubated with the indicated concentrations of test articles for 1 hour. PD-L1 expressing cells were then added and SHP-2 recruitment was assessed after 2 hours. PD1AB43, PD1AB38, PD1AB37, and PD1AB53, LY3462817 negative control, and TTJ2 negative control showed no antagonist activity, as compared to Pembrolizumab positive control. Accordingly, biparatopic molecules PD1AB43, PD1AB38, PD1AB37, and PD1AB53 do not exhibit antagonist activity.

### Example 21. Biparatopic Molecules have PD-1 Agonist Activity

PD-1 reporter Jurkat cells were incubated with concentrations of soluble test articles ranging from 0.001 to 100 nM. SHP-2 recruitment was assessed after 2 hours. PD1AB43 showed EC50(nM) of 0.5736; PD1AB38 showed EC50 of 0.3022; PD1AB37 showed EC50 of 0.3700; and PD1AB53 showed EC50 of 1.210.

In another experiment, plates were coated with anti-human IgG, blocked, and Ig-tethered test articles were added for 1 hour at concentrations ranging from 0.001 to 100 nM. Plates were washed and PD-1 reporter Jurkat cells were added. SHP-2 recruitment was assessed after 2 hours. PD1AB43 showed EC50(nM) of 8.507; PD1AB38 showed EC50 of 20.85; PD1AB37 showed EC50 of 24.48; PD1AB53 showed EC50 of 4.835; and LY3462817 showed EC50 of 5.686. Accordingly, soluble and Ig-tethered biparatopic molecules show agonist activity.

### Example 22. Biparatopic Molecules Inhibit T Cell Proliferation In Vitro

Human PBMCs were stimulated with Staphylococcus enterotoxin B (SEB) or a peptide pool containing epitopes from CMV, EBV, and influenza in the absence or presence of 100 nM control test article, LY3462817, PD1AB38, PD1AB37, or PD1AB53. Proliferation was assessed by intracellular staining for Ki67 at 3 to 6 days after stimulation. Percent Ki67 positive events were normalized relative to the no test article condition. Data showed inhibition of SEB- or CEF peptide pool-stimulated T cell proliferation. Accordingly, biparatopic molecules inhibit proliferation of human T cells.

### Example 23. Biparatopic Molecules Inhibit T Cell Proliferation In Vitro

Human PBMC were pretreated for 2 h at 37° C. with vehicle or 200 nM PD1AB53, and then stimulated with tetanus toxoid (5 μg/ml) in the presence of antibodies blocking PD-L1 and PD-L2 (2 ng/ml each). Levels of interferon gamma (IFN-γ), IL-2, and tumor necrosis factor (TNF-α) were measured by MSD ELISA after 4 days of stimulation and used to calculate % inhibition. Data showed decrease of IFN-γ, IL-2, and TNF-α. PD1AB53 decreased tetanus toxoid-induced cytokine production in PBMCs.

### Example 24. Biparatopic Molecules Inhibit CpGA-Induced Type I Interferon Response in Plasmacytoid Dendritic Cells In Vitro

Without wishing to be bound to a particular theory, TLR9 activation can induce PD-1 expression on plasmacytoid dendritic cells. Purified human plasmacytoid dendritic cells were stimulated with CpGA for 6 or 24 hours, followed by cell collection and RNA extraction for qRT-PCR measurement of PD-1, IFN-β1, MX1, MX2, and IFIT3, as well as supernatant collection for cytokine analysis by MSD. Gene expression data showed expression of PD-1, IFN-β, MX1, MX2, and IFIT3 at 6 hours only following stimulation, as compared to unstimulated cells. Analysis of supernatants showed secretion of IFN-β1, TNFα, and IL-13 at 6 and 24 hours following stimulation as compared to supernatants from unstimulated cells.

Next, purified human plasmacytoid dendritic cells were stimulated with CpGA for 24 hours in the presence or absence of PD1AB38, PD1AB37, or PD1AB53. CpGA stimulation induced PD-1 expression on the cell surface and induced Type I IFN response genes OAS1, IFIT3, MX1 and IFN-β1. CpGA-induced expression of these genes was inhibited in the presence of PD1AB38, PD1AB37, and PD1AB53. Accordingly, biparatopic PD-1 agonists suppress CpGA-induced Type I IFN response genes.

### Example 25. PD1AB43 Prolongs Survival and Ameliorates Skin Phenotype in xGVHD

Xenogeneic graft versus host disease was induced by the transfer of human PBMC into immunodeficient mice. Beginning 10 days after cell transfer, mice were treated subcutaneously weekly with vehicle or PD1AB43. Skin inflammation was scored as follows: 0: healthy, 1: hair loss<1 cm×1 cm, 2: hair loss>1 cm×1 cm, but not total, 3: total hair loss, plus an additional 0.3 each for inflamed tail, ear, foot. PD1AB43 improved skin phenotype, and prolonged median survival time to 70.5 days as compared to 53 days for the vehicle.

### Example 26. PD1AB53 Decreases Severity of Skin Inflammation in xGVHD

Xenogeneic graft versus host disease was induced by the transfer of human PBMC into immunodeficient mice. Beginning 10 days after cell transfer, mice were treated subcutaneously every 2 weeks with vehicle or PD1AB53. Skin inflammation was scored as follows: 0: healthy, 1: hair loss<1 cm×1 cm, 2: hair loss>1 cm×1 cm, but not total, 3: total hair loss, plus an additional 0.3 each for inflamed tail, ear, foot. PD1AB53 showed improved skin score as compared to vehicle.

### Example 27. PD1AB43 Decreases T Cell Infiltration of the Skin and Colon in xGVHD

Xenogeneic graft versus host disease was induced by the transfer of human PBMC into immunodeficient mice. Beginning 10 days after cell transfer, mice were treated subcutaneously weekly with vehicle or PD1AB43. Skin or colon was harvested>100 days post-engraftment. Samples were either formalin fixed and paraffin embedded or digested to isolate infiltrating cells. Infiltrating CD4+ and CD8+ T cells were quantified by automated image analysis of IHC samples or flow cytometry of digested tissues. Skin histopathology was scored by a pathologist blinded to the groups. PD1AB43 treated mice exhibited lower levels of CD4+ and CD8+ cells in skin and colon, as measured by IHC or flow cytometry. PD1AB43 treated mice also showed lower levels of CD4+ cells in blood, and decreased histopathology score, indicative of improved skin phenotype. PD1AB43 effectively decreases T cell infiltration of the skin and colon, and improves skin phenotype in xGVHD.

### Example 28. Biparatopic Molecules Increase Regulatory T Cell Co-Expressing Activation Markers in Human PD-1 Knock-In Mice

Eight week old human PD-1 knock-in mice were dosed subcutaneously in the dorsal scruff with PD1AB43, PD1AB38, PD1AB37, PD1AB53, PD1AB64, LY3462817, or vehicle. At days 4, 7, or 10 mice were euthanized, spleens dissected and splenocytes stained with antibodies against surface and intracellular markers to immunophenotype T cell subsets and measure expression of the activation markers LAG3 and CTLA4. Data showed increase in the frequency and number of splenic Treg co-expressing LAG3 and CTLA4 at days 4, 7, and 10 following injection. PD1AB43, PD1AB38, PD1AB37, PD1AB53, and PD1AB64 biparatopic molecules increase the expression of regulatory T cell activation markers in human PD-1 knock-in mice.

### Example 29. Biparatopic Molecules Increase Regulatory T Cell Co-Expressing Activation Markers in a Dose-Dependent Manner

Eight week old human PD-1 knock-in mice were dosed subcutaneously in the dorsal scruff with low, medium, and high dose of PD1AB53, PD1AB37, PD1AB38, D1.3, LY3462817, or vehicle. After 7 days the mice were euthanized, spleens dissected and splenocytes stained with antibodies against surface and intracellular markers to immunophenotype T cell subsets and measure expression of the activation markers LAG3 and CTLA4. Data show dose-dependent increase in the frequency and number of splenic Treg co-expressing LAG3 and CTLA4. PD1AB38, PD1AB37, and PD1AB53 biparatopic molecules increase the expression of regulatory T cell activation markers in a dose-dependent manner.

### Example 30. Biparatopic Molecules Increase Regulatory T Cell Co-Expressing Activation Markers in Human CD34+ Engrafted NSG Mice

Human CD34-positive hematopoietic stem cell-engrafted NSG mice were dosed subcutaneously in the dorsal scruff with PD1AB38, PD1AB37, PD1AB53, D1.3, LY3462817, or vehicle. After 7 days the mice were euthanized, spleens dissected and splenocytes stained with antibodies against surface and intracellular markers to immunophenotype human T cell subsets and measure expression of the activation markers LAG3 and CTLA4. Data showed increase in the frequency and number of splenic human Treg co-expressing LAG3 and CTLA4. PD1AB38, PD1AB37, and PD1AB53 biparatopic molecules increase the expression of regulatory T cell activation markers in human CD34+ engrafted NSG mice.

These data demonstrate that the PD-1 antibodies can act as agonists and function with greater effectiveness in a biparatopic format, such as those illustrated in FIG. 2, as compared to a monoparatopic format where each binding domain binds to the same epitope.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1-50. (canceled)

51. A protein comprising a first binding domain, a second binding domain, a third binding domain, and a fourth binding domain, wherein each said binding domain binds to PD-1, wherein at least one of the binding domains comprises a first heavy chain variable region comprising a first CDR of SEQ ID NO: 267, a second CDR of SEQ ID NO: 229, and a third CDR of SEQ ID NO: 230; and a first light chain variable region comprising a first CDR of SEQ ID NO: 174, a second CDR of SEQ ID NO: 227, and a third CDR of SEQ ID NO: 176.

52. The protein of claim 51, wherein said first heavy chain variable region comprises the sequence of SEQ ID NO: 257, and wherein said first light chain variable region comprises the sequence of SEQ ID NO: 170.

53. The protein of claim 51, wherein only two of the binding domains comprise said first heavy chain variable region and said first light chain variable region.

54. The protein of claim 51, wherein the protein is a PD-1 agonist.

55. The protein of claim 51, wherein the first binding domain and the second binding domain are in a Fab format.

56. The protein of claim 55, wherein the third binding domain and the fourth binding domain are in an scFv format.

57. The protein of claim 51, wherein at least one of the other binding domains comprises
a second heavy chain variable region comprising a first CDR of SEQ ID NO: 238, a second CDR of SEQ ID NO: 239, and a third CDR of SEQ ID NO: 240; and a second light chain variable region comprising a first CDR of SEQ ID NO: 40, a second CDR of SEQ ID NO: 237, and a third CDR of SEQ ID NO: 41;
a second heavy chain variable region comprising a first CDR of SEQ ID NO: 238, a second CDR of SEQ ID NO: 264, and a third CDR of SEQ ID NO: 240; and a second light chain variable region comprising a first CDR of SEQ ID NO: 40, a second CDR of SEQ ID NO: 237, and a third CDR of SEQ ID NO: 41; or
a second heavy chain variable region comprising a first CDR having the residues SYYMH, a second CDR of SEQ ID NO: 239, and a third CDR of SEQ ID NO: 240; and a second light chain variable region comprising a first CDR of SEQ ID NO: 276, a second CDR of SEQ ID NO: 237, and a third CDR of SEQ ID NO: 41.

58. The protein of claim 57, wherein
said second heavy chain variable region comprises the sequence of SEQ ID NO: 262, and wherein said second light chain variable region comprises the sequence of SEQ ID NO: 261;
said second heavy chain variable region comprises the sequence of SEQ ID NO: 202, and wherein said second light chain variable region comprises the sequence of SEQ ID NO: 36;
said second heavy chain variable region comprises the sequence of SEQ ID NO: 275, and wherein said second light chain variable region comprises the sequence of SEQ ID NO: 36; or
said second heavy chain variable region comprises the sequence of SEQ ID NO: 256, and wherein said second light chain variable region comprises the sequence of SEQ ID NO: 259.

59. The protein of claim 57, wherein two of the binding domains comprise said second heavy chain variable region and said second light chain variable region.

60. The protein of claim 51, comprising a first polypeptide chain and a second polypeptide chain, wherein:
the first polypeptide chain has a formula of from N-terminus to C-terminus:
[VH-A]-[CH1]-[CH2]-[CH3]-[Linker 1]-[VH-B]-[Linker 2]-[VK-B]; or
[VH-A]-[CH1]-[CH2]-[CH3]-[Linker 1]-[VK-B]-[Linker 2]-[VH-B]; and
the second polypeptide chain has a formula of from N-terminus to C-terminus:
[VK-A]-[CK]; or
the first polypeptide chain has a formula of from N-terminus to C-terminus:
[VH-B]-[CH1]-[CH2]-[CH3]-[Linker 1]-[VH-A]-[Linker 2]-[VK-A]; or
[VH-B]-[CH1]-[CH2]-[CH3]-[Linker 1]-[VK-A]-[Linker 2]-[VH-A]; and
the second polypeptide chain has a formula of from N-terminus to C-terminus:
[VK-B]-[CK],
wherein:
VH-A is the first heavy chain variable region;
VK-A is the first light chain variable region;
VH-B is the second heavy chain variable region;
VK-B is the second light chain variable region;
CH1 is a constant heavy domain 1 of human IgG1;
CH2 is a constant heavy domain 2 of human IgG1;
CH3 is a constant heavy domain 3 of human IgG1;
CK is a constant domain of kappa light chain;
Linker 1 is a glycine/serine, glycine/alanine, glycine/glutamic acid/serine, or alanine/glutamic acid/lysine linker; and
Linker 2 is a glycine/serine, glycine/alanine, glycine/glutamic acid/serine, or alanine/glutamic acid/lysine linker.

61. The protein of claim 60, wherein Linker 1 comprises the sequence of (GGGGS)n (SEQ ID NO: 303), wherein n is 1-4; and wherein Linker 2 comprises the sequence of (GGGGS)n (SEQ ID NO: 303), (GGGSE)n (SEQ ID NO: 305), or (GGGGA)n (SEQ ID NO: 304), wherein each n is independently, 1-4.

62. The protein of claim 60, wherein
the first polypeptide chain comprises from N-terminus to C-terminus the sequences of SEQ ID NO: 256, 12, 6, 257, 7, and 170; and the second polypeptide chain comprises from N-terminus to C-terminus the sequences of SEQ ID NO: 259 and 274;
the first polypeptide chain comprises from N-terminus to C-terminus the sequences of SEQ ID NO: 275, 12, 6, 257, 7, and 170; and the second polypeptide chain comprises from N-terminus to C-terminus the sequences of SEQ ID NO: 36 and 274;
the first polypeptide chain comprises from N-terminus to C-terminus the sequences of SEQ ID NO: 202, 12, 6, 257, 7, and 170; and the second polypeptide chain comprises from N-terminus to C-terminus the sequences of SEQ ID NO: 36 and 274; or
the first polypeptide chain comprises from N-terminus to C-terminus the sequences of SEQ ID NO: 257, 12, 1, 261, 7, and 262; and the second polypeptide chain comprises from N-terminus to C-terminus the sequences of SEQ ID NO: 170 and 274.

63. A pharmaceutical composition comprising the protein of claim 51 and a pharmaceutically acceptable carrier.

64. A method of treating an unwanted immune response in a subject, comprising administering to the subject an effective amount of the pharmaceutical composition of claim 63.

65. The method of claim 64, wherein the unwanted immune response comprises Systemic Lupus Erythematosus (SLE); Aicardi-Goutieres syndrome; bilateral striatal necrosis; chronic atypical neutrophilic dermatosis with lipodystrophy and elevated temperature (CANDLE); complete non-penetrance; dyschromatosis symmetrica hereditaria; familial chilblain lupus; Japanese autoinflammatory syndrome with lipodystrophy (JASL); joint contractures, muscle atrophy, microcytic anaemia, panniculitis, and lipodystrophy (JMP); Mendelian susceptibility to mycobacterial disease (MSMD); Nakajo-Nishimura syndrome; retinal vasculopathy with cerebral leukodystrophy (RVCL); spastic paraparesis; STING-associated vasculopathy with onset in infancy (SAVI); Singleton-Merten syndrome; spondylochondromatosis (SPENCD); an autoimmune disorder; inflammatory bowel disease; autoimmune hepatitis; primary sclerosing cholangitis; Type 1 diabetes; a transplant; GVHD; Crohn's disease; or ulcerative colitis.

66. A protein comprising a first polypeptide chain and a second polypeptide chain, wherein the first polypeptide chain comprises from N-terminus to C-terminus the sequences of SEQ ID NO: 257, 12, 1, 261, 7, and 262; and the second polypeptide chain comprises from N-terminus to C-terminus the sequences of SEQ ID NO: 170 and 274.

67. A pharmaceutical composition comprising the protein of claim 66 and a pharmaceutically acceptable carrier.

68. A method of treating an unwanted immune response in a subject, comprising administering to the subject an effective amount of the pharmaceutical composition of claim 67.

69. The method of claim 68, wherein the unwanted immune response comprises Systemic Lupus Erythematosus (SLE); Aicardi-Goutieres syndrome; bilateral striatal necrosis; chronic atypical neutrophilic dermatosis with lipodystrophy and elevated temperature (CANDLE); complete non-penetrance; dyschromatosis symmetrica hereditaria; familial chilblain lupus; Japanese autoinflammatory syndrome with lipodystrophy (JASL); joint contractures, muscle atrophy, microcytic anaemia, panniculitis, and lipodystrophy (JMP); Mendelian susceptibility to mycobacterial disease (MSMD); Nakajo-Nishimura syndrome; retinal vasculopathy with cerebral leukodystrophy (RVCL); spastic paraparesis; STING-associated vasculopathy with onset in infancy (SAVI); Singleton-Merten syndrome; spondylochondromatosis (SPENCD); an autoimmune disorder; inflammatory bowel disease; autoimmune hepatitis; primary sclerosing cholangitis; Type 1 diabetes; a transplant; GVHD; Crohn's disease; or ulcerative colitis.
